(19) United States
(12) Patent Application Publication
(10) Pub. No.: US 2016/0176880 A1
(43) Pub. Date: Jun. 23, 2016

(54) SUBSTITUTED IMIDAZO[1,2-A]PYRAZINECARBOXAMIDES AND USE THEREOF

(71) Applicant: BAYER PHARMA AKTIENGESELLSCHAFT, Berlin, OT (DE)

(72) Inventors: Alexandros VAKALOPOULOS, Hilden (DE); Markus FOLLMANN, Köln (DE); Ingo HARTUNG, Berlin (DE); Philipp BUCHGRABER, Berlin (DE); Rolf JAUTELAT, Haan (DE); Niels LINDNER, Wuppertal (DE); Frank WUNDER, Wuppertal (DE); Johannes-Peter STASCH, München (DE); Gorden REDLICH, Bochum (DE); Lisa Dietz, Wuppertal (DE); Volkhart Min-Jian LI, Velbert (DE)

(73) Assignee: BAYER PHARMA AKTIENGESELLSCHAFT, Berlin, OT (DE)

(21) Appl. No.: 14/910,519

(22) PCT Filed: Aug. 5, 2014
(86) PCT No.: PCT/EP2014/066758

§ 371 Date: Feb. 5, 2016

(30) Foreign Application Priority Data
Aug. 8, 2013 (EP) 13179783.9
May 2, 2014 (EP) 14166913.5

(51) Int. Cl. C07D 487/04; A61K 31/5377; A61K 31/4985; C07D 519/00; A61K 45/06

(52) U.S. Cl. CPC C07D 487/04; C07D 519/00; A61K 45/06; A61K 31/4985; A61K 31/5377

(57) ABSTRACT

The present application relates to novel substituted imidazo[1,2-a]pyrazinecarboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

The present application relates to novel substituted imidazo[1,2-a]pyrazinecarboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory centre. This is of central importance for the activation mechanism. NO is able to bind to the iron atom of haem and thus markedly increase the activity of the enzyme. Haem-free preparations cannot, by contrast, be stimulated by NO. Carbon monoxide (CO) is also able to bind to the central iron atom of haem, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attack at the central iron atom of haem. In addition to the side effects, the development of tolerance is one of the crucial disadvantages of this mode of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

WO 89/03833 A1 and WO 96/34866 A1, among other documents, disclose various imidazo[1,2-a]pyrazine derivatives which can be used for treatment of disorders.

It was an object of the present invention to provide novel substances which act as stimulators of soluble guanylate cyclase and are suitable as such for treatment and/or prophylaxis of diseases.

The present invention provides compounds of the general formula (I)

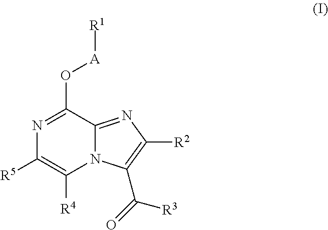

in which
A is CH₂, CD₂ or CH(CH₃),
R¹ is (C₄-C₆)-alkyl, (C₃-C₇)-cycloalkyl, pyridyl or phenyl,
   where (C₄-C₆)-alkyl may be up to hexasubstituted by fluorine,
   where (C₃-C₇)-cycloalkyl may be substituted by 1 to 4 substituents selected independently from the group of fluorine, trifluoromethyl and (C₁-C₄)-alkyl,
   where pyridyl is substituted by 1 or 2 substituents selected independently from the group of halogen, cyano and (C₁-C₄)-alkyl,
   and
   where phenyl may be substituted by 1 to 4 substituents selected independently from the group of halogen, cyano, monofluoromethyl, difluoromethyl, trifluoromethyl, (C₁-C₄)-alkyl, (C₂-C₃)-alkynyl, (C₁-C₄)-alkoxy, (C₃-C₅)-cycloalkyl, difluoromethoxy and trifluoromethoxy, or may be substituted on two adjacent carbon atoms in the phenyl by a difluoromethylenedioxy bridge,
R² is hydrogen, (C₁-C₄)-alkyl, (C₁-C₄)-alkoxymethyl, cyclopropyl, monofluoromethyl, difluoromethyl or trifluoromethyl,
R³ is a group of the formula

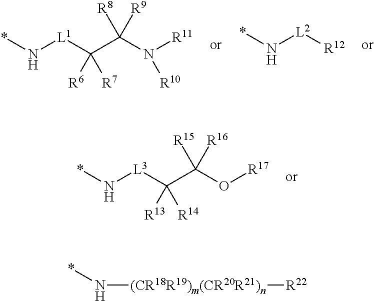

where
* is the attachment site to the carbonyl group,
L¹ is a bond, methanediyl or 1,2-ethanediyl,
   in which methanediyl and 1,2-ethanediyl may be substituted by 1 or 2 substituents selected independently from the group of fluorine, trifluoromethyl, (C₁-C₄)-alkyl, (C₃-C₅)-cycloalkyl, hydroxyl and (C₁-C₄)-alkoxy,
L² is a bond or (C₁-C₄)-alkanediyl,
   in which (C₁-C₄)-alkanediyl may be substituted by 1 to 3 substituents selected independently from the group of fluorine, trifluoromethyl, (C₁-C₄)-alkyl, (C₃-C₅)-cycloalkyl, hydroxyl and (C₁-C₄)-alkoxy,
L³ is a bond, methanediyl or 1,2-ethanediyl,
   in which methanediyl or 1,2-ethanediyl may be substituted by 1 or 2 substituents selected independently from the group of fluorine, trifluoromethyl, (C₁-C₄)-alkyl, (C₃-C₇)-cycloalkyl, hydroxyl and (C₁-C₄)-alkoxy,
R⁶ is hydrogen, (C₁-C₆)-alkyl, (C₂-C₆)-alkenyl, (C₂-C₆)-alkynyl, (C₃-C₇)-cycloalkyl, 5- or 6-membered heteroaryl or phenyl,
   in which (C₁-C₆)-alkyl may be substituted by 1 to 3 substituents selected independently from the group of fluorine, trifluoromethyl, difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy,
      in which phenyl, phenoxy and benzyloxy may be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   and
   in which phenyl and 5- or 6-membered heteroaryl may be substituted by 1 to 3 substituents selected independently from the group of halogen, cyano, nitro, trifluoromethyl, (C₁-C₄)-alkyl, (C₁-C₄)-alkoxy, difluoromethoxy, trifluoromethoxy and (C₁-C₄)-alkylsulphonyl,
R⁷ is hydrogen or (C₁-C₆)-alkyl,
or
R⁶ and R⁷ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
R⁸ is hydrogen, (C₁-C₆)-alkyl, (C₃-C₇)-cycloalkyl, (C₂-C₆)-alkenyl, (C₂-C₆)-alkynyl, 5- or 6-membered heteroaryl or phenyl,
   in which (C₁-C₆)-alkyl may be substituted by 1 to 3 substituents selected independently from the group of fluorine, difluoromethyl, trifluoromethyl, difluoromethoxy, trifluoromethoxy, hydroxyl, (C₁-C₄)-alkoxy, benzyloxy, phenoxy and phenyl,
      in which benzyloxy, phenoxy and phenyl may be substituted by 1 to 3 halogen substituents,
   in which (C₃-C₇)-cycloalkyl may be substituted by 1 or 2 fluorine or (C₁-C₄)-alkyl substituents,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R⁹ is hydrogen or (C₁-C₆)-alkyl,
or
R⁸ and R⁹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
or
R⁶ and R⁸ together with the carbon atoms to which they are bonded form a 3- to 7-membered carbocycle or a 4- to 7-membered heterocycle,
with the proviso that not more than one of the R⁶ and R⁷, R⁸ and R⁹, and R⁶ and R⁸ radical pairs at the same time forms a carbo- or heterocycle,
with the proviso that the R⁶ and R⁸ radicals are not both simultaneously phenyl or 5- or 6-membered heteroaryl,
R¹⁰ is hydrogen or (C₁-C₄)-alkyl,
   in which (C₁-C₄)-alkyl may be substituted by 1 to 3 substituents selected independently from the group of fluorine, trifluoromethyl, hydroxyl and (C₁-C₄)-alkoxy,
R¹¹ is hydrogen, (C₁-C₆)-alkyl, (C₃-C₇)-cycloalkyl, phenyl or benzyl,
   in which (C₁-C₆)-alkyl may be substituted by 1 to 3 substituents selected independently from the group of fluorine, trifluoromethyl, hydroxyl, (C₁-C₄)-alkoxy and phenoxy,
   and
   in which phenyl and benzyl may be substituted by 1 to 3 substituents selected independently from the group of halogen and trifluoromethyl,
or
R¹⁰ and R¹¹ together with the nitrogen atom to which they are bonded form a 4- to 7-membered azaheterocycle,
R¹² is 5- to 9-membered azaheterocyclyl bonded via a ring carbon atom,
   in which 5- to 9-membered azaheterocyclyl may be substituted by 1 to 5 substituents selected independently from the group of fluorine, trifluoromethyl, (C₁-C₄)-alkyl, (C₃-C₇)-cycloalkyl and benzyl,
   and
   in which 5- to 9-membered azaheterocyclyl may be fused to a phenyl ring which may in turn be substituted by 1 or 2 substituents selected from halogen, (C₁-C₄)-alkyl and trifluoromethyl,
R¹³ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, —(C═O)NR²³R²⁴, 5- or 6-membered heteroaryl or phenyl,
   in which (C₁-C₆)-alkyl may be substituted by 1 to 3 substituents selected independently from the group of fluorine, trifluoromethyl, difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy,
      in which phenyl, phenoxy and benzyloxy may in turn be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   in which R²³ is hydrogen, (C₁-C₄)-alkyl, (C₃-C₇)-cycloalkyl, aryl or naphthyl,
   in which R²⁴ is hydrogen or methyl,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁴ is hydrogen or (C₁-C₆)-alkyl,
   in which (C₁-C₄)-alkyl may be substituted by hydroxyl,
or
R¹³ and R¹⁴ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
R¹⁵ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, 5- or 6-membered heteroaryl or phenyl,
   in which (C₁-C₆)-alkyl may be substituted by 1 to 3 substituents selected independently from the group of fluorine, trifluoromethyl, difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy,
      in which phenyl, phenoxy and benzyloxy may in turn be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁶ is hydrogen or (C₁-C₆)-alkyl,
   in which (C₁-C₄)-alkyl may be substituted by hydroxyl,
or
R¹⁵ and R¹⁶ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
with the proviso that the R¹³ and R¹⁵ radicals are not both simultaneously phenyl or 5- or 6-membered heteroaryl,
or
R¹³ and R¹⁵ together with the carbon atoms to which they are bonded form a 3- to 7-membered carbocycle or a 4- to 7-membered heterocycle,
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
   in which (C₁-C₄)-alkyl may be substituted by 1 to 5 fluorine substituents,
m is 0, 1 or 2,
n is 0 or 1,
R¹⁸ is hydrogen, cyano or (C₁-C₄)-alkyl,
   in which (C₁-C₄)-alkyl may be substituted by 1 to 5 fluorine substituents,
R¹⁹ is hydrogen or (C₁-C₄)-alkyl,
   in which (C₁-C₄)-alkyl may be substituted by 1 to 5 fluorine substituents,
R²⁰ is hydrogen or (C₁-C₄)-alkyl,
   in which (C₁-C₄)-alkyl may be substituted by 1 to 5 fluorine substituents,
R²¹ is hydrogen or (C₁-C₄)-alkyl,
   in which (C₁-C₄)-alkyl may be substituted by 1 to 3 fluorine substituents,
or
R¹⁸ and R¹⁹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
or
R²⁰ and R²¹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl, or
R¹⁸ and R²⁰ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, 5- to 9-membered heterocyclyl bonded via a ring carbon atom, 5- to 9-membered carbocyclyl, phenyl, indanyl or 5- to 10-membered heteroaryl,
   where (C₁-C₆)-alkyl may be substituted by 1 to 3 substituents independently selected from the group of fluorine, trifluoromethyl and cyano,
   where phenyl may be substituted by 1 to 3 substituents selected independently from the group of halogen, cyano, trifluoromethyl, difluoromethyl, (C₁-C₆)-alkyl, (C₁-C₄)-alkylcarbonyl, (C₁-C₄)-alkoxycarbonyl, hydroxycarbonyl, —(C═O)NR²⁵R²⁶, (C₁-C₄)-alkylsulphonyl, (C₃-C₆)-cycloalkylsulphonyl, (C₁-C₄)-alkylthio, (C₁-C₄)-alkoxy, trifluoromethoxy, difluoromethoxy, phenoxy, hydroxyl, 5- to 10-membered heteroaryl and (C₃-C₇)-cycloalkyl,
      in which (C₁-C₆)-alkyl may be substituted by 1 or 2 substituents selected independently from the group consisting of fluorine, trifluoromethoxy, (C₁-C₄)-alkylcarbonyl, —(C═O)NR²⁵R²⁶, (C₁-C₄)-alkoxy, (C₃-C₆)-cycloalkyl, morpholinyl, piperidinyl, pyrrolidinyl, piperazinyl, phenyl, hydroxyl and amino,
         in which phenyl may be substituted by 1 to 3 halogen substituents,
         in which amino may be substituted by 1 or 2 substituents selected independently from (C₁-C₆)-alkyl, (C₁-C₄)-alkylcarbonyl, (C₃-C₆)-cycloalkylsulphonyl, (C₁-C₄)-alkylsulphonyl and methoxy-(C₁-C₄)-alkyl,
         in which (C₃-C₆)-cycloalkyl may be substituted by amino or hydroxyl,
         and in which
         R²⁵ and R²⁶ are each independently hydrogen, (C₁-C₄)-alkyl or (C₃-C₇)-cycloalkyl,
   where indanyl may be substituted by 1 or 2 substituents selected independently from the group of fluorine, trifluoromethyl and hydroxyl,
   where 5- to 10-membered heteroaryl may be substituted by 1 to 3 substituents independently of one another selected from the group of fluorine, chlorine, cyano, (C₁-C₆)-alkyl, trifluoromethyl, (C₁-C₄)-alkoxy, amino, (C₁-C₄)-alkoxycarbonyl, hydroxycarbonyl, —(C═O)NR²⁵R²⁶, phenyl, pyridyl, pyrimidyl, 1,3-thiazol-5-yl and (C₃-C₇)-cycloalkyl,
      in which (C₁-C₆)-alkyl may be substituted by 1 to 3 substituents selected independently from the group of halogen, cyano, hydroxyl, amino, trifluoromethyl, difluoromethyl, (C₁-C₄)-alkylsulphonyl, (C₁-C₄)-alkylcarbonyl, (C₁-C₄)-alkoxycarbonyl, hydroxycarbonyl, (C₁-C₄)-alkylthio, (C₁-C₄)-alkoxy, trifluoromethoxy, difluoromethoxy, phenoxy, phenyl, pyridyl, pyrimidyl, 5-membered heteroaryl, tetrahydrothiophenyl 1,1-dioxide, (C₃-C₇)-cycloalkyl, morpholinyl, piperidinyl, pyrrolidinyl, 2-oxopyrrolidin-1-yl, piperazinyl, tetrahydrothiophenyl 1,1-dioxide, thiomorpholinyl 1,1-dioxide and azetidine,
      in which 5-membered heteroaryl may be substituted by 1 to 3 substituents selected independently from the group of halogen, (C₁-C₄)-alkyl and (C₁-C₄)-alkoxy,
      in which piperidinyl may be substituted by 1 to 4 fluorine substituents,
      in which phenyl may be substituted by 1 to 3 substituents selected independently from the group of halogen, (C₁-C₄)-alkyl and (C₁-C₄)-alkoxy,
      in which azetidine may be substituted by hydroxyl,
      in which piperazinyl may be substituted by 1 to 3 substituents selected independently from the group consisting of (C₁-C₄)-alkyl, (C₃-C₇)-cycloalkyl and trifluoromethyl,
      and in which
      R²⁵ and R²⁶ are each independently hydrogen, (C₁-C₄)-alkyl or (C₃-C₇)-cycloalkyl,
   where 5- to 9-membered heterocyclyl bonded via a ring carbon atom may be substituted by 1 or 2 substituents selected independently from the group of oxo, fluorine, hydroxyl and (C₁-C₄)-alkyl,
   and
   where 5- to 9-membered carbocyclyl may be substituted by 1 or 2 substituents selected independently from the group of trifluoromethyl, fluorine, hydroxyl, hydroxycarbonyl, (C₁-C₄)-alkoxycarbonyl and (C₁-C₄)-alkyl,
R⁴ is hydrogen,
R⁵ is hydrogen, halogen, cyano, difluoromethyl, trifluoromethyl, (C₁-C₄)-alkyl, (C₃-C₇)-cycloalkyl, (C₂-C₄)-alkynyl, (C₁-C₄)-alkylamino, difluoromethoxy, trifluoromethoxy, (C₁-C₄)-alkoxy, amino, 4- to 7-membered heterocyclyl or 5- or 6-membered heteroaryl,
   and the N-oxides, salts, solvates, salts of the N-oxides and solvates of the N-oxides or salts thereof.

The present invention provides compounds of the general formula (I)

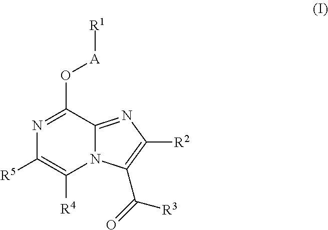

in which
A is CH₂, CD₂ or CH(CH₃),
R¹ is (C₄-C₆)-alkyl, (C₃-C₇)-cycloalkyl, pyridyl or phenyl,
   where (C₄-C₆)-alkyl may be up to hexasubstituted by fluorine,
   where (C₃-C₇)-cycloalkyl may be substituted by 1 to 4 substituents selected independently from the group of fluorine, trifluoromethyl and (C₁-C₄)-alkyl,
   where pyridyl is substituted by 1 or 2 substituents selected independently from the group of halogen, cyano and (C₁-C₄)-alkyl,
   and
   where phenyl may be substituted by 1 to 4 substituents selected independently from the group of halogen, cyano, monofluoromethyl, difluoromethyl, trifluoromethyl, (C₁-C₄)-alkyl, (C₂-C₃)-alkynyl, (C₁-C₄)-alkoxy, (C₃-C₅)-cycloalkyl, difluoromethoxy and trifluoromethoxy, or may be substituted on two adjacent carbon atoms in the phenyl by a difluoromethylenedioxy bridge,
R² is hydrogen, (C₁-C₄)-alkyl, (C₁-C₄)-alkoxymethyl, cyclopropyl, monofluoromethyl, difluoromethyl or trifluoromethyl,
R³ is a group of the formula

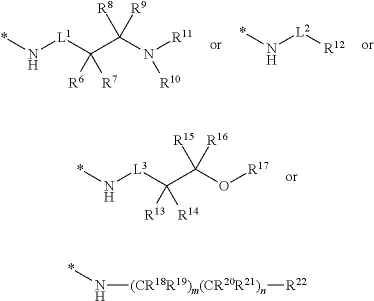

where
* is the attachment site to the carbonyl group,
L¹ is a bond, methanediyl or 1,2-ethanediyl,
   in which methanediyl and 1,2-ethanediyl may be substituted by 1 or 2 substituents selected independently from the group of fluorine, trifluoromethyl, (C₁-C₄)-alkyl, (C₃-C₅)-cycloalkyl, hydroxyl and (C₁-C₄)-alkoxy,
L² is a bond or (C₁-C₄)-alkanediyl,
   in which (C₁-C₄)-alkanediyl may be substituted by 1 to 3 substituents selected independently from the group of fluorine, trifluoromethyl, (C₁-C₄)-alkyl, (C₃-C₅)-cycloalkyl, hydroxyl and (C₁-C₄)-alkoxy,
L³ is a bond, methanediyl or 1,2-ethanediyl,
   in which methanediyl or 1,2-ethanediyl may be substituted by 1 or 2 substituents selected independently from the group of fluorine, trifluoromethyl, (C₁-C₄)-alkyl, (C₃-C₇)-cycloalkyl, hydroxyl and (C₁-C₄)-alkoxy,
R⁶ is hydrogen, (C₁-C₆)-alkyl, (C₂-C₆)-alkenyl, (C₂-C₆)-alkynyl, (C₃-C₇)-cycloalkyl, 5- or 6-membered heteroaryl or phenyl,
   in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
      in which phenyl, phenoxy and benzyloxy may be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   and
   in which phenyl and 5- or 6-membered heteroaryl may be substituted by 1 to 3 substituents selected independently from the group of halogen, cyano, nitro, trifluoromethyl, (C₁-C₄)-alkyl, (C₁-C₄)-alkoxy, difluoromethoxy, trifluoromethoxy and (C₁-C₄)-alkylsulphonyl,
R⁷ is hydrogen or (C₁-C₆)-alkyl,
or
R⁶ and R⁷ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
R⁸ is hydrogen, (C₁-C₆)-alkyl, (C₃-C₇)-cycloalkyl, (C₂-C₆)-alkenyl, (C₂-C₆)-alkynyl, 5- or 6-membered heteroaryl or phenyl,
   in which (C₁-C₆)-alkyl may be substituted by 1 or 2 substituents selected independently from the group of trifluoromethyl, difluoromethoxy, trifluoromethoxy, hydroxyl, (C₁-C₄)-alkoxy, benzyloxy, phenoxy and phenyl, and up to hexasubstituted by fluorine,
      in which benzyloxy, phenoxy and phenyl may be substituted by 1 to 3 halogen substituents,
   in which (C₃-C₇)-cycloalkyl may be substituted by 1 or 2 fluorine or (C₁-C₄)-alkyl substituents,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R⁹ is hydrogen or (C₁-C₆)-alkyl,
or
R⁸ and R⁹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
or
R⁶ and R⁸ together with the carbon atoms to which they are bonded form a 3- to 7-membered carbocycle or a 4- to 7-membered heterocycle,
with the proviso that not more than one of the R⁶ and R⁷, R⁸ and R⁹, and R⁶ and R⁸ radical pairs at the same time forms a carbo- or heterocycle,
with the proviso that the R⁶ and R⁸ radicals are not both simultaneously phenyl or 5- or 6-membered heteroaryl,
R¹⁰ is hydrogen or (C₁-C₄)-alkyl,
   in which (C₁-C₄)-alkyl may be substituted by 1 to 3 substituents selected independently from the group of fluorine, trifluoromethyl, hydroxyl and (C₁-C₄)-alkoxy,
R¹¹ is hydrogen, (C₁-C₆)-alkyl, (C₃-C₇)-cycloalkyl, phenyl or benzyl,
   in which (C₁-C₆)-alkyl may be substituted by 1 to 3 substituents selected independently from the group of fluorine, trifluoromethyl, hydroxyl, (C₁-C₄)-alkoxy and phenoxy,
   and
   in which phenyl and benzyl may be substituted by 1 to 3 substituents selected independently from the group of halogen and trifluoromethyl,
or
R¹⁰ and R¹¹ together with the nitrogen atom to which they are bonded form a 4- to 7-membered azaheterocycle,
R¹² is 5- to 10-membered azaheterocyclyl bonded via a ring carbon atom,
   in which 5- to 10-membered azaheterocyclyl bonded via a ring carbon atom may be substituted by 1 or 2 substituents selected independently from the group of trifluoromethyl, (C₃-C₇)-cycloalkyl, oxo and benzyl, and up to tetrasubstituted by (C₁-C₄)-alkyl and up to disubstituted by fluorine,
   and
   in which 5- to 10-membered azaheterocyclyl bonded via a ring carbon atom may be fused to a phenyl ring which may in turn be substituted by 1 or 2 substituents selected from halogen, (C₁-C₄)-alkyl and trifluoromethyl,
   or
   may be amino when L² is a bond,
   in which amino may be substituted by (C₁-C₁₀)-alkyl, (C₁-C₄)-alkylcarbonyl, (C₃-C₆)-carbocyclyl, 4- to 7-membered heterocyclyl, phenyl or 5- or 6-membered heteroaryl,
      in which (C₁-C₄)-alkylcarbonyl may be substituted by monoalkylamino or dialkylamino,
      in which (C₃-C₆)-carbocyclyl and 4- to 7-membered heterocyclyl may be substituted by hydroxyl,
      and
      in which phenyl and 5- or 6-membered heteroaryl may be substituted by 1 to 3 substituents selected independently from the group of halogen, (C₁-C₄)-alkyl and trifluoromethyl,
R¹³ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, —(C═O)NR²³R²⁴, 5- or 6-membered heteroaryl or phenyl,
   in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
      in which phenyl, phenoxy and benzyloxy may in turn be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   in which R²³ is hydrogen, (C₁-C₄)-alkyl, (C₃-C₇)-cycloalkyl, aryl or naphthyl,
   in which R²⁴ is hydrogen or methyl,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁴ is hydrogen or (C₁-C₆)-alkyl,
   in which (C₁-C₄)-alkyl may be substituted by hydroxyl,
or
R¹³ and R¹⁴ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
R¹⁵ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, 5- or 6-membered heteroaryl or phenyl,
   in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
      in which phenyl, phenoxy and benzyloxy may in turn be substituted by 1 to 3 halogen substituents,
   in which (C₃-C₇)-cycloalkyl may be substituted by 1 or 2 substituents selected independently from the group of fluorine, trifluoromethyl, (C₁-C₄)-alkyl and (C₁-C₄)-alkoxy,
   and
   in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁶ is hydrogen or (C₁-C₆)-alkyl,
   in which (C₁-C₄)-alkyl may be substituted by hydroxyl,
or
R¹⁵ and R¹⁶ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
with the proviso that the R¹³ and R¹⁵ radicals are not both simultaneously phenyl or 5- or 6-membered heteroaryl,
or
R¹³ and R¹⁵ together with the carbon atoms to which they are bonded form a 3- to 7-membered carbocycle or a 4- to 7-membered heterocycle,
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
   in which (C₁-C₄)-alkyl may be substituted by 1 to 5 fluorine substituents,
m is 0, 1 or 2,
n is 0 or 1,
R¹⁸ is hydrogen, cyano or (C₁-C₄)-alkyl,
   in which (C₁-C₄)-alkyl may be substituted by 1 to 5 fluorine substituents,
R¹⁹ is hydrogen or (C₁-C₄)-alkyl,
   in which (C₁-C₄)-alkyl may be substituted by 1 to 5 fluorine substituents,
R²⁰ is hydrogen or (C₁-C₄)-alkyl,
   in which (C₁-C₄)-alkyl may be substituted by 1 to 5 fluorine substituents,
R²¹ is hydrogen or (C₁-C₄)-alkyl,
   in which (C₁-C₄)-alkyl may be substituted by 1 to 3 fluorine substituents,
or
R¹⁸ and R¹⁹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
or
R²⁰ and R²¹ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
or
R¹⁸ and R²⁰ together with the carbon atom to which they are bonded form a 3- to 7-membered carbocycle or a 4- to 7-membered heterocycle,
   in which the 3- to 7-membered carbocycle and the 4- to 7-membered heterocycle may in turn be substituted by 1 or 2 substituents selected independently from the group of fluorine and (C₁-C₄)-alkyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, cyano, (C₁-C₆)-alkoxy, 5- to 9-membered heterocyclyl bonded via a ring carbon atom, 5- to 9-membered carbocyclyl, phenyl, indanyl or 5- to 10-membered heteroaryl,
   in which (C₁-C₆)-alkyl may be substituted by cyano and up to hexasubstituted by fluorine,
   in which (C₁-C₆)-alkoxy may be substituted by hydroxyl, amino, monoalkylamino, dialkylamino, cyclopropyl, phenyl or (C₂-C₄)-alkenyl,
   in which phenyl may be substituted by 1 to 3 substituents independently of one another selected from the group consisting of halogen, cyano, trifluoromethyl, difluoromethyl, (C₁-C₆)-alkyl, (C₁-C₄)-alkylcarbonyl, (C₁-C₄)-alkoxycarbonyl, hydroxycarbonyl, —(C═O)NR²⁵R²⁶, (C₁-C₄)-alkylsulphonyl, (C₃-C₆)-cycloalkylsulphonyl, (C₁-C₄)-alkylthio, (C₁-C₄)-alkoxy, trifluoromethoxy, difluoromethoxy, phenoxy, hydroxyl, 5- to 10-membered heteroaryl and (C₃-C₇)-cycloalkyl,
      in which (C₁-C₆)-alkyl may be substituted by 1 or 2 substituents selected independently from the group consisting of fluorine, trifluoromethoxy, (C₁-C₄)-alkylcarbonyl, —(C═O)NR²⁵R²⁶, (C₁-C₄)-alkoxy, (C₃-C₆)-cycloalkyl, morpholinyl, piperidinyl, pyrrolidinyl, piperazinyl, phenyl, hydroxyl and amino,
         in which phenyl may be substituted by 1 to 3 halogen substituents,
         in which amino may be substituted by 1 or 2 substituents selected independently from (C₁-C₆)-alkyl, (C₁-C₄)-alkylcarbonyl, (C₃-C₆)-cycloalkylsulphonyl, (C₁-C₄)-alkylsulphonyl and methoxy-(C₁-C₄)-alkyl,
         in which (C₃-C₆)-cycloalkyl may be substituted by amino or hydroxyl,
         and in which
         R²⁵ and R²⁶ are each independently hydrogen, (C₁-C₄)-alkyl or (C₃-C₇)-cycloalkyl,
   in which indanyl may be substituted by 1 or 2 substituents selected independently from the group of fluorine, trifluoromethyl and hydroxyl,
   in which 5- to 10-membered heteroaryl may be substituted by 1 to 3 substituents independently of one another selected from the group of fluorine, chlorine, cyano, (C₁-C₆)-alkyl, trifluoromethyl, (C₁-C₄)-alkoxy, amino, (C₁-C₄)-alkoxycarbonyl, hydroxycarbonyl, —(C═O)NR²⁵R²⁶, phenyl, pyridyl, pyrimidyl, 1,3-thiazol-5-yl and (C₃-C₇)-cycloalkyl,
      in which (C₁-C₆)-alkyl may be substituted by 1 to 3 substituents selected independently from the group of halogen, cyano, hydroxyl, amino, trifluoromethyl, difluoromethyl, (C₁-C₄)-alkylsulphonyl, (C₁-C₄)-alkylcarbonyl, (C₁-C₄)-alkoxycarbonyl, hydroxycarbonyl, (C₁-C₄)-alkylthio, (C₁-C₄)-alkoxy, trifluoromethoxy, difluoromethoxy, phenoxy, phenyl, pyridyl, pyrimidyl, 5-membered heteroaryl, tetrahydrothiophenyl 1,1-dioxide, (C₃-C₇)-cycloalkyl, morpholinyl, piperidinyl, pyrrolidinyl, 2-oxopyrrolidin-1-yl, piperazinyl, tetrahydrothiophenyl 1,1-dioxide, thiomorpholinyl 1,1-dioxide and azetidine,
      in which 5-membered heteroaryl may be substituted by 1 to 3 substituents selected independently from the group of halogen, (C₁-C₄)-alkyl and (C₁-C₄)-alkoxy,
      in which piperidinyl may be substituted by 1 to 4 fluorine substituents,
      in which phenyl may be substituted by 1 to 3 substituents selected independently from the group of halogen, (C₁-C₄)-alkyl and (C₁-C₄)-alkoxy,
      in which azetidine may be substituted by hydroxyl,
      in which piperazinyl may be substituted by 1 to 3 substituents selected independently from the group consisting of (C₁-C₄)-alkyl, (C₃-C₇)-cycloalkyl and trifluoromethyl,
      and in which
      R²⁵ and R²⁶ are each independently hydrogen, (C₁-C₄)-alkyl or (C₃-C₇)-cycloalkyl,
   in which 5- to 9-membered heterocyclyl bonded via a ring carbon atom may be substituted by 1 or 2 substituents selected independently from the group of oxo, fluorine, hydroxyl and (C₁-C₄)-alkyl,
   and
   in which 5- to 9-membered carbocyclyl may be substituted by 1 or 2 substituents selected independently from the group of trifluoromethyl, fluorine, cyano, hydroxyl, hydroxycarbonyl, (C₁-C₄)-alkoxycarbonyl and (C₁-C₄)-alkyl,
R⁴ is hydrogen,
R⁵ is hydrogen, halogen, cyano, difluoromethyl, trifluoromethyl, (C₁-C₄)-alkyl, (C₃-C₇)-cycloalkyl, (C₂-C₄)-alkynyl, (C₁-C₄)-alkylamino, difluoromethoxy, trifluoromethoxy, (C₁-C₄)-alkoxy, amino, 4- to 7-membered heterocyclyl or 5- or 6-membered heteroaryl,
   and the N-oxides, salts, solvates, salts of the N-oxides and solvates of the N-oxides or salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by the formula (I) and are mentioned below as embodiments and the salts, solvates and solvates of the salts thereof if the compounds that are encompassed by the formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds of the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, of conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

The present invention additionally also encompasses prodrugs of the compounds of the invention. The term “prodrugs” in this context refers to compounds which may themselves be biologically active or inactive but are converted (for example metabolically or hydrolytically) to compounds of the invention during their residence time in the body.

In the context of the present invention, unless specified otherwise, the substituents are defined as follows:

Alkyl in the context of the invention is a straight-chain or branched alkyl radical having the particular number of carbon atoms specified. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl.

Cycloalkyl or carbocycle or carbocyclyl in the context of the invention is a monocyclic saturated alkyl radical having the particular number of ring carbon atoms specified. The following may be mentioned by way of example and by way of preference: cyclopropyl, cyclobutyl, cyclopentyl, cyclohexyl and cycloheptyl.

Alkenyl in the context of the invention is a linear or branched alkenyl radical having 2 to 6 carbon atoms and one or two double bonds. Preference is given to a linear or branched alkenyl radical having 2 to 4 carbon atoms and one double bond. The following may be mentioned by way of example and by way of preference: vinyl, allyl, isopropenyl and n-but-2-en-1-yl.

Alkynyl in the context of the invention is a straight-chain or branched alkynyl radical having 2 to 6 carbon atoms and one triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkanediyl in the context of the invention is a linear or branched divalent alkyl radical having 1 to 4 carbon atoms. The following may be mentioned by way of example and by way of preference: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl and butane-2,3-diyl.

Alkoxy in the context of the invention is a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy and tert-butoxy.

Alkoxycarbonyl in the context of the invention is a straight-chain or branched alkoxy radical having 1 to 4 carbon atoms and a carbonyl group attached to the oxygen atom. The following may be mentioned by way of example and by way of preference: methoxycarbonyl, ethoxycarbonyl, n-propoxycarbonyl, isopropoxycarbonyl and tert-butoxycarbonyl.

Alkylsulphonyl in the context of the invention is a straight-chain or branched alkyl radical which has 1 to 4 carbon atoms and is bonded via a sulphonyl group. Preferred examples include: methylsulphonyl, ethylsulphonyl, n-propylsulphonyl, isopropylsulphonyl, n-butylsulphonyl and tert-butylsulphonyl.

A 4- to 7-membered heterocycle in the context of the invention is a monocyclic saturated heterocycle which has a total of 4 to 7 ring atoms, contains one or two ring heteroatoms from the group consisting of N, O, S, SO and SO₂ and is joined via a ring carbon atom or any ring nitrogen atom. Examples include: azetidinyl, oxetanyl, pyrrolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to azetidinyl, oxetanyl, pyrrolidinyl, tetrahydrofuranyl, piperidinyl, piperazinyl, tetrahydropyranyl and morpholinyl.

A 4- to 7-membered azaheterocycle in the context of the invention is a monocyclic saturated heterocycle which has a total of 4 to 7 ring atoms, contains a nitrogen atom and may additionally contain a further ring heteroatom from the group of N, O, S, SO and SO₂, and is joined via a ring nitrogen atom. Examples include: azetidinyl, pyrrolidinyl, pyrazolidinyl, piperidinyl, piperazinyl, morpholinyl, thiomorpholinyl, 1,1-dioxothiomorpholinyl, hexahydroazepinyl and hexahydro-1,4-diazepinyl.

5- to 9-membered azaheterocyclyl in the context of the invention is a monocyclic or bicyclic, saturated or partly unsaturated heterocycle which has a total of 5 to 9 ring atoms, contains a nitrogen atom and may additionally contain one or two further ring heteroatom(s) from the group of N, O, S, SO and/or SO₂, and is joined via a ring carbon atom. Examples include: pyrrolidinyl, pyrazolidinyl, piperidinyl, piperazinyl, morpholinyl, thiomorpholinyl, 1,1-dioxothiomorpholinyl, hexahydroazepinyl, hexahydro-1,4-diazepinyl, 1,2,3,4-tetrahydroisoquinolinyl, 1,2,3,4-tetrahydroquinolinyl, indolinyl, 8-azabicyclo[3.2.1]octanyl, 9-azabicyclo[3.3.1]nonanyl, 3-azabicyclo[4.1.0]heptanyl and quinuclidinyl.

Heteroaryl in the context of the invention is a monocyclic aromatic heterocycle (heteroaromatic) which has a total of 5 or 6 ring atoms, contains up to three identical or different ring heteroatoms from the group of N, O and/or S and is joined via a ring carbon atom or via any ring nitrogen atom. The following may be mentioned by way of example and by way of preference: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl.

Halogen in the context of the invention includes fluorine, chlorine, bromine and iodine. Preference is given to chlorine or fluorine.

In the formula of the group that R³ or R¹ may represent, the end point of the line marked by the symbol * and # does not represent a carbon atom or a CH₂ group but is part of the bond to the respective atom to which R³ or R¹ is bonded.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is used here synonymously with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, preference is given to compounds of the formula (I) in which
A is CH₂ or CD₂,
R¹ is (C₃-C₆)-cycloalkyl, pyridyl or phenyl,
   where (C₃-C₆)-cycloalkyl may be substituted by 1 to 2 substituents selected independently from the group of fluorine, trifluoromethyl, methyl and ethyl,
   where pyridyl is substituted by 1 or 2 F substituents,
   and
   where phenyl may be substituted by 1 to 4 substituents selected independently from the group of halogen, cyano, difluoromethyl, trifluoromethyl, (C₁-C₄)-alkyl, (C₁-C₄)-alkoxy and (C₃-C₅)-cyclopropyl,
R² is hydrogen, (C₁-C₄)-alkyl, cyclopropyl, difluoromethyl or trifluoromethyl,
R³ is a group of the formula

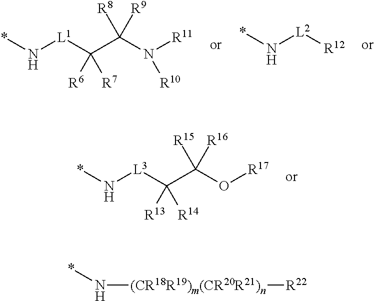

where
* is the attachment site to the carbonyl group,
L¹ is a bond, methanediyl or 1,2-ethanediyl,
L² is a bond, methanediyl or 1,2-ethanediyl,
L³ is a bond, methanediyl or 1,2-ethanediyl,
R⁶ is hydrogen, (C₁-C₆)-alkyl, (C₃-C₇)-cycloalkyl, phenyl or 5- or 6-membered heteroaryl,
   in which (C₁-C₆)-alkyl may be substituted by 1 to 5 fluorine substituents,
   and
   in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁷ is hydrogen or (C₁-C₄)-alkyl,
or
R⁶ and R⁷ together with the carbon atom to which they are bonded form a 3- to 5-membered carbocycle,
R⁸ is hydrogen, (C₁-C₆)-alkyl, (C₃-C₅)-cycloalkyl, 5- or 6-membered heteroaryl or phenyl,
   in which (C₁-C₆)-alkyl may be substituted by 1 to 5 fluorine substituents,
   in which (C₁-C₆)-alkyl may be substituted by (C₁-C₄)-alkoxy, benzyloxy or phenoxy,
      in which benzyloxy and phenoxy may be substituted by 1 to 3 substituents selected independently from the group of fluorine, chlorine and bromine,
   in which (C₃-C₅)-cycloalkyl may be substituted by 1 or 2 fluorine or (C₁-C₄)-alkyl substituents,
   and
   in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁹ is hydrogen or (C₁-C₄)-alkyl,
or
R⁸ and R⁹ together with the carbon atom to which they are bonded form a 3- to 5-membered carbocycle,
or
R⁶ and R⁸ together with the carbon atom to which they are bonded form a 3- to 6-membered carbocycle or a 4- to 7-membered heterocycle,
   in which the 3- to 6-membered carbocycle and the 4- to 7-membered heterocycle may be substituted by 1 or 2 fluorine or (C₁-C₄)-alkyl substituents,
with the proviso that not more than one of the R⁶ and R⁷, R⁸ and R⁹, and R⁶ and R⁸ radical pairs at the same time forms a carbo- or heterocycle,
and
with the proviso that the R⁶ and R⁸ radicals are not both simultaneously phenyl or 5- or 6-membered heteroaryl,
R¹⁰ is hydrogen or (C₁-C₄)-alkyl,
   in which (C₁-C₄)-alkyl may be substituted by 1 to 5 fluorine substituents,
R¹¹ is hydrogen, (C₁-C₆)-alkyl or (C₃-C₇)-cycloalkyl,
   in which (C₁-C₆)-alkyl may be substituted by 1 to 5 fluorine substituents,
or
R¹⁰ and R¹¹ together with the nitrogen atom to which they are bonded form a 4- to 7-membered azaheterocycle,
R¹² is 5- to 9-membered azaheterocyclyl bonded via a ring carbon atom,
   in which 5- to 9-membered azaheterocyclyl may be substituted by 1 to 5 substituents selected independently from the group of fluorine, methyl and ethyl,
R¹³ is hydrogen, (C₁-C₆)-alkyl, (C₃-C₅)-cycloalkyl, —(C═O)NR²³R²⁴, 5- or 6-membered heteroaryl or phenyl,
   in which (C₁-C₆)-alkyl may be substituted by 1 to 3 substituents each independently selected from the group of fluorine, trifluoromethyl, difluoromethoxy, trifluoromethoxy, hydroxyl and (C₁-C₄)-alkoxy,
   in which R²³ is hydrogen, (C₁-C₄)-alkyl, aryl or naphthyl,
   in which R²⁴ is hydrogen,
   and
   in which phenyl and 5- or 6-membered heteroaryl may be substituted by 1 to 3 substituents selected independently from the group of fluorine, chlorine, bromine, trifluoromethyl, methyl and ethyl,
R¹⁴ is hydrogen or (C₁-C₄)-alkyl,
or
R¹³ and R¹⁴ together with the carbon atom to which they are bonded form a 3- to 5-membered carbocycle,
R¹⁵ is hydrogen, (C₁-C₆)-alkyl or (C₃-C₅)-cycloalkyl,
   in which (C₁-C₆)-alkyl may be substituted by 1 to 5 fluorine substituents,
   and
   in which (C₃-C₅)-cycloalkyl may be substituted by 1 or 2 substituents selected independently from the group consisting of fluorine, trifluoromethyl, hydroxyl and (C₁-C₄)-alkyl,
R¹⁶ is hydrogen or (C₁-C₄)-alkyl,
or
R¹⁵ and R¹⁶ together with the carbon atom to which they are bonded form a 3- to 6-membered carbocycle,
   in which the 3- to 6-membered carbocycle may be substituted by 1 or 2 fluorine or (C₁-C₄)-alkyl substituents,
or
R¹³ and R¹⁵ together with the carbon atom to which they are bonded form a 3- to 6-membered carbocycle or a 4- to 7-membered heterocycle,
   in which the 3- to 6-membered carbocycle and the 4- to 7-membered heterocycle may be substituted by 1 or 2 fluorine or (C₁-C₄)-alkyl substituents,
with the proviso that the R¹³ and R¹⁵ radicals are not both simultaneously phenyl,
and
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
   in which (C₁-C₄)-alkyl may be substituted by 1 to 5 fluorine substituents,
m is 0 or 1,
n is 0 or 1,
R¹⁸ is hydrogen, cyano or (C₁-C₄)-alkyl,
   in which (C₁-C₄)-alkyl may be substituted by 1 to 5 fluorine substituents,
R¹⁹ is hydrogen or (C₁-C₄)-alkyl,
   in which (C₁-C₄)-alkyl may be substituted by 1 to 5 fluorine substituents,
R²⁰ is hydrogen or (C₁-C₄)-alkyl,
   in which (C₁-C₄)-alkyl may be substituted by 1 to 5 fluorine substituents,
R²¹ is hydrogen or (C₁-C₄)-alkyl,
   in which (C₁-C₄)-alkyl may be substituted by 1 to 5 fluorine substituents,
or
R¹⁸ and R¹⁹ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
or
R²⁰ and R²¹ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
or
R¹⁸ and R²⁰ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, 5- to 9-membered heterocyclyl bonded via a ring carbon atom, 5- to 9-membered carbocyclyl, phenyl, indanyl or 5- to 10-membered heteroaryl,
   where (C₁-C₆)-alkyl may be substituted by cyano or up to three times by fluorine,
   where phenyl may be substituted by 1 to 3 substituents selected independently from the group of halogen, cyano, trifluoromethyl, difluoromethyl, (C₁-C₄)-alkyl, (C₁-C₄)-alkoxy and 5- to 10-membered heteroaryl,
      in which (C₁-C₄)-alkyl may be substituted by 1 or 2 substituents selected independently from the group of fluorine, trifluoromethoxy, (C₁-C₄)-alkoxy, (C₃-C₆)-cycloalkyl, hydroxyl and amino,
   where indanyl may be substituted by 1 or 2 substituents selected independently from the group of fluorine, trifluoromethyl and hydroxyl,
   where 5- to 10-membered heteroaryl may be substituted by 1 to 3 substituents selected independently from the group of fluorine, chlorine, cyano, trifluoromethyl, (C₁-C₄)-alkyl, (C₁-C₄)-alkoxy, amino and hydroxyl,
      in which (C₁-C₄)-alkyl may be substituted by 1 to 3 substituents selected independently from the group of halogen, cyano, hydroxyl, amino, trifluoromethyl, difluoromethyl, (C₁-C₄)-alkoxy, trifluoromethoxy, difluoromethoxy and phenyl,
         in which phenyl may be substituted by 1 to 3 halogen substituents,
   where 5- to 9-membered heterocyclyl bonded via a ring carbon atom may be substituted by 1 or 2 substituents selected independently from the group of oxo, fluorine, hydroxyl and (C₁-C₄)-alkyl,
   and
   where 5- to 9-membered carbocyclyl may be substituted by 1 or 2 substituents selected independently from the group of trifluoromethyl, fluorine, hydroxyl, hydroxycarbonyl, (C₁-C₄)-alkoxycarbonyl and (C₁-C₄)-alkyl,
R⁴ is hydrogen,
R⁵ is hydrogen, fluorine, chlorine, bromine, cyano, difluoromethyl, trifluoromethyl, (C₁-C₄)-alkyl, (C₂-C₄)-alkynyl or (C₃-C₅)-cycloalkyl,
   and the N-oxides, salts, solvates, salts of the N-oxides and solvates of the N-oxides or salts thereof.

In the context of the present invention, preference is given to compounds of the formula (I) in which
A is CH₂ or CD₂,
R¹ is (C₃-C₆)-cycloalkyl, pyridyl or phenyl,
   where (C₃-C₆)-cycloalkyl may be substituted by 1 to 2 substituents selected independently from the group of fluorine, trifluoromethyl, methyl and ethyl,
   where pyridyl is substituted by 1 or 2 fluorine substituents,
   and
   where phenyl may be substituted by 1 to 4 substituents selected independently from the group of halogen, cyano, difluoromethyl, trifluoromethyl, (C₁-C₄)-alkyl, (C₁-C₄)-alkoxy and (C₃-C₅)-cyclopropyl,
R² is hydrogen, (C₁-C₄)-alkyl, cyclopropyl, difluoromethyl or trifluoromethyl,
R³ is a group of the formula

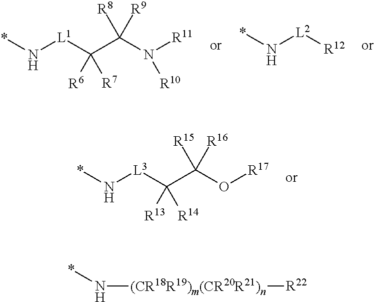

where
* is the attachment site to the carbonyl group,
L¹ is a bond, methanediyl or 1,2-ethanediyl,
L² is a bond, methanediyl or 1,2-ethanediyl,
L³ is a bond, methanediyl or 1,2-ethanediyl,
R⁶ is hydrogen, (C₁-C₆)-alkyl, (C₃-C₇)-cycloalkyl, phenyl or 5- or 6-membered heteroaryl,
   in which (C₁-C₆)-alkyl may be substituted up to five times by fluorine,
   and
   in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁷ is hydrogen or (C₁-C₄)-alkyl,
or
R⁶ and R⁷ together with the carbon atom to which they are bonded form a 3- to 5-membered carbocycle,
R⁸ is hydrogen, (C₁-C₆)-alkyl, (C₃-C₅)-cycloalkyl, 5- or 6-membered heteroaryl or phenyl,
   in which (C₁-C₆)-alkyl may be substituted by (C₁-C₄)-alkoxy, benzyloxy or phenoxy, and up to pentasubstituted by fluorine,
      in which benzyloxy and phenoxy may be substituted by 1 to 3 substituents selected independently from the group of fluorine, chlorine and bromine,
   in which (C₃-C₅)-cycloalkyl may be substituted by 1 or 2 fluorine or (C₁-C₄)-alkyl substituents,
   and
   in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁹ is hydrogen or (C₁-C₄)-alkyl,
or
R⁸ and R⁹ together with the carbon atom to which they are bonded form a 3- to 5-membered carbocycle,
or
R⁶ and R⁸ together with the carbon atom to which they are bonded form a 3- to 6-membered carbocycle or a 4- to 7-membered heterocycle,
   in which the 3- to 6-membered carbocycle and the 4- to 7-membered heterocycle may be substituted by 1 or 2 fluorine or (C₁-C₄)-alkyl substituents,
with the proviso that not more than one of the R⁶ and R⁷, R⁸ and R⁹, and R⁶ and R⁸ radical pairs at the same time forms a carbo- or heterocycle,
and
with the proviso that the R⁶ and R⁸ radicals are not both simultaneously phenyl or 5- or 6-membered heteroaryl,
R¹⁰ is hydrogen or (C₁-C₄)-alkyl,
   in which (C₁-C₄)-alkyl may be substituted up to five times by fluorine,
R¹¹ is hydrogen, (C₁-C₆)-alkyl or (C₃-C₇)-cycloalkyl,
   in which (C₁-C₆)-alkyl may be substituted up to five times by fluorine,
or
R¹⁰ and R¹¹ together with the nitrogen atom to which they are bonded form a 4- to 7-membered azaheterocycle,
R¹² is 5- to 10-membered azaheterocyclyl bonded via a ring carbon atom,
   in which 5- to 10-membered azaheterocyclyl may be substituted by 1 to 5 substituents selected independently from the group of fluorine, methyl and ethyl,
   or
   may be amino when L² is a bond,
   in which amino may be substituted by (C₁-C₄)-alkyl, (C₁-C₄)-alkylcarbonyl, (C₃-C₆)-carbocyclyl, 4- to 7-membered heterocyclyl, phenyl or 5- or 6-membered heteroaryl,
      in which (C₁-C₄)-alkylcarbonyl may be substituted by monoalkylamino or dialkylamino,
      in which (C₃-C₆)-carbocyclyl and 4- to 7-membered heterocyclyl may be substituted by hydroxyl,
      and
      in which phenyl and 5- or 6-membered heteroaryl may be substituted by 1 to 3 substituents selected independently from the group of fluorine, chlorine, methyl and trifluoromethyl,
R¹³ is hydrogen, (C₁-C₆)-alkyl, (C₃-C₅)-cycloalkyl, —(C═O)NR²³R²⁴, 5- or 6-membered heteroaryl or phenyl,
   in which (C₁-C₆)-alkyl may be substituted by 1 or 2 substituents each independently selected from the group of difluoromethoxy, trifluoromethoxy, hydroxyl and (C₁-C₄)-alkoxy, and up to hexasubstituted by fluorine,
   in which R²³ is hydrogen, (C₁-C₄)-alkyl, aryl or naphthyl,
   in which R²⁴ is hydrogen,
   and
   in which phenyl and 5- or 6-membered heteroaryl may be substituted by 1 to 3 substituents selected independently from the group of fluorine, chlorine, bromine, trifluoromethyl, methyl and ethyl,
R¹⁴ is hydrogen or (C₁-C₄)-alkyl,
or
R¹³ and R¹⁴ together with the carbon atom to which they are bonded form a 3- to 5-membered carbocycle,
R¹⁵ is hydrogen, (C₁-C₆)-alkyl or (C₃-C₅)-cycloalkyl,
   in which (C₁-C₆)-alkyl may be substituted up to five times by fluorine,
   and
   in which (C₃-C₅)-cycloalkyl may be substituted by 1 or 2 substituents selected independently from the group consisting of fluorine, trifluoromethyl, hydroxyl and (C₁-C₄)-alkyl,
R¹⁶ is hydrogen or (C₁-C₄)-alkyl,
or
R¹⁵ and R¹⁶ together with the carbon atom to which they are bonded form a 3- to 6-membered carbocycle,
   in which the 3- to 6-membered carbocycle may be substituted by 1 or 2 fluorine or (C₁-C₄)-alkyl substituents,
or
R¹³ and R¹⁵ together with the carbon atom to which they are bonded form a 3- to 6-membered carbocycle or a 4- to 7-membered heterocycle,
   in which the 3- to 6-membered carbocycle and the 4- to 7-membered heterocycle may be substituted by 1 or 2 fluorine or (C₁-C₄)-alkyl substituents,
with the proviso that the R¹³ and R¹⁵ radicals are not both simultaneously phenyl,
and
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
   in which (C₁-C₄)-alkyl may be substituted by 1 to 5 fluorine substituents,
m is 0 or 1,
n is 0 or 1,
R¹⁸ is hydrogen, cyano or (C₁-C₄)-alkyl,
   in which (C₁-C₄)-alkyl may be substituted by 1 to 5 fluorine substituents,
R¹⁹ is hydrogen or (C₁-C₄)-alkyl,
   in which (C₁-C₄)-alkyl may be substituted by 1 to 5 fluorine substituents,
R²⁰ is hydrogen or (C₁-C₄)-alkyl,
   in which (C₁-C₄)-alkyl may be substituted by 1 to 5 fluorine substituents,
R²¹ is hydrogen or (C₁-C₄)-alkyl,
   in which (C₁-C₄)-alkyl may be substituted by 1 to 5 fluorine substituents,
or
R¹⁸ and R¹⁹ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
or
R²⁰ and R²¹ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
or
R¹⁸ and R²⁰ together with the carbon atom to which they are bonded form a 3- to 5-membered carbocycle,
   in which the 3- to 5-membered carbocycle may be substituted by 1 or 2 substituents selected independently from the group of fluorine, methyl and ethyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, cyano, (C₁-C₆)-alkoxy, 5- to 9-membered heterocyclyl bonded via a ring carbon atom, 5- to 9-membered carbocyclyl, phenyl, indanyl or 5- to 10-membered heteroaryl,
   in which (C₁-C₆)-alkyl may be substituted by cyano or up to pentasubstituted by fluorine,
   in which (C₁-C₆)-alkoxy may be substituted by hydroxyl or (C₂-C₄)-alkenyl,
   in which phenyl may be substituted by 1 to 3 substituents selected independently from the group of halogen, cyano, trifluoromethyl, difluoromethyl, (C₁-C₄)-alkyl, (C₁-C₄)-alkoxy and 5- to 10-membered heteroaryl,
      in which (C₁-C₄)-alkyl may be substituted by 1 or 2 substituents selected independently from the group of fluorine, trifluoromethoxy, (C₁-C₄)-alkoxy, (C₃-C₆)-cycloalkyl, hydroxyl and amino,
   in which indanyl may be substituted by 1 or 2 substituents selected independently from the group of fluorine, trifluoromethyl and hydroxyl,
   in which 5- to 10-membered heteroaryl may be substituted by 1 to 3 substituents selected independently from the group of fluorine, chlorine, cyano, trifluoromethyl, (C₁-C₄)-alkyl, (C₁-C₄)-alkoxy, amino and hydroxyl,
      in which (C₁-C₄)-alkyl may be substituted by 1 to 3 substituents selected independently from the group of halogen, cyano, hydroxyl, amino, trifluoromethyl, difluoromethyl, (C₁-C₄)-alkoxy, trifluoromethoxy, difluoromethoxy and phenyl,
         in which phenyl may be substituted by 1 to 3 halogen substituents,
   in which 5- to 9-membered heterocyclyl bonded via a ring carbon atom may be substituted by 1 or 2 substituents selected independently from the group of oxo, fluorine, hydroxyl and (C₁-C₄)-alkyl,
   and
   in which 5- to 9-membered carbocyclyl may be substituted by 1 or 2 substituents selected independently from the group of trifluoromethyl, fluorine, hydroxyl, hydroxycarbonyl, (C₁-C₄)-alkoxycarbonyl and (C₁-C₄)-alkyl,
R⁴ is hydrogen,
R⁵ is hydrogen, fluorine, chlorine, bromine, cyano, difluoromethyl, trifluoromethyl, (C₁-C₄)-alkyl, (C₂-C₄)-alkynyl or (C₃-C₅)-cycloalkyl,
   and the N-oxides, salts, solvates, salts of the N-oxides and solvates of the N-oxides or salts thereof.

In the context of the present invention, preference is given to compounds of the formula (I) in which
A is CH₂,
R¹ is cyclohexyl, pyridyl or phenyl,
   where cyclohexyl may be substituted by 1 to 2 substituents selected independently from the group of fluorine and methyl,
   where pyridyl is substituted by 1 or 2 fluorine substituents,
   and
   where phenyl may be substituted by 1 to 4 substituents selected independently from the group of fluorine, chlorine, methyl, methoxy and cyclopropyl,
R² is methyl, cyclopropyl or trifluoromethyl,
R³ is a group of the formula

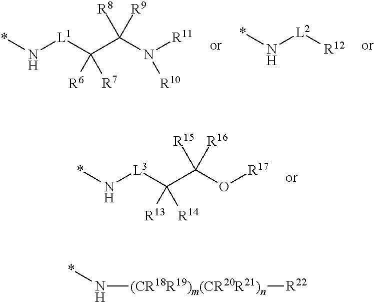

where
* is the attachment site to the carbonyl group,
L¹ is a bond,
L² is a bond,
L³ is a bond,
R⁶ is hydrogen, (C₁-C₆)-alkyl or phenyl,
   in which (C₁-C₆)-alkyl may be substituted by 1 to 5 fluorine substituents,
   and
   in which phenyl may be substituted by 1 to 3 substituents selected independently from the group of fluorine, chlorine, trifluoromethyl, methyl and methoxy,
R⁷ is hydrogen, methyl or ethyl,
R⁸ is hydrogen, (C₁-C₆)-alkyl, cyclopropyl or cyclobutyl,
   in which (C₁-C₆)-alkyl may be substituted by 1 to 5 fluorine substituents,
R⁹ is hydrogen or (C₁-C₄)-alkyl,
or
R⁸ and R⁹ together with the carbon atom to which they are bonded form a 3- to 6-membered carbocycle,
R¹⁰ is hydrogen, methyl or ethyl,
   in which ethyl may be substituted by 1 to 3 fluorine substituents,
R¹¹ is hydrogen, (C₁-C₄)-alkyl or (C₃-C₅)-cycloalkyl,
or
R¹⁰ and R¹¹ together with the nitrogen atom to which they are bonded form a morpholinyl ring or piperidinyl ring,
R¹² is 9-azabicyclo[3.3.1]nonan-3-yl or piperidin-4-yl,
   in which 9-azabicyclo[3.3.1]nonan-3-yl is substituted by methyl,
   in which piperidin-4-yl may be substituted by 1 to 5 methyl substituents,
R¹³ is hydrogen, (C₁-C₆)-alkyl, —(C═O)NR²³R²⁴ or phenyl,
   in which (C₁-C₆)-alkyl may be substituted by a hydroxyl or methoxy radical or up to five times by fluorine,
   in which R²³ is aryl or naphthyl,
   in which R²⁴ is hydrogen,
   and
   in which phenyl may be substituted by 1 to 3 substituents selected independently from the group consisting of fluorine, chlorine, trifluoromethyl and methyl,
R¹⁴ is hydrogen or (C₁-C₄)-alkyl,
R¹⁵ is hydrogen, (C₁-C₆)-alkyl, cyclopropyl or cyclobutyl,
   in which (C₁-C₆)-alkyl may be substituted by 1 to 5 fluorine substituents,
   in which cyclopropyl and cyclobutyl may be substituted by 1 or 2 substituents selected independently from the group of fluorine and methyl,
R¹⁶ is hydrogen or (C₁-C₄)-alkyl,
or
R¹⁵ and R¹⁶ together with the carbon atom to which they are bonded form a 3- to 6-membered carbocycle,
   in which the 3- to 6-membered carbocycle may be substituted by 1 or 2 fluorine or methyl substituents,
R¹⁷ is hydrogen or (C₁-C₄)-alkyl,
   in which (C₁-C₃)-alkyl may be substituted by 1 to 5 fluorine substituents,
m is 0 or 1,
n is 0 or 1,
R¹⁸ is hydrogen, cyano or methyl,
   in which methyl may be substituted by 1 to 3 fluorine substituents,
R¹⁹ is hydrogen or methyl,
   in which methyl may be substituted by 1 to 3 fluorine substituents,
R²⁰ si hydrogen or methyl,
   in which methyl may be substituted by 1 to 3 fluorine substituents,
R²¹ is hydrogen or methyl,
   in which methyl may be substituted by 1 to 3 fluorine substituents,
or
R¹⁸ and R¹⁹ together with the carbon atom to which they are bonded form a 3- to 5-membered carbocycle,
or
R¹⁸ and R²⁰ together with the carbon atom to which they are bonded form a cyclopropyl ring,
with the proviso that not more than one of the R¹⁸ and R¹⁹, and R¹⁸ and R²⁰ radical pairs at the same time forms a carbocycle,
R²² is (C₁-C₆)-alkyl, 2-oxopyrrolidin-3-yl, 2-oxotetrahydrofuran-3-yl, cyclopentyl, cyclohexyl, phenyl, indanyl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
   in which (C₁-C₆)-alkyl may be substituted by a cyano radical or up to trisubstituted by fluorine,
   where phenyl may be substituted by 1 to 3 substituents selected independently from the group of fluorine, chlorine, cyano, trifluoromethyl, methyl, ethyl, methoxy and pyridyl,
   where indanyl may be substituted by hydroxyl,
   where 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl may be substituted by 1 to 3 substituents selected independently from the group of fluorine, chlorine, trifluoromethyl, (C₁-C₃)-alkyl, amino and hydroxyl,
      in which (C₁-C₃)-alkyl may be substituted by fluorine, hydroxyl, amino or trifluoromethyl,
where cyclopentyl and cyclohexyl are substituted by methoxycarbonyl or ethoxycarbonyl,
R⁴ is hydrogen,
R⁵ is hydrogen, methyl or ethyl,
   and the N-oxides, salts, solvates, salts of the N-oxides and solvates of the N-oxides or salts thereof.

In the context of the present invention, preference is given to compounds of the formula (I) in which
A is CH₂,
R¹ is cyclohexyl, pyridyl or phenyl,
   where cyclohexyl may be substituted by 1 to 2 substituents selected independently from the group of fluorine and methyl,
   where pyridyl is substituted by 1 or 2 fluorine substituents,
   and
   where phenyl may be substituted by 1 to 4 substituents selected independently from the group of fluorine, chlorine, methyl, methoxy and cyclopropyl,
R² is methyl, cyclopropyl or trifluoromethyl,
R³ is a group of the formula

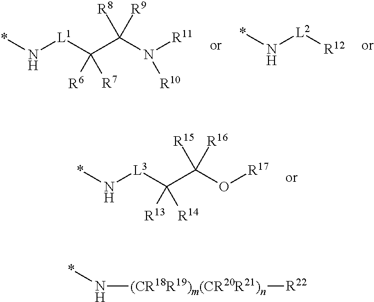

where
* is the attachment site to the carbonyl group,
L¹ is a bond,
L² is a bond,
L³ is a bond,
R⁶ is hydrogen, (C₁-C₆)-alkyl or phenyl,
   in which (C₁-C₆)-alkyl may be substituted up to five times by fluorine,
   and
   in which phenyl may be substituted by 1 to 3 substituents selected independently from the group of fluorine, chlorine, trifluoromethyl, methyl and methoxy,
R⁷ is hydrogen, methyl or ethyl,
R⁸ is hydrogen, (C₁-C₆)-alkyl, cyclopropyl or cyclobutyl,
   in which (C₁-C₆)-alkyl may be substituted up to five times by fluorine,
R⁹ is hydrogen or (C₁-C₄)-alkyl,
or
R⁸ and R⁹ together with the carbon atom to which they are bonded form a 3- to 6-membered carbocycle,
R¹⁰ is hydrogen, methyl or ethyl,
   in which ethyl may be substituted up to three times by fluorine,
R¹¹ is hydrogen, (C₁-C₄)-alkyl or (C₃-C₅)-cycloalkyl,
or
R¹⁰ and R¹¹ together with the nitrogen atom to which they are bonded form a morpholinyl ring or piperidinyl ring,
R¹² is 9-azabicyclo[3.3.1]nonan-3-yl or piperidin-4-yl,
   in which 9-azabicyclo[3.3.1]nonan-3-yl is substituted by methyl,
   in which piperidin-4-yl may be substituted by 1 to 5 methyl substituents,
R¹³ is hydrogen, (C₁-C₆)-alkyl, —(C═O)NR²³R²⁴ or phenyl,
   in which (C₁-C₆)-alkyl may be substituted by a hydroxyl or methoxy radical or up to five times by fluorine,
   in which R²³ is aryl or naphthyl,
   in which R²⁴ is hydrogen,
   and
   in which phenyl may be substituted by 1 to 3 substituents selected independently from the group consisting of fluorine, chlorine, trifluoromethyl and methyl,
R¹⁴ is hydrogen or (C₁-C₄)-alkyl,
R¹⁵ is hydrogen, (C₁-C₆)-alkyl, cyclopropyl or cyclobutyl,
   in which (C₁-C₆)-alkyl may be substituted up to five times by fluorine,
   in which cyclopropyl and cyclobutyl may be substituted by 1 or 2 substituents selected independently from the group of fluorine and methyl,
R¹⁶ is hydrogen or (C₁-C₄)-alkyl,
or
R¹⁵ and R¹⁶ together with the carbon atom to which they are bonded form a 3- to 6-membered carbocycle,
   in which the 3- to 6-membered carbocycle may be substituted by 1 or 2 fluorine or methyl substituents,
R¹⁷ is hydrogen or (C₁-C₄)-alkyl,
   in which (C₁-C₃)-alkyl may be substituted by 1 to 5 fluorine substituents,
m is 0 or 1,
n is 0 or 1,
R¹⁸ is hydrogen, cyano or methyl,
   in which methyl may be substituted by 1 to 3 fluorine substituents,
R¹⁹ is hydrogen or methyl,
   in which methyl may be substituted by 1 to 3 fluorine substituents,
R²⁰ is hydrogen or methyl,
   in which methyl may be substituted by 1 to 3 fluorine substituents,
R²¹ is hydrogen or methyl,
   in which methyl may be substituted by 1 to 3 fluorine substituents,
or
R¹⁸ and R¹⁹ together with the carbon atom to which they are bonded form a 3- to 5-membered carbocycle,
or
R¹⁸ and R²⁰ together with the carbon atom to which they are bonded form a cyclopropyl ring,
with the proviso that not more than one of the R¹⁸ and R¹⁹, and R¹⁸ and R²⁰ radical pairs at the same time forms a carbocycle,
R²² is (C₁-C₆)-alkyl, cyano, 2-oxopyrrolidin-3-yl, 2-oxotetrahydrofuran-3-yl, cyclopentyl, cyclohexyl, phenyl, indanyl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
   in which (C₁-C₆)-alkyl may be substituted by a cyano radical or up to trisubstituted by fluorine,
   where phenyl may be substituted by 1 to 3 substituents selected independently from the group of fluorine, chlorine, cyano, trifluoromethyl, methyl, ethyl, methoxy and pyridyl,
   where indanyl may be substituted by hydroxyl,
   where 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl may be substituted by 1 to 3 substituents selected independently from the group of fluorine, chlorine, trifluoromethyl, (C₁-C₃)-alkyl, amino and hydroxyl,
      in which (C₁-C₃)-alkyl may be substituted by fluorine, hydroxyl, amino or trifluoromethyl,
   where cyclopentyl and cyclohexyl are substituted by cyano, methoxycarbonyl or ethoxycarbonyl,
R⁴ is hydrogen,
R⁵ is hydrogen, methyl or ethyl,
   and the N-oxides, salts, solvates, salts of the N-oxides and solvates of the N-oxides or salts thereof.

Particular preference is given in the context of the present invention to compounds of the formula (I) in which
A is CH₂,
R¹ is a phenyl group of the formula

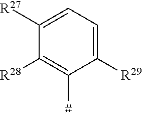

where
# is the attachment site to A,
and
R²⁷ is hydrogen or fluorine,
R²⁸ is fluorine,
R²⁹ is fluorine,
R² is methyl,
R³ is a group of the formula

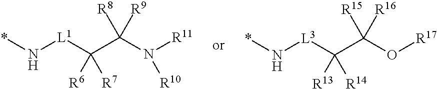

where
* is the attachment site to the carbonyl group,
L¹ is a bond,
L³ is a bond,
R⁶ is hydrogen, (C₁-C₆)-alkyl or phenyl,
   in which (C₁-C₆)-alkyl may be substituted by 1 to 5 fluorine substituents,
   and
   in which phenyl may be substituted by 1 to 2 chlorine or fluorine substituents,
R⁷ is hydrogen, methyl or ethyl,
R⁸ is hydrogen, (C₁-C₆)-alkyl, trifluoromethyl or cyclopropyl,
   in which (C₁-C₆)-alkyl may be substituted by 1 to 3 fluorine substituents,
R⁹ is hydrogen, methyl or ethyl,
R¹⁰ is hydrogen,
R¹¹ is hydrogen,
R¹³ is hydrogen, (C₁-C₆)-alkyl or phenyl,
   in which (C₁-C₆)-alkyl may be substituted by a hydroxyl radical or up to five times by fluorine,
   and
   in which phenyl may be substituted by 1 or 2 fluorine substituents,
R¹⁴ is hydrogen, methyl or ethyl,
R¹⁵ is hydrogen or (C₁-C₆)-alkyl,
R¹⁶ is hydrogen, methyl or ethyl,
or
R¹⁵ and R¹⁶ together with the carbon atom to which they are bonded form a 3- to 6-membered carbocycle,
R¹⁷ is hydrogen,
R⁴ is hydrogen,
R⁵ is hydrogen or methyl,
   and the N-oxides, salts, solvates, salts of the N-oxides and solvates of the N-oxides or salts thereof.

Particular preference is given in the context of the present invention to compounds of the formula (I) in which
A is CH₂,
R¹ is a phenyl group of the formula

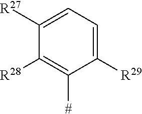

where
# is the attachment site to A,
and
R²⁷ is hydrogen or fluorine,
R²⁸ is fluorine,
R²⁹ is fluorine,
R² is methyl,
R³ is a group of the formula

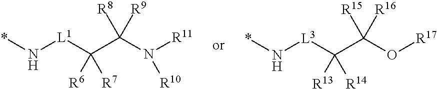

where
* is the attachment site to the carbonyl group,
L¹ is a bond,
L³ is a bond,
R⁶ is hydrogen, (C₁-C₆)-alkyl or phenyl,
   in which (C₁-C₆)-alkyl may be substituted up to five times by fluorine,
   and
   in which phenyl may be substituted by 1 to 2 chlorine or fluorine substituents,
R⁷ is hydrogen, methyl or ethyl,
R⁸ is hydrogen, (C₁-C₆)-alkyl, trifluoromethyl or cyclopropyl,
   in which (C₁-C₆)-alkyl may be up to trisubstituted by fluorine,
R⁹ is hydrogen, methyl or ethyl,
R¹⁰ is hydrogen,
R¹¹ is hydrogen,
R¹³ is hydrogen, (C₁-C₆)-alkyl or phenyl,
   in which (C₁-C₆)-alkyl may be substituted by a hydroxyl radical or up to five times by fluorine,
   and
   in which phenyl may be substituted by 1 or 2 fluorine substituents,
R¹⁴ is hydrogen, methyl or ethyl,
R¹⁵ is hydrogen or (C₁-C₆)-alkyl,
R¹⁶ is hydrogen, methyl or ethyl,
or
R¹⁵ and R¹⁶ together with the carbon atom to which they are bonded form a 3- to 6-membered carbocycle,
R¹⁷ is hydrogen,
R⁴ is hydrogen,
R⁵ is hydrogen or methyl,
   and the N-oxides, salts, solvates, salts of the N-oxides and solvates of the N-oxides or salts thereof.

In the context of the present invention, preference is also given to compounds of the formula (I) in which
R¹ is a phenyl group of the formula

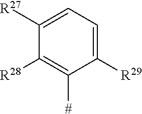

where
# is the attachment site to A,
and
R²⁷ is hydrogen or fluorine,
R²⁸ is fluorine,
R²⁹ is fluorine,
   and the N-oxides, salts, solvates, salts of the N-oxides and solvates of the N-oxides or salts thereof.

In the context of the present invention, preference is also given to compounds of the formula (I) in which
R² is methyl,
   and the N-oxides, salts, solvates, salts of the N-oxides and solvates of the N-oxides or salts thereof.

In the context of the present invention, preference is also given to compounds of the formula (I) in which
R³ is a group of the formula

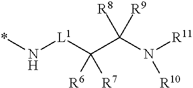

where
* is the attachment site to the carbonyl group,
L¹ is a bond,
   and the N-oxides, salts, solvates, salts of the N-oxides and solvates of the N-oxides or salts thereof.

In the context of the present invention, preference is also given to compounds of the formula (I) in which
R³ is a group of the formula

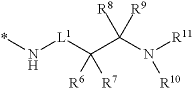

where
* is the attachment site to the carbonyl group,
R⁶ is hydrogen, (C₁-C₆)-alkyl or phenyl,
   in which (C₁-C₆)-alkyl may be substituted up to five times by fluorine,
   and
   in which phenyl may be substituted by 1 to 2 chlorine or fluorine substituents,
      and the N-oxides, salts, solvates, salts of the N-oxides and solvates of the N-oxides or salts thereof.

In the context of the present invention, preference is also given to compounds of the formula (I) in which
R³ is a group of the formula

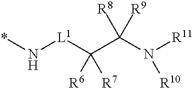

where
* is the attachment site to the carbonyl group,
R⁷ is hydrogen,
   and the N-oxides, salts, solvates, salts of the N-oxides and solvates of the N-oxides or salts thereof.

In the context of the present invention, preference is also given to compounds of the formula (I) in which
R³ is a group of the formula

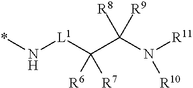

where
* is the attachment site to the carbonyl group,
and
R⁸ is hydrogen, (C₁-C₆)-alkyl, trifluoromethyl or cyclopropyl,
   in which (C₁-C₆)-alkyl may be up to trisubstituted by fluorine,
      and the N-oxides, salts, solvates, salts of the N-oxides and solvates of the N-oxides or salts thereof.

In the context of the present invention, preference is also given to compounds of the formula (I) in which
R³ is a group of the formula

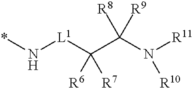

where
* is the attachment site to the carbonyl group,
and
R⁹ is hydrogen or methyl,
   and the N-oxides, salts, solvates, salts of the N-oxides and solvates of the N-oxides or salts thereof.

In the context of the present invention, preference is also given to compounds of the formula (I) in which
R³ is a group of the formula

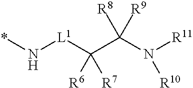

where
* is the attachment site to the carbonyl group,
and
R¹⁰ is hydrogen,
R¹¹ is hydrogen,
   and the N-oxides, salts, solvates, salts of the N-oxides and solvates of the N-oxides or salts thereof.

In the context of the present invention, preference is also given to compounds of the formula (I) in which
R³ is a group of the formula

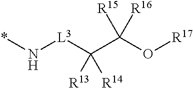

where
* is the attachment site to the carbonyl group,
L³ is a bond,
   and the N-oxides, salts, solvates, salts of the N-oxides and solvates of the N-oxides or salts thereof.

In the context of the present invention, preference is also given to compounds of the formula (I) in which
R³ is a group of the formula

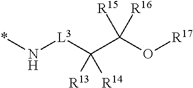

where
* is the attachment site to the carbonyl group,
R¹³ is hydrogen, (C₁-C₆)-alkyl or phenyl,
   in which (C₁-C₆)-alkyl may be substituted by a hydroxyl radical or up to five times by fluorine,
   and
   in which phenyl may be substituted by 1 or 2 fluorine substituents,
      and the N-oxides, salts, solvates, salts of the N-oxides and solvates of the N-oxides or salts thereof.

In the context of the present invention, preference is also given to compounds of the formula (I) in which
R³ is a group of the formula

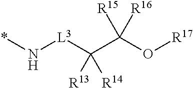

where
* is the attachment site to the carbonyl group,
R¹⁴ is hydrogen,
   and the N-oxides, salts, solvates, salts of the N-oxides and solvates of the N-oxides or salts thereof.

In the context of the present invention, preference is also given to compounds of the formula (I) in which
R³ is a group of the formula

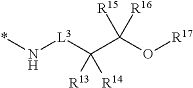

where
* is the attachment site to the carbonyl group,
and
R¹⁵ is hydrogen or (C₁-C₆)-alkyl,
   and the N-oxides, salts, solvates, salts of the N-oxides and solvates of the N-oxides or salts thereof.

In the context of the present invention, preference is also given to compounds of the formula (I) in which
R³ is a group of the formula

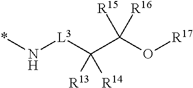

where
* is the attachment site to the carbonyl group,
and
R¹⁶ is hydrogen or methyl,
   and the N-oxides, salts, solvates, salts of the N-oxides and solvates of the N-oxides or salts thereof.

In the context of the present invention, preference is also given to compounds of the formula (I) in which
R³ is a group of the formula

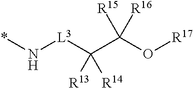

where
* is the attachment site to the carbonyl group,
and
R¹⁷ is hydrogen,
   and the N-oxides, salts, solvates, salts of the N-oxides and solvates of the N-oxides or salts thereof.

In the context of the present invention, preference is also given to compounds of the formula (I) in which
R⁵ is hydrogen or methyl,
   and the N-oxides, salts, solvates, salts of the N-oxides and solvates of the N-oxides or salts thereof.

Irrespective of the particular combinations of the radicals specified, the individual radical definitions specified in the particular combinations or preferred combinations of radicals are also replaced as desired by radical definitions from other combinations.

Particular preference is given to combinations of two or more of the preferred ranges mentioned above.

The invention further provides a process for preparing the inventive compounds of the formula (I), characterized in that

[A] a compound of the formula (II)

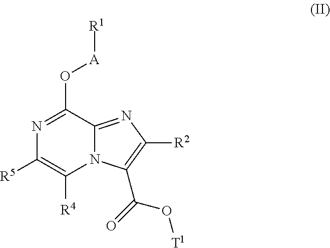

in which A, R¹, R², R⁴ and R⁵ are each as defined above and
T₁ is (C₁-C₄)-alkyl or benzyl,
is converted in an inert solvent in the presence of a suitable base or acid into a carboxylic acid of the formula (III)

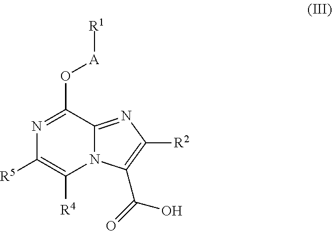

in which A, R¹, R², R⁴ and R⁵ are each as defined above,
and the latter are subsequently reacted, in an inert solvent under amide coupling conditions, with an amine of the formula (IV-A), (IV-B), (IV-C) or (IV-D)

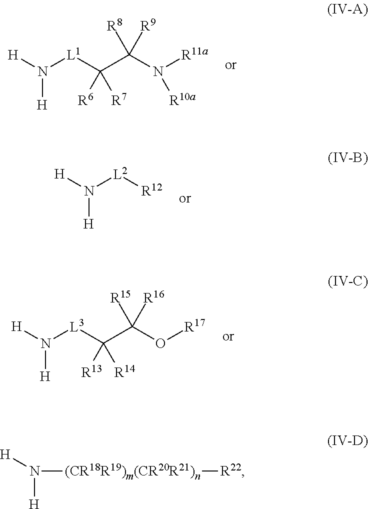

in which L¹, L², L³, R⁶, R⁷, R⁸, R⁹, R¹², R¹³, R¹⁴, R¹⁵, R¹⁶, R¹⁷, R¹⁸, R¹⁹, R²⁰, R²¹ and R²² are each as
defined above
and
R^{101A} and R^{11A} are each as defined above for R¹⁰ and R¹¹ or are an amino protecting group, for example tert-butoxycarbonyl, benzyloxycarbonyl or benzyl,
then any protecting groups present are detached, and the resulting compounds of the formula (I) are optionally converted with the appropriate (i) solvents and/or (ii) acids or bases to the solvates, salts and/or solvates of the salts thereof.

The preparation process described can be illustrated by way of example by the following synthesis scheme (Scheme 1):

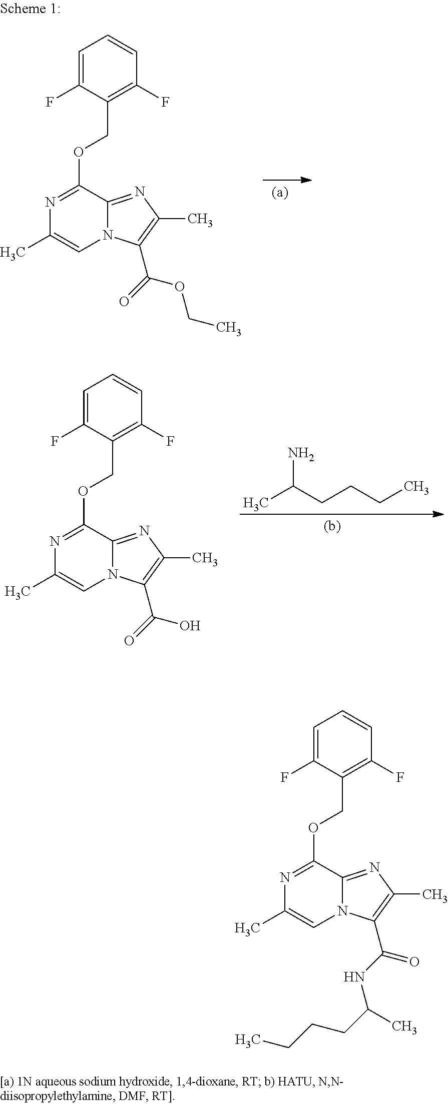

The compounds of the formulae (IV-A), (IV-B), (IV-C) and (IV-D) are commercially available or known from the literature, or can be prepared in analogy to literature processes.

Inert solvents for the process steps (III)+(IV)→(I) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halohydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N-dimethylacetamide, N,N′-dimethylpropyleneurea (DMPU) or N-methylpyrrolidone (NMP). It is likewise possible to use mixtures of the solvents mentioned. Preference is given to dichloromethane, tetrahydrofuran, dimethylformamide or mixtures of these solvents.

Suitable condensing agents for the amide formation in process steps (III)+(IV) (I) are, for example, carbodiimides such as N,N′-diethyl-, N,N′-dipropyl-, N,N′-diisopropyl- and N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline, or isobutyl chloroformate, propanephosphonic anhydride (T3P), 1-chloro-N,N,2-trimethylprop-1-en-1-amine, diethyl cyanophosphonate, bis(2-oxo-3-oxazolidinyl)phosphoryl chloride, benzotriazol-1-yloxytris (dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), 0-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu), and also as bases alkali metal carbonates, for example sodium carbonate or potassium carbonate or sodium hydrogencarbonate or potassium hydrogencarbonate, or organic bases such as trialkylamines, e.g. triethylamine, N-methylmorpholine, N-methylpiperidine or N,N-diisopropylethylamine. Preference is given to using TBTU in combination with N-methylmorpholine, HATU in combination with N,N-diisopropylethylamine or 1-chloro-N,N,2-trimethylprop-1-en-1-amine.

The condensation (III)+(IV)→(I) is generally conducted within a temperature range from −20° C. to +100° C., preferably at 0° C. to +60° C. The conversion can be carried out under atmospheric, elevated or reduced pressure (for example from 0.5 to 5 bar). In general, the reactions are carried out at atmospheric pressure.

Alternatively, the carboxylic acid of the formula (III) can also first be converted to the corresponding carbonyl chloride and the latter can then be converted directly or in a separate reaction with an amine of the formula (IV) to the compounds of the invention. The formation of carbonyl chlorides from carboxylic acids is effected by the methods known to those skilled in the art, for example by treatment with thionyl chloride, sulphuryl chloride or oxalyl chloride, in the presence of a suitable base, for example in the presence of pyridine, and optionally with addition of dimethylformamide, optionally in a suitable inert solvent.

The hydrolysis of the ester group T¹ in the compounds of the formula (II) is effected by customary methods, by treating the esters in inert solvents with acids or bases, in which latter case the salts formed at first are converted to the free carboxylic acids by treating with acid. In the case of the tert-butyl esters, the ester hydrolysis is preferably effected with acids. In the case of the benzyl esters, the ester hydrolysis is preferably effected by hydrogenolysis with palladium on activated carbon or Raney nickel. Suitable inert solvents for this reaction are water or the organic solvents customary for ester hydrolysis. These preferably include alcohols such as methanol, ethanol, n-propanol, isopropanol, n-butanol or tert-butanol, or ethers such as diethyl ether, tetrahydrofuran, 2-methyltetrahydrofuran, dioxane or glycol dimethyl ether, or other solvents such as acetone, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol.

Suitable bases for the ester hydrolysis are the customary inorganic bases. These preferably include alkali metal or alkaline earth metal hydroxides, for example sodium hydroxide, lithium hydroxide, potassium hydroxide or barium hydroxide, or alkali metal or alkaline earth metal carbonates, such as sodium carbonate, potassium carbonate or calcium carbonate. Particular preference is given to sodium hydroxide or lithium hydroxide.

Suitable acids for the ester cleavage are generally sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, optionally with addition of water. Preference is given to hydrogen chloride or trifluoroacetic acid in the case of the tert-butyl esters and to hydrochloric acid in the case of the methyl esters.

The ester hydrolysis is generally carried out within a temperature range from 0° C. to +100° C., preferably at +0° C. to +50° C.

These conversions can be performed at atmospheric, elevated or reduced pressure (for example from 0.5 to 5 bar). In general, the reactions are in each case carried out at atmospheric pressure.

The amino protecting group used is preferably tert-butylcarbonyl (Boc) or benzyloxycarbonyl (Z). Protecting groups used for a hydroxyl or carboxyl function are preferably tert-butyl or benzyl. These protecting groups are detached by customary methods, preferably by reaction with a strong acid such as hydrogen chloride, hydrogen bromide or trifluoroacetic acid in an inert solvent such as dioxane, diethyl ether, dichloromethane or acetic acid; it is optionally also possible to effect the detachment without an additional inert solvent. In the case of benzyl and benzyloxycarbonyl as protecting groups, these may also be removed by hydrogenolysis in the presence of a palladium catalyst. The detachment of the protecting groups mentioned can optionally be undertaken simultaneously in a one-pot reaction or in separate reaction steps.

The compounds of the formula (II) are known from the literature or can be prepared by reacting a compound of the formula (V)

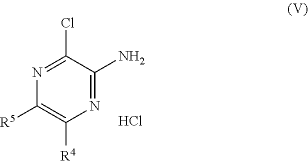

in which R⁴ and R⁵ are each as defined above
in an inert solvent in the presence of a suitable base with a compound of the formula (VI)

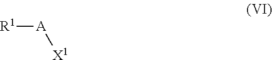

in which A and R¹ are each as defined above and
X¹ is hydroxyl
to give a compound of the formula (VII)

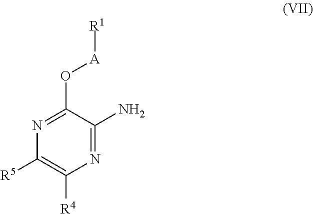

in which A, R¹, R⁴ and R⁵ are each as defined above,
and then reacting the latter in an inert solvent with a compound of the formula (VIII)

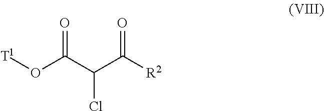

in which R² and T¹ are each as defined above.

The process described is illustrated in an exemplary manner by the scheme below (Scheme 2):

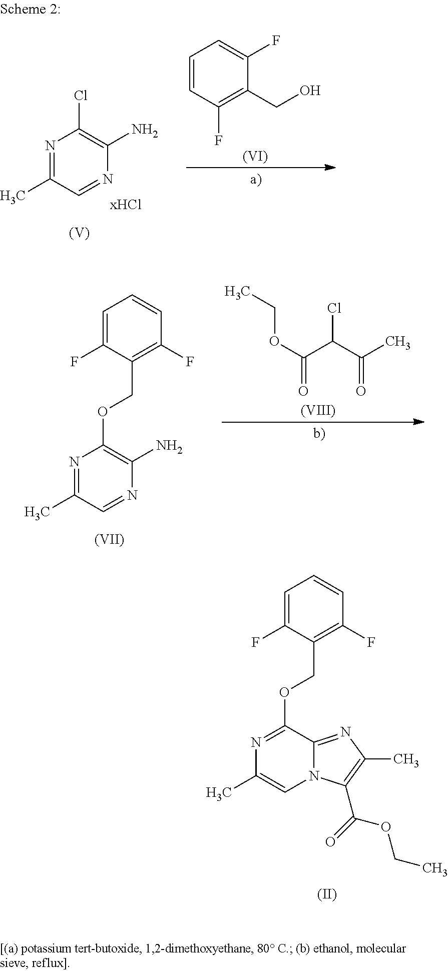

The synthesis sequence shown can be modified such that the respective reaction steps are carried out in a different order. An example of such a modified synthesis sequence is shown in Scheme 3.

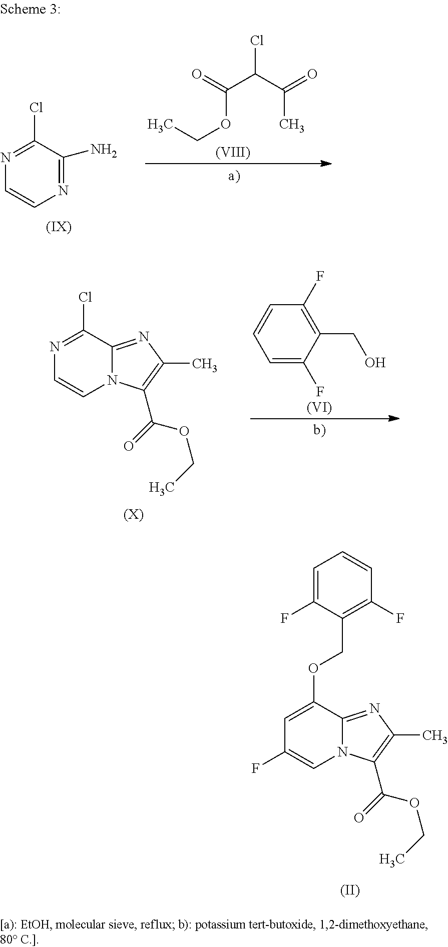

Inert solvents for the process step (V)+(VI)→(VII) or (X)+(VI)→(II) are, for example, ethers such as diethyl ether, dioxane, tetrahydrofuran, dimethoxymethane, glycol dimethyl ether or diethylene glycol dimethyl ether, or other solvents such as acetone, methyl ethyl ketone, ethyl acetate, acetonitrile, N,N-dimethylformamide, N,N-dimethylacetamide, dimethyl sulphoxide, N,N′-dimethylpropyleneurea (DMPU), N-methylpyrrolidone (NMP). It is also possible to use mixtures of the solvents mentioned. Preference is given to using dimethoxyethane.

Suitable bases for the process step (V)+(VI)→(VII) or (X)+(VI)→(II) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or caesium carbonate, optionally with addition of an alkali metal iodide, for example sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 4-(N,N-dimethylamino)pyridine (DMAP), 1,5-diazabicyclo[4.3.0]non-5-ene (DBN), 1,8-diazabicyclo[5.4.0]undec-7-ene (DBU) or 1,4-diazabicyclo[2.2.2]octane (DABCO®). Preference is given to using sodium tert-butoxide or potassium tert-butoxide.

The reaction is generally effected within a temperature range from 0° C. to +120° C., preferably at +20° C. to +80° C., optionally in a microwave. The reaction can be carried out under atmospheric, elevated or reduced pressure (for example from 0.5 to 5 bar).

Inert solvents for the ring closure to give the imidazo[1,2-a]pyrazine base skeleton (VII)+(VIII)→(II) or (VIII)+(IX)→(X) are the customary organic solvents. These preferably include alcohols such as methanol, ethanol, n-propanol, isopropanol, n-butanol, n-pentanol or tert-butanol, or ethers such as diethyl ether, tetrahydrofuran, 2-methyltetrahydrofuran, dioxane or glycol dimethyl ether, or other solvents such as acetone, dichloromethane, 1,2-dichloroethane, acetonitrile, dimethylformamide or dimethyl sulphoxide. It is also possible to use mixtures of the solvents mentioned. Preference is given to using ethanol.

The ring closure is generally effected within a temperature range from +50° C. to +150° C., preferably at +50° C. to +100° C., optionally in a microwave.

The ring closure (VII)+(VIII)→(II) or (VIII)+(IX)→(X) is optionally effected in the presence of dehydrating reaction additives, for example in the presence of molecular sieve (pore size 3A or 4A) or by means of a water separator. The reaction (VII)+(VIII)→(II) or (VIII)+(IX)→(X) is effected using an excess of the reagent of the formula (VIII), for example with 1 to 20 equivalents of the reagent (VIII), optionally with addition of bases (for example sodium hydrogencarbonate), in which case this addition can be effected all at once or in several portions.

Further compounds of the invention can optionally also be prepared by conversions of functional groups of individual substituents, especially those listed for R³, proceeding from compounds of the formula (I) obtained by above processes. These conversions are performed by customary methods known to those skilled in the art and include, for example, reactions such as nucleophilic and electrophilic substitutions, oxidations, reductions, hydrogenations, transition metal-catalysed coupling reactions, eliminations, alkylation, amination, esterification, ester cleavage, etherification, ether cleavage, formation of carbonamides, and introduction and removal of temporary protective groups.

The compounds of the invention have valuable pharmacological properties and can be used for prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for treatment and/or prophylaxis of cardiovascular, pulmonary, thromboembolic and fibrotic disorders.

The compounds of the invention can therefore be used in medicaments for treatment and/or prophylaxis of cardiovascular disorders, for example hypertension, resistant hypertension, acute and chronic heart failure, coronary heart disease, stable and unstable angina pectoris, peripheral and cardiac vascular disorders, arrhythmias, atrial and ventricular arrhythmias and impaired conduction, for example atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for treatment and/or prophylaxis of erectile dysfunction and female sexual dysfunction.

In the context of the present invention, the term “heart failure” encompasses both acute and chronic forms of heart failure, and also more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure, and acute phases of worsening of existing chronic heart failure (worsening heart failure).

In addition, the compounds of the invention can also be used for treatment and/or prophylaxis of arteriosclerosis, impaired lipid metabolism, hypolipoproteinaemias, dyslipidaemias, hypertriglyceridaemias, hyperlipidaemias, hypercholesterolaemias, abetalipoproteinaemia, sitosterolaemia, xanthomatosis, Tangier disease, adiposity, obesity and of combined hyperlipidaemias and metabolic syndrome.

The compounds of the invention can also be used for treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds of the invention are furthermore suitable for treating urological disorders, for example benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI), for example mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphatemia and/or need for dialysis. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds of the invention are also suitable for treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeld-Jacob dementia, HIV dementia, schizophrenia with dementia or Korsakoff's psychosis. They are also suitable for treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are thus effective agents for controlling migraines. They are also suitable for the prophylaxis and control of sequelae of cerebral infarction (cerebral apoplexy) such as stroke, cerebral ischaemia and craniocerebral trauma. The compounds of the invention can likewise be used for controlling states of pain and tinnitus.

In addition, the compounds of the invention have anti-inflammatory action and can therefore be used as anti-inflammatory agents for treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds of the invention can also be used for treatment and/or prophylaxis of autoimmune diseases.

The compounds of the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinized skin.

Moreover, the compounds of the invention are suitable for treatment and/or prophylaxis of hepatitis, neoplasms, osteoporosis, glaucoma and gastroparesis.

The present invention further provides for the use of the compounds according to the invention for treatment and/or prophylaxis of disorders, especially the disorders mentioned above.

The present invention further provides for the use of the compounds of the invention for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides the compounds of the invention for use in a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides for the use of the compounds of the invention for production of a medicament for treatment and/or prophylaxis of disorders, especially the aforementioned disorders.

The present invention further provides for the use of the compounds of the invention for production of a medicament for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides a method for treatment and/or prophylaxis of disorders, in particular the disorders mentioned above, using an effective amount of at least one of the compounds of the invention.

The present invention further provides a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis using an effective amount of at least one of the compounds of the invention.

The compounds of the invention can be used alone or, if required, in combination with other active ingredients. The present invention further provides medicaments comprising at least one of the compounds of the invention and one or more further active ingredients, especially for treatment and/or prophylaxis of the aforementioned disorders. Preferred examples of active ingredients suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active ingredients, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active ingredients which alter lipid metabolism, by way of example and with preference from the group of thyroid receptor agonists, cholesterol synthesis inhibitors, preferred examples being HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1-receptor blocker, by way of example and with preference prazosin.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin AII antagonist, by way of example and with preference losartan, candesartan, valsartan, telmisartan or embursatan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone or eplerenone.

In a preferred embodiment of the invention, the compounds of the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds of the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a thyroid receptor agonist such as, for example and preferably, D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorbent, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of the body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

### A. EXAMPLES

### Abbreviations and Acronyms

| | |
|---|---|
| abs. | absolute (=dried) |
| aq. | aqueous solution |
| br. | broad signal (NMR coupling pattern) |
| CAS No. | Chemical Abstracts Service number |
| δ | shift in the NMR spectrum (stated in ppm) |
| d | doublet (NMR coupling pattern) |
| TLC | thin-layer chromatography |
| DCI | direct chemical ionization (in MS) |
| DMAP | 4-N,N-dimethylaminopyridine |
| DMF | dimethylformamide |
| DMSO | dimethyl sulphoxide |
| eq. | equivalent(s) |
| ESI | electrospray ionization (in MS) |
| Et | ethyl |
| h | hour(s) |
| HATU | N-[(dimethylamino)(3H-[1,2,3]triazolo[4,5-b]-pyridin-3- |
| | yloxy)methylene]-N-methylmethanaminium |
| | hexafluorophosphate |
| HPLC | high-pressure, high-performance liquid chromatography |
| HRMS | high-resolution mass spectrometry |
| conc. | concentrated |
| LC-MS | liquid chromatography-coupled mass spectrometry |
| LiHMDS | lithium hexamethyldisilazide |
| m | multiplet (NMR coupling pattern) |
| Me | methyl |
| min | minute(s) |
| MS | mass spectrometry |
| NMR | nuclear magnetic resonance spectrometry |
| Ph | phenyl |
| q | quartet (NMR coupling pattern) |
| quint. | quintet (NMR coupling pattern) |
| R_{F} | retention factor (in thin-layer chromatography) |
| RT | room temperature |
| Rₜ | retention time (in HPLC) |
| s | singlet (NMR coupling pattern) |
| t | triplet (NMR coupling pattern) |
| THF | tetrahydrofuran |
| TBTU | (benzotriazol-1-yloxy)bisdimethylaminomethylium |
| | fluoroborate |
| UV | ultraviolet spectrometry |
| v/v | volume to volume ratio (of a solution) |

### LC-MS and HPLC Methods:

### Method 1 (LC-MS):

Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A; oven: 50° C.; flow rate: 0.33 ml/min; UV detection: 210 nm.

### Method 2 (LC-MS):

Instrument: Waters ACQUITY SQD UPLC System; column: Waters Acquity UPLC HSS T3 1.8 μ50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

### Method 3 (LC-MS):

Instrument: Micromass Quattro Premier with Waters UPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 4 (Preparative HPLC):

Column: Chromatorex C18 10μ 250×20 mm Gradient: A=water+0.5% formic acid, B=acetonitrile, 0 min=5% B, 3 min=5% B pre-rinse without substance, then injection, 5 min=5% B, 25 min=30% B, 38 min=30% B, 38.1 min=95% B, 43 min=95% B, 43.01 min=5% B, 48.0 min=5% B flow rate 20 ml/min, wavelength 210 nm.

### Method 5 (Preparative HPLC):

Column: Chromatorex C18 10μ 250×20 mm Gradient: A=water+0.5% formic acid, B=acetonitrile, 0 min=5% B, 3 min=5% B pre-rinse without substance, then injection, 5 min=5% B, 25 min=50% B, 38 min=50% B, 38.1 min=95% B, 43 min=95% B, 43.01 min=5% B, 48.0 min=5% B flow rate 20 ml/min, wavelength 210 nm.

### Method 6 (Preparative HPLC):

Column: XBridge Prep. C18 5μ 50×19 mm; gradient: A=water+0.5% ammonium hydroxide, B=acetonitrile, 0 min=5% B, 3 min=5% B pre-rinse without substance, then injection, 5 min=5% B, 25 min=50% B, 38 min=50% B, 38.1 min=95% B, 43 min=95% B, 43.01 min=5% B, 48.0 min=5% B flow rate 15 ml/min, wavelength 210 nm.

### Method 7 (MS):

Instrument: Thermo Fisher-Scientific DSQ; chemical ionization; reactant gas NH3; source temperature: 200° C.; ionization energy 70 eV.

### Method 8 (LC-MS):

Instrument: Waters ACQUITY SQD UPLC System; column: Waters Acquity UPLC HSS T3 1.8μ 30×2 mm; eluent A: 1.1 water+0.25 ml 99% formic acid, eluent B: 1.1 acetonitrile+0.25 ml 99% formic acid; gradient: 0.0 min 90% A→1.2 min 5% A→2.0 min 5% A; oven: 50° C.; flow rate: 0.60 ml/min; UV detection: 208-400 nm.

### Method 9 (Preparative HPLC):

MS instrument: Waters; HPLC instrument: Waters; Waters X-Bridge C18 column, 18 mm×50 mm, 5 μm, eluent A: water+0.05% triethylamine, eluent B: acetonitrile (ULC)+0.05% triethylamine, with gradient; flow rate: 40 ml/min; UV detection: DAD; 210-400 nm.

or:

MS instrument: Waters; HPLC instrument: Waters; Phenomenex Luna 5p C18 100A column, AXIA Tech. 50×21.2 mm, eluent A: water+0.05% formic acid, eluent B: acetonitrile (ULC)+0.05% formic acid, with gradient; flow rate: 40 ml/min; UV detection: DAD; 210-400 nm.

or:

MS instrument: Waters; HPLC instrument: Waters; Waters X-Bridge C18 column, 19 mm×50 mm, 5 μm, eluent A: water+0.05% ammonia, eluent B: acetonitrile (ULC), with gradient; flow rate: 40 ml/min; UV detection: DAD; 210-400 nm.

### Method 10 (LC-MS):

MS instrument: Waters SQD; HPLC instrument: Waters UPLC; column: Zorbax SB-Aq (Agilent), 50 mm×2.1 mm, 1.8 μm; eluent A: water+0.025% formic acid, eluent B: acetonitrile (ULC)+0.025% formic acid; gradient: 0.0 min 98% A−0.9 min 25% A−1.0 min 5% A−1.4 min 5% A−1.41 min 98% A−1.5 min 98% A; oven: 40° C.; flow rate: 0.600 ml/min; UV detection: DAD; 210 nm.

### Method 11 (MS):

Instrument: Waters ZQ 2000; electrospray ionization; eluent A: 1 l water+0.25 ml 99% formic acid, eluent B: 1 l acetonitrile+0.25 ml 99% formic acid; 25% A, 75% B; flow rate: 0.25 ml/min.

### Method 12 (GC-MS):

Instrument: Thermo Scientific DSQII, Thermo Scientific Trace GC Ultra; column: Restek RTX-35MS, 15 m×200 μm×0.33 μm; constant flow rate of helium: 1.20 ml/min; oven: 60° C.; inlet: 220° C.; gradient: 60° C., 30° C./min→300° C. (hold for 3.33 min).

### Method 13 (LC-MS):

MS instrument type: Waters Synapt G2S; UPLC instrument type: Waters Acquity I-CLASS; column: Waters, HSST3, 2.1×50 mm, C18 1.8 μm; eluent A: 1 l water+0.01% formic acid; eluent B: 1 l acetonitrile+0.01% formic acid; gradient: 0.0 min 10% B→0.3 min 10% B→1.7 min 95% B→2.5 min 95% B; oven: 50° C.; flow rate: 1.20 ml/min; UV detection: 210 nm.

### Method 14 (LC-MS):

Instrument: Waters ACQUITY SQD UPLC System; column: Waters Acquity UPLC HSS T3 1.8μ 50×1 mm; eluent A: 1 l water+0.25 ml 99% formic acid, eluent B: 1 l acetonitrile+0.25 ml 99% formic acid; gradient: 0.0 min 95% A→6.0 min 5% A→7.5 min 5% A; oven: 50° C.; flow rate: 0.35 ml/min; UV detection: 210-400 nm.

### Method 15 (LC-MS):

MS instrument: Waters (Micromass) QM; HPLC instrument: Agilent 1100 series; column: Agilent ZORBAX Extend-C18 3.0×50 mm 3.5 micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm

### Method 16 (LC-MS):

MS instrument type: Waters (Micromass) Quattro Micro; HPLC instrument type: Agilent 1100 Series; column: Thermo Hypersil GOLD 3μ 20×4 mm; eluent A: 1 l water+0.5 ml 50% formic acid, eluent B: 1 l acetonitrile+0.5 ml 50% formic acid; gradient: 0.0 min 100% A→3.0 min 10% A→4.0 min 10% A; oven: 50° C.; flow rate: 2 ml/min; UV detection: 210 nm

When compounds of the invention are purified by preparative HPLC by the above-described methods in which the eluents contain additives, for example trifluoroacetic acid, formic acid or ammonia, the compounds of the invention may be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

Salts may be present in sub- or superstoichiometric form, especially in the presence of an amine or a carboxylic acid. In addition, in the case of the present imidazopyrazines, under acidic conditions salts may always be present, even in substoichiometric amounts, without this being apparent in the ¹H NMR and without any particular specification and notification thereof in the respective IUPAC names and structural formulae.

The multiplicities of proton signals in ¹H NMR spectra reported in the paragraphs which follow represent the signal form observed in each case and do not take account of any higher-order signal phenomena.

### STARTING MATERIALS AND INTERMEDIATES

### Example 1A

### 3-[(2,6-Difluorobenzyl)oxy]-5-methylpyrazin-2-amine

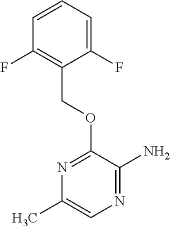 [SMILES: Cc1cnc(N)c(OCc2c(F)cccc2F)n1]

To a solution of 2.71 g of (2,6-difluorophenyl)methanol [CAS No.: 19064-18-7] (18.8 mmol, 1.3 eq.) in 120 ml of 1,2-dimethoxyethane were added 4.86 g of potassium tert-butoxide (43.3 mmol, 3.0 eq.) and the mixture was stirred at RT for 60 min. Subsequently, 2.60 g of 2-amino-3-chloro-5-methylpyrazine hydrochloride [CAS No.: 89182-14-9] (14.4 mmol, 1.0 eq.) were added and the mixture was stirred at 80° C. overnight. After cooling to room temperature, saturated aqueous sodium hydrogencarbonate solution was added and the aqueous phase was extracted three times with dichloromethane. The combined organic phases were washed with saturated aqueous sodium chloride solution, dried with magnesium sulphate, filtered and concentrated. The residue was purified by means of Biotage Isolera (340 g silica gel cartridge, cyclohexane/ethyl acetate gradient, 10%->72% ethyl acetate). 1.77 g of the title compound were obtained (39% of theory; 85% purity).

LC-MS (Method 2): Rₜ=0.94 min

MS (ESpos): m/z=252 (M+H)⁺

¹H-NMR (400 MHz, DMSO-d₆): δ [ppm]=2.20 (s, 3H), 5.35 (s, 2H), 5.88 (s, 2H), 7.09-7.23 (m, 2H), 7.37 (s, 1H), 7.46-7.57 (m, 1H).

### Example 2A

### Ethyl 8-[(2,6-difluorobenzyl)oxy]-2,6-dimethylimidazo[1,2-a]pyrazine-3-carboxylate

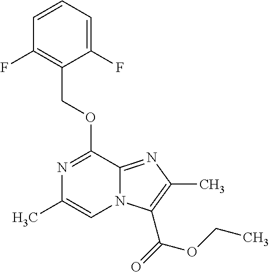 [SMILES: CCOC(=O)c1c(C)nc2c(OCc3c(F)cccc3F)nc(C)cn12]

To a solution of 1.77 g of 3-[(2,6-difluorobenzyl)oxy]-5-methylpyrazin-2-amine (7.05 mmol, 1.0 eq.) from Example 1A in 50 ml of ethanol were added 4A molecular sieve and 11.1 g of ethyl 2-chloroacetoacetate [CAS No.: 609-15-4] (70.5 mmol, 10 eq.) and the mixture was heated to reflux overnight. Subsequently, 11.1 g of ethyl 2-chloroacetoacetate (70.5 mmol, 10.0 eq) were added and the mixture was heated to reflux overnight. Then the mixture was filtered, the filtrate was concentrated, the residue obtained was extracted by stirring with diethyl ether and filtered, and the filtrate was concentrated. The residue was purified twice by means of Biotage Isolera (120 g silica gel cartridge, cyclohexane/ethyl acetate gradient). 0.81 g of the title compound was isolated (16% of theory; 52% purity).

LC-MS (Method 2): Rₜ=1.28 min

MS (ESpos): m/z=362 (M+H)⁺

### Example 3A

### 8-[(2,6-Difluorobenzyl)oxy]-2,6-dimethylimidazo[1,2-a]pyrazine-3-carboxylic acid

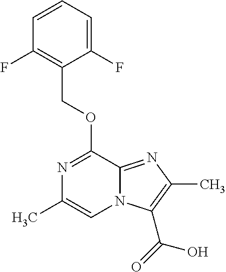 [SMILES: Cc1cn2c(C(=O)O)c(C)nc2c(OCc2c(F)cccc2F)n1]

To a solution of 800 mg of ethyl 8-[(2,6-difluorobenzyl)oxy]-2,6-dimethylimidazo[1,2-a]pyrazine-3-carboxylate (52% purity, 1.15 mmol, 1.0 eq.) from Example 2A in 10 ml of dioxane were added 5.8 ml of 1 N aqueous sodium hydroxide solution (5.8 mmol, 5 eq.) and the mixture was stirred at RT for 2 h. Subsequently, the mixture was concentrated, the residue was taken up in water and insoluble solid was filtered off. The filtrate was acidified with 1 N aqueous hydrochloric acid, and the solid formed was filtered off and dried. 354 mg of the title compound were isolated (83% of theory; 90% purity).

LC-MS (Method 2): Rₜ=0.99 min

MS (ESpos): m/z=334 (M+H)⁺

¹H-NMR (400 MHz, DMSO-d₆): δ [ppm]=2.41 (s, 3H), 2.54 (s, 3H hidden beneath solvent signal), 5.55 (s, 2H), 7.12-7.28 (m, 2H), 7.49-7.64 (m, 1H), 8.64 (s, 1H), 13.20-13.66 (br s, 1H).

### Example 4A

### rac-Benzyl (2-cyanobutan-2-yl)carbamate

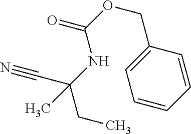 [SMILES: CCC(C)(C#N)NC(=O)OCc1ccccc1]

5.00 g (50.94 mmol) of 2-amino-2-methylbutanonitrile [synthesis described in: Lonza A G, U.S. Pat. No. 5,698,704 (1997); Deng, S. L. et al. Synthesis 2001, 2445; Hjorringgaard, C. U. et al. J. Org. Chem. 2009, 74, 1329; Ogrel, A. et al. Eur. J. Org. Chem. 2000, 857] were initially charged in 50 ml of THF and 6.5 ml of water, 21.83 g (157.92 mmol) of potassium carbonate were added and, at 0° C., 7.9 ml (56.04 mmol) of benzyl chlorocarbonate (benzyl chloroformate) were added gradually. After adding 8 ml of THF and 3 ml of water, the reaction mixture was stirred overnight, coming gradually to RT. Then water was added, and the reaction mixture was extracted three times with ethyl acetate. The combined organic phases were dried over sodium sulphate and concentrated. The residue was dissolved in diethyl ether and precipitated with petroleum ether. The product was filtered off and the solids were washed with a little petroleum ether and dried under high vacuum. 11.35 g of the target compound were obtained (93% of theory).

LC-MS (Method 2): Rₜ=0.97 min

MS (ESpos): m/z=233 (M+H)⁺

¹H-NMR (400 MHz, DMSO-d₆): δ=0.95 (t, 3H), 1.51 (s, 3H), 1.75-1.95 (m, 2H), 5.07 (s, 2H), 7.30-7.43 (m, 4H), 7.88-8.03 (m, 1H).

### Example 5A

### ent-Benzyl (2-cyanobutan-2-yl)carbamate (enantiomer A)

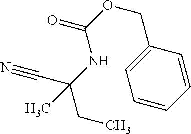 [SMILES: CCC(C)(C#N)NC(=O)OCc1ccccc1]

8 g of rac-benzyl (2-cyanobutan-2-yl)carbamate from Example 4A were separated into the enantiomers by preparative separation on a chiral phase [column: Daicel Chiralcel OJ-H, 5 μm, 250×20 mm, eluent: 50% isohexane, 50% isopropanol, flow rate: 20 ml/min; 40° C., detection: 220 nm].

Enantiomer A: yield: 3.23 g (>99% ee)

Rₜ=6.69 min [Daicel Chiralcel OJ-H, 5 μm, 250×4.6 mm; eluent: 50% isohexane, 50% isopropanol; flow rate 1.0 ml/min; 30° C.; detection: 220 nm].

### Example 6A

### ent-Benzyl (2-cyanobutan-2-yl)carbamate (enantiomer B)

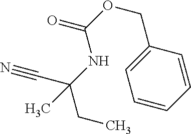 [SMILES: CCC(C)(C#N)NC(=O)OCc1ccccc1]

8 g of rac-benzyl (2-cyanobutan-2-yl)carbamate from Example 4A were separated into the enantiomers by preparative separation on a chiral phase [column: Daicel Chiralcel OJ-H, 5 μm, 250×20 mm, eluent: 50% isohexane, 50% isopropanol, flow rate: 20 ml/min; 40° C., detection: 220 nm].

Enantiomer B: yield: 3.18 g (>99% ee)

Rₜ=8.29 min [Daicel Chiralcel OJ-H, 5 μm, 250×4.6 mm; eluent: 50% isohexane, 50% isopropanol; flow rate 1.0 ml/min; 30° C.; detection: 220 nm].

### Example 7A

### ent-Benzyl (1-amino-2-methylbutan-2-yl)carbamate (enantiomer A)

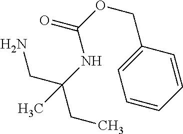 [SMILES: CCC(C)(CN)NC(=O)OCc1ccccc1]

4.00 g (17.22 mmol) of ent-benzyl (2-cyanobutan-2-yl)carbamate from Example 5A were dissolved in 50 ml of a 7 N solution of ammonia in methanol, 5.33 g of Raney nickel were added and hydrogenation was effected at about 25 bar at RT for 24 h. The mixture was filtered through Celite, washed with methanol and concentrated. The crude product was purified by means of silica gel chromatography (eluent: dichloromethane/2N ammonia in methanol=10/0.5). 2.20 g of the target compound were obtained (54% of theory).

LC-MS (Method 2): Rₜ=0.56 min

MS (ESpos): m/z=237 (M+H)⁺

### Example 8A

### ent-Benzyl (1-amino-2-methylbutan-2-yl)carbamate (enantiomer B)

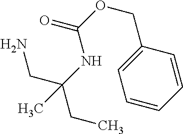 [SMILES: CCC(C)(CN)NC(=O)OCc1ccccc1]

4.00 g (17.22 mmol) of ent-benzyl (2-cyanobutan-2-yl)carbamate from Example 6A were dissolved in 50 ml of 7 N ammoniacal methanol solution, 5.33 g of Raney nickel were added and hydrogenation was effected at about 25 bar at RT for 24 h. The reaction mixture was filtered through Celite, rinsed well with methanol and concentrated. The crude product was purified by means of silica gel chromatography (eluent: dichloromethane/2N ammonia in methanol=10/0.5). 3.56 g of the target compound were obtained (87% of theory).

LC-MS (Method 3): Rₜ=1.40 min

MS (ESpos): m/z=237 (M+H)⁺

### Example 9A

### ent-Benzyl {1-[({8-[(2,6-difluorobenzyl)oxy]-2,6-dimethylimidazo[1,2-a]pyrazin-3-yl}carbonyl)amino]-2-methylbutan-2-yl}carbamate (enantiomer A)

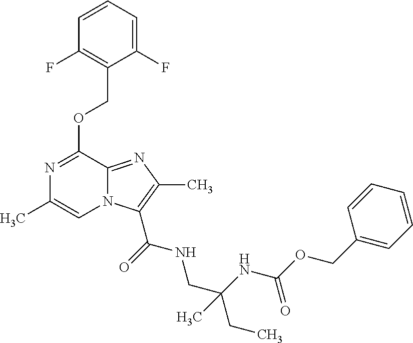 [SMILES: CCC(C)(CNC(=O)c1c(C)nc2c(OCc3c(F)cccc3F)nc(C)cn12)NC(=O)OCc1ccccc1]

To a mixture of 100 mg of 8-[(2,6-difluorobenzyl)oxy]-2,6-dimethylimidazo[1,2-a]pyrazine-3-carboxylic acid (0.30 mmol, 1.0 eq.), 106 mg of ent-benzyl (1-amino-2-methylbutan-2-yl)carbamate (enantiomer A, Example 7A) (0.450 mmol, 1.5 eq.) and 0.26 ml of N,N-diisopropylethylamine (1.50 mmol, 5.0 eq.) in 1.0 ml of DMF were added 148 mg of HATU (0.39 mmol, 1.3 eq) and the mixture was stirred at RT for 1 h. Then 70 ml of water were added and the crude product was filtered off. Subsequently, the crude product was purified by means of Biotage Isolera (10 g silica gel cartridge, cyclohexane/ethyl acetate gradient), isolating 74 mg of the title compound (41% of theory, 92% purity).

TLC (silica gel, cyclohexane/ethyl acetate 1:1): R_{F}=0.53

LC-MS (Method 2): Rₜ=1.31 min,

MS (ESpos): m/z=552 (M+H)⁺

### Example 10A

### rac-Methyl N-[(benzyloxy)carbonyl]norleucinate

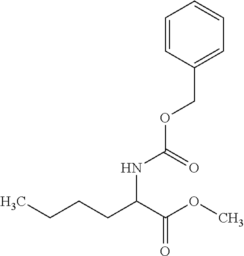 [SMILES: CCCCC(NC(=O)OCc1ccccc1)C(=O)OC]

To an initial charge of 12 g (66.05 mmol) of rac-methyl norleucinate hydrochloride in 974 ml of water/THF (8:1) were added 28.3 g (204.77 mmol) of potassium carbonate. The reaction mixture was cooled to 0° C. 12.3 ml (72.66 mmol) of benzyl chloroformate were slowly added dropwise and the reaction mixture was stirred at RT overnight. The mixture was diluted with 480 ml of water and extracted three times with dichloromethane. The combined organic phases were dried over sodium sulphate, filtered and concentrated. The residue was purified by means of silica gel chromatography (cyclohexane/ethyl acetate gradient:=4:1). This gave 18 g (97% of theory) of the target compound.

LC-MS (Method 2): Rₜ=1.10 min

MS (ESIpos): m/z=280 (M+H)⁺.

¹H-NMR (400 MHz, DMSO-d₆): δ=0.79-0.90 (m, 3H), 1.21-1.35 (m, 4H), 1.52-1.73 (m, 2H), 3.63 (s, 3H), 3.95-4.05 (m, 1H), 4.97-5.11 (m, 2H), 7.24-7.42 (m, 5H), 7.74 (d, 1H).

### Example 11A

### rac-Benzyl (2-hydroxy-2-methylheptan-3-yl)carbamate

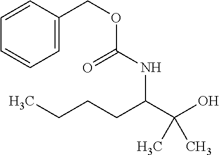 [SMILES: CCCCC(NC(=O)OCc1ccccc1)C(C)(C)O]

16.9 g (60.39 mmol) of rac-methyl N-[(benzyloxy)carbonyl]norleucinate from Example 10A were initially charged in 584 ml of THF under argon. The reaction mixture was cooled to 0° C., 70.5 ml (211.38 mmol) of 3 M methylmagnesium bromide in diethyl ether were added dropwise and the mixture was stirred at 0° C. for another 15 min. Then the mixture was allowed to warm up gradually to RT and stirred at room temperature overnight. The reaction mixture was cautiously acidified with 1 N aqueous hydrochloric acid, Celite was added to the reaction solution and the solids were filtered off. They were washed well with THF and the filtrate was concentrated. The residue was partitioned between dichloromethane and water, and the organic phase was washed twice with water, dried over sodium sulphate, filtered and concentrated. The residue was purified by means of silica gel chromatography (cyclohexane/ethyl acetate=9:1 to 7:3) and the product fractions were concentrated. This gave 15.8 g (94% of theory) of the target compound.

LC-MS (Method 2): Rₜ=0.98 min

MS (ESIpos): m/z=280 (M+H)⁺.

¹H-NMR (400 MHz, DMSO-d₆): δ=0.80-0.89 (m, 3H), 0.98 (s, 3H), 1.05 (s, 3H), 1.09-1.37 (m, 5H), 1.58-1.74 (m, 1H), 3.25-3.32 (m, 1H), 4.24 (s, 1H), 4.99-5.08 (m, 2H), 6.85 (d, 1H), 7.26-7.40 (m, 5H).

### Example 12A

### ent-Benzyl (2-hydroxy-2-methylheptan-3-yl)carbamate (enantiomer A)

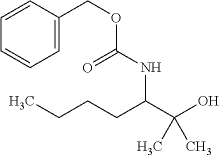 [SMILES: CCCCC(NC(=O)OCc1ccccc1)C(C)(C)O]

15.8 g of the compound from Example 11A were separated into the enantiomers by preparative separation on a chiral phase [column: Daicel Chiralpak AD-H, 5 μm, 250×20 mm, eluent: 80% isohexane, 20% ethanol, flow rate: 20 ml/min; 35° C., detection: 210 nm].

Enantiomer A:

Yield: 5.4 g (97% ee)

Rₜ=5.93 min [Daicel Chiralpak AD-H, 5 μm, 250×4.6 mm; eluent: 80% isohexane, 20% ethanol; flow rate 1.0 ml/min; 40° C.; detection: 220 nm].

### Example 13A

### ent-3-Amino-2-methylheptan-2-ol hydrochloride (enantiomer A)

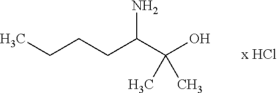

To an initial charge of 1 g (3.58 mmol) of ent-benzyl (2-hydroxy-2-methylheptan-3-yl)carbamate (enantiomer A) from Example 12A in ethanol (25 ml) under argon were added 381 mg (0.36 mmol) of 10% palladium on activated carbon and 10.9 ml (107.38 mmol) of cyclohexene, and the reaction mixture was stirred at reflux for 3 h. The mixture was filtered through a Millipore Filter® and washed through with ethanol. The filtrate was admixed with 3.6 ml (716 mmol) of 2 N aqueous hydrochloric acid in diethyl ether, then concentrated and dried under high vacuum. This gave 801 mg (123% of theory) of the target compound. The product was used in the next reaction without further purification.

DCI-MS (Method 7): m/z=146 (M−HCl+H)⁺

¹H-NMR (400 MHz, DMSO-d₆): δ=0.88 (t, 3H), 1.07 (s, 3H), 1.18 (s, 3H), 1.21-1.58 (m, 6H), 2.73-2.83 (m, 1H), 7.69-7.84 (m, 2H).

### Example 14A

### rac-Methyl 6,6,6-trifluoronorleucinate hydrochloride

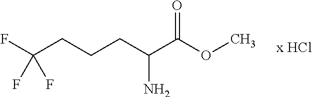

2.7 g (14.58 mmol) of rac-6,6,6-trifluoronorleucine were initially charged in 27.6 ml of saturated hydrochloric acid in methanol and stirred under reflux for 4 h. Then another 10 ml of saturated hydrochloric acid in methanol were added to the reaction solution and the mixture was stirred at reflux for a further 4 h. The reaction solution was concentrated and the residue was dried under high vacuum. 3.8 g of the target compound were obtained (99% of theory, 90% purity).

DCI-MS (Method 7): (ESpos): m/z=200 (M−HCl+H)⁺

¹H-NMR (400 MHz, DMSO-d₆): δ=1.48-1.73 (m, 2H), 1.80-1.96 (m, 2H), 2.24-2.38 (m, 2H), 3.76 (s, 3H), 4.06-4.14 (m, 1H), 8.49-8.68 (br. s, 3H).

### Example 15A

### rac-Methyl N-[(benzyloxy)carbonyl]-6,6,6-trifluoronorleucinate

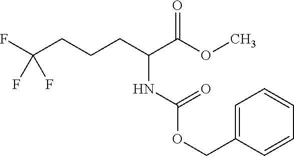 [SMILES: COC(=O)C(CCCC(F)(F)F)NC(=O)OCc1ccccc1]

To an initial charge of 3.8 g (14.39 mmol, 90% purity) of rac-methyl 6,6,6-trifluoronorleucinate hydrochloride from Example 14A in 212 ml of water/THF (8:1) were added 6.2 g (44.64 mmol) of potassium carbonate. The reaction mixture was cooled to 0° C. and 2.7 ml (15.84 mmol) of benzyl chloroformate were slowly added dropwise and then the mixture was stirred at RT overnight. The mixture was diluted with 100 ml of water and extracted three times with dichloromethane. The combined organic phases were dried over sodium sulphate, filtered and concentrated. The residue was purified by means of silica gel chromatography (cyclohexane/ethyl acetate gradient 4:1). This gave 3.6 g (76% of theory) of the target compound.

LC-MS (Method 3): Rₜ=2.32 min

MS (ESIpos): m/z=334 (M+H)⁺.

¹H-NMR (400 MHz, DMSO-d₆): δ=1.47-1.59 (m, 2H), 1.61-1.72 (m, 1H), 1.73-1.85 (m, 1H), 2.14-2.34 (m, 2H), 3.64 (s, 3H), 4.04-4.12 (m, 1H), 5.04 (s, 2H), 7.25-7.40 (m, 5H), 7.81 (d, 1H).

### Example 16A

### rac-Benzyl (7,7,7-trifluoro-2-hydroxy-2-methylheptan-3-yl)carbamate

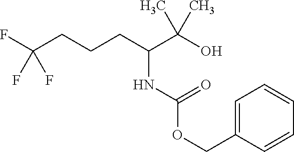 [SMILES: CC(C)(O)C(CCCC(F)(F)F)NC(=O)OCc1ccccc1]

3.2 g (9.70 mmol) of rac-methyl N-[(benzyloxy)carbonyl]-6,6,6-trifluoronorleucinate from Example 15A were initially charged in 94 ml of THF under argon. The reaction mixture was cooled to 0° C., 11.3 ml (33.96 mmol) of 3 M methylmagnesium bromide in diethyl ether were added dropwise and the mixture was stirred at 0° C. for another 15 min. The mixture was allowed to warm up gradually to RT and stirred at room temperature overnight. Saturated aqueous ammonium chloride solution was added cautiously to the reaction mixture, and then Celite. The solids were filtered off and washed well with THF, and the filtrate was concentrated. The aqueous residue was partitioned between dichloromethane and water. The organic phase was washed twice more with water, dried over sodium sulphate and filtered, and the filtrate was concentrated. The residue was purified by means of silica gel chromatography (cyclohexane/ethyl acetate 7:3) and the product fractions were concentrated. This gave 3 g (90% of theory) of the target compound.

LC-MS (Method 8): Rₜ=1.02 min

MS (ESIpos): m/z=334 (M+H)⁺.

¹H-NMR (400 MHz, DMSO-d₆): δ=0.99 (s, 3H), 1.06 (s, 3H), 1.25-1.45 (m, 2H), 1.47-1.60 (m, 1H), 1.67-1.80 (m, 1H), 2.06-2.35 (m, 2H), 3.29-3.32 (m, 1H, partly hidden by water peak), 4.32 (s, 1H), 5.05 (q, 2H), 6.95 (d, 1H), 7.26-7.38 (m, 5H).

### Example 17A

### ent-Benzyl (7,7,7-trifluoro-2-hydroxy-2-methylheptan-3-yl)carbamate (enantiomer A)

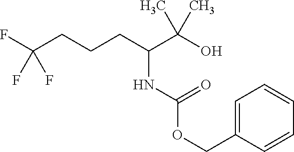 [SMILES: CC(C)(O)C(CCCC(F)(F)F)NC(=O)OCc1ccccc1]

1.9 g of the compound from Example 16A were separated into the enantiomers by preparative separation on a chiral phase [column: Daicel Chiralpak AY-H, 5 μm, 250×20 mm, eluent: 90% isohexane, 10% ethanol, flow rate: 15 ml/min; 35° C., detection: 220 nm].

Enantiomer A:

Yield: 766 mg (99% ee)

Rₜ=5.12 min [Daicel Chiralpak AY-H, 5 μm, 250×4.6 mm; eluent: 90% isohexane, 10% ethanol; flow rate 1.0 ml/min; 30° C.; detection: 220 nm].

### Example 18A

### rac-3-Amino-7,7,7-trifluoro-2-methylheptan-2-ol hydrochloride

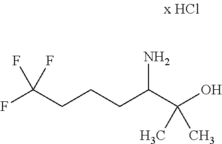

To an initial charge of 1 g (3.00 mmol) of rac-benzyl (7,7,7-trifluoro-2-hydroxy-2-methylheptan-3-yl)carbamate from Example 16A in ethanol (21 ml) under argon were added 319 mg (0.30 mmol) of 10% palladium on activated carbon and 9.1 ml (89.99 mmol) of cyclohexene, and the reaction mixture was stirred at reflux overnight. The mixture was filtered through a Millipore Filter® and washed through with ethanol. The filtrate was admixed with 3 ml (6.00 mmol) of 2 N aqueous hydrochloric acid in diethyl ether, concentrated and dried under high vacuum. This gave 785 mg (111% of theory) of the target compound. The product was used in the next reaction without further purification.

MS (Method 7): m/z=200 (M−HCl+H)⁺.

¹H-NMR (400 MHz, DMSO-d₆): δ=1.08 (s, 3H), 1.19 (s, 3H), 1.40-1.59 (m, 2H), 1.60-1.82 (m, 2H), 2.15-2.41 (m, 2H), 2.80-2.91 (m, 1H), 5.17-5.35 (br. s, 1H), 7.65-7.93 (br. s, 2H).

### Example 19A

### ent-3-Amino-7,7,7-trifluoro-2-methylheptan-2-ol hydrochloride (enantiomer A)

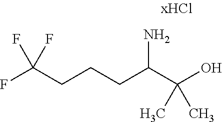

To an initial charge of 765 mg (2.29 mmol) of ent-benzyl (7,7,7-trifluoro-2-hydroxy-2-methylheptan-3-yl)carbamate (enantiomer A) from Example 17A in ethanol (16.1 ml) under argon were added 244 mg (0.23 mmol) of 10% palladium on activated carbon and 7.0 ml (68.85 mmol) of cyclohexene, and the reaction mixture was stirred at reflux for 3 h. The mixture was filtered through a Millipore® filter and washed with ethanol. The filtrate was admixed with 2.3 ml (4.59 mmol) of 2 N aqueous hydrochloric acid in diethyl ether, concentrated and dried under high vacuum. This gave 559 mg (99% of theory) of the target compound.

DCI-MS (Method 7): m/z=200 (M−HCl+H)⁺

¹H-NMR (400 MHz, DMSO-d₆): δ=1.08 (s, 3H), 1.19 (s, 3H), 1.40-1.59 (m, 2H), 1.60-1.69 (m, 1H), 1.70-1.82 (m, 1H), 2.15-2.27 (m, 1H), 2.28-2.42 (m, 1H), 2.80-2.91 (m, 1H), 5.17-5.35 (br. s, 1H), 7.73-7.97 (br. s, 2H).

### Example 20A

### 3,3,4,4,4-Pentafluorobutyl trifluoromethanesulphonate

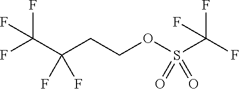 [SMILES: O=S(=O)(OCCC(F)(F)C(F)(F)F)C(F)(F)F]

198.49 g (703.51 mmol) of trifluoromethanesulphonic anhydride were initially charged under argon. The reaction flask was immersed into an oil bath at 70° C. and heated to internal temperature 56° C. 88.2 ml (738.68 mmol) of 3,3,4,4,4-pentafluorobutanol were added dropwise to the reaction mixture within 35 min and the mixture was stirred at bath temperature 70-73° C. and internal temperature 69° C. for two hours. The filtrate was concentrated on a rotary evaporator and the residue was taken up in 1500 ml of dichloromethane. The residue was washed once with 300 ml of cold water, once with 300 ml of cold saturated aqueous sodium hydrogencarbonate solution and once with 300 ml of cold water. The organic phase was dried with magnesium sulphate, filtered and concentrated. This gave 192.86 g (92.6% of theory) of the target compound.

¹H-NMR (400 MHz, DMSO-d₆): δ=2.71-2.89 (m, 2H), 4.58 (t, 2H).

### Example 21A

### rac-Methyl 5,5,6,6,6-pentafluoronorleucinate hydrochloride (racemate)

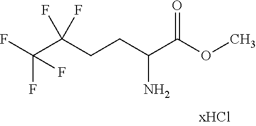

132 g (521.0 mmol) of methyl N-(diphenylmethylene)glycinate [described in: WO2010/123792 A1, 2010; p. 11-13] were initially charged in 1000 ml of THF (anhydrous) under argon and cooled to −40° C. 625.2 ml (625.20 mmol) of bis(trimethylsilyl)lithium amide (1 M in THF) were added dropwise within 30 min. After 10 min at −40° C., the internal temperature was allowed to rise to 0° C. within 35 min 192.86 g (651.25 mmol) of 3,3,4,4,4-pentafluorobutyl trifluoromethanesulphonate from Example 20A, dissolved in 400 ml of THF, were added dropwise to the reaction solution at 0° C. After 10 min, the cooling bath was removed and the mixture was stirred at RT for 3 h. Subsequently, the reaction mixture was cooled to 0° C. and 410 ml (1.33 mol) of 3 N aqueous hydrochloric acid were added dropwise. The cooling bath was removed and the reaction solution was stirred at RT for two hours. The mixture was subsequently concentrated. This gave 141.5 g of the target compound as a crude mixture (purity unknown), which was used in the subsequent stage without further purification.

### Example 22A

### rac-Methyl N-[(benzyloxy)carbonyl]-5,5,6,6,6-pentafluoronorleucinate (racemate)

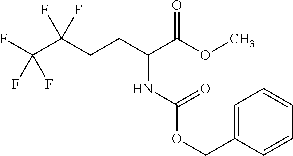 [SMILES: COC(=O)C(CCC(F)(F)C(F)(F)F)NC(=O)OCc1ccccc1]

141.5 g (520.99 mmol) of rac-methyl 5,5,6,6,6-pentafluoronorleucinate hydrochloride from Example 21A were taken up in in 850 ml of THF and 850 ml of water under argon, and 223.2 g (1.62 mol) of potassium carbonate were added cautiously at RT. Subsequently, 82 ml (573.09 mmol) of benzyl chloroformate were added dropwise and the suspension was stirred at RT overnight. The reaction mixture was extracted twice with 500 ml of ethyl acetate, and the organic phase was dried with magnesium sulphate, filtered and concentrated. The residue was diluted in 50 ml of dichloromethane and purified by means of silica gel chromatography (eluent: cyclohexane/ethyl acetate 9/1 to 4/1). The isolated product fractions were purified once more by means of preparative HPLC [column: Daiso C18 10 μm Bio 300×100 mm, neutral; eluent:acetonitrile/water gradient; flow rate: 250 ml/min; temperature: RT; wavelength: 210 nm]. This gave 27.4 g (14% of theory, 97% purity) of the target compound.

LC-MS (Method 2): Rₜ=1.09 min

MS (ESIpos): m/z=370 (M+H)⁺.

¹H-NMR (400 MHz, DMSO-d₆): δ=1.78-1.91 (m, 1H), 1.93-2.05 (m, 1H), 2.10-2.30 (m, 1H), 2.30-2.46 (m, 1H), 3.66 (s, 3H), 4.18-4.26 (m, 1H), 5.05 (s, 2H), 7.27-7.40 (m, 5H), 7.89 (d, 1H).

### Example 23A

### rac-Benzyl (6,6,7,7,7-pentafluoro-2-hydroxy-2-methylheptan-3-yl)carbamate (racemate)

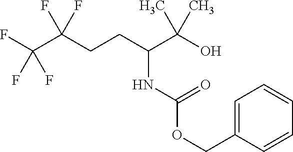 [SMILES: CC(C)(O)C(CCC(F)(F)C(F)(F)F)NC(=O)OCc1ccccc1]

1.7 g (3.68 mmol, 80% pure) of rac-methyl N-[(benzyloxy)carbonyl]-5,5,6,6,6-pentafluoronorleucinate (racemate) from Example 22A were initially charged in THF under argon and the reaction mixture was cooled to 0° C. 4.3 ml (12.89 mmol) of 3M methylmagnesium bromide in diethyl ether were added dropwise and the mixture was stirred at 0° C. for another 15 min. Then the mixture was allowed to warm up gradually to RT and stirred at room temperature overnight. Saturated aqueous ammonium chloride solution was added cautiously to the reaction mixture and then the reaction solution was concentrated to half its volume. The residue was partitioned between dichloromethane and water, and the organic phase was washed twice with water, dried over sodium sulphate, filtered and concentrated. The residue was purified by means of silica gel chromatography (cyclohexane/ethyl acetate 10:1 to 7:3). This gave 1.31 g (96% of theory) of the target compound.

LC-MS (Method 2): Rₜ=1.03 min

MS (ESIpos): m/z=370 (M+H)⁺.

¹H-NMR (400 MHz, DMSO-d₆): δ=1.01 (s, 3H), 1.08 (s, 3H), 1.43-1.56 (m, 1H), 1.92-2.01 (m, 1H), 2.01-2.19 (m, 2H), 3.36-3.44 (m, 1H), 4.48 (s, 1H), 4.99-5.12 (m, 2H), 7.11 (d, 1H), 7.27-7.38 (m, 5H).

### Example 24A

### ent-Benzyl (6,6,7,7,7-pentafluoro-2-hydroxy-2-methylheptan-3-yl)carbamate (enantiomer A)

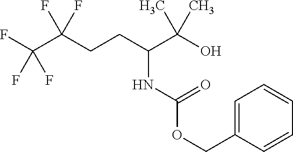 [SMILES: CC(C)(O)C(CCC(F)(F)C(F)(F)F)NC(=O)OCc1ccccc1]

1.31 g of Example 23A were separated into the enantiomers by preparative separation on a chiral phase [column: Daicel Chiralpak AY-H, 5 μm, 250×20 mm, eluent: 90% isohexane, 10% ethanol, flow rate: 15 ml/min; 35° C., detection: 220 nm].

Enantiomer A:

Yield: 459 mg (99% ee)

Rₜ=4.31 min [Daicel Chiralpak AY-H, 5 μm, 250×4.6 mm; eluent: 90% isohexane, 10% ethanol; flow rate 1.0 ml/min; 30° C.; detection: 220 nm].

### Example 25A

### ent-3-Amino-6,6,7,7,7-pentafluoro-2-methylheptan-2-ol hydrochloride (enantiomer A)

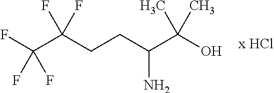

To an initial charge of 455 mg (1.23 mmol) of ent-benzyl (6,6,7,7,7-pentafluoro-2-hydroxy-2-methylheptan-3-yl)carbamate (enantiomer A) from Example 24A in 8.6 ml of ethanol were added 131 mg of palladium on charcoal (10%) and 3.74 ml (36.96 mmol) of cyclohexene, and the mixture was stirred under reflux for 3 h. The reaction mixture was filtered through a Millipore filter and washed with ethanol. The filtrate was admixed with 1.23 ml of hydrogen chloride (2 N in diethyl ether), concentrated and dried under high vacuum. 335 mg (98% of theory) of the target compound were obtained.

MS (Method 11): m/z=236 (M−HCl+H)⁺

¹H-NMR (400 MHz, DMSO-d₆): δ=1.11 (s, 3H), 1.22 (s, 3H), 1.58-1.72 (m, 1H), 1.80-1.92 (m, 1H), 2.27-2.46 (m, 2H, partly hidden by DMSO peak), 2.94-3.04 (m, 1H), 5.35 (s, 1H), 7.80-8.01 (m, 3H).

### Example 26A

### rac-2-Amino-3-fluoro-2-methylpropanonitrile

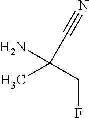

The title compound is known from the literature:
1) McConathy, J. et al., Journal of Medicinal Chemistry 2002, 45, 2240-2249.
2) Bergmann, E. D. et al., Journal of the Chemical Society 1963, 3462-3463.

### Further Method:

1.0 g (0.94 ml; 13.15 mmol) of fluoroacetone were initially charged in 11 ml of 2 N ammonia in methanol. At RT, 721 mg (14.72 mmol) of sodium cyanide and 788 mg (14.72 mmol) of ammonium chloride were added successively, and the mixture was stirred at reflux for 2 hours. The reaction solution was cooled, filtered and washed with methylene chloride. A solid precipitated out of the mother liquor, which was filtered off. Methylene chloride and methanol were distilled out of the mother liquor at standard pressure. 1.32 g of the target compound (89% of theory, about 90% purity) were obtained. The product was used in the next reaction without further purification.

GC-MS (Method 12): Rₜ=1.64 min

MS (EIpos): m/z=87 (M-CH₃)⁺

### Example 27A

### rac-Benzyl (2-cyano-1-fluoropropan-2-yl)carbamate

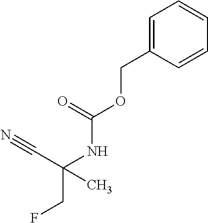 [SMILES: CC(C#N)(CF)NC(=O)OCc1ccccc1]

To 1.34 g (11.83 mmol, about 90%) of rac-2-amino-3-fluoro-2-methylpropanonitrile from Example 26A in 29 ml of THF/water (9/1) were added 5.07 g (36.67 mmol) of potassium carbonate. At 0° C., 1.69 ml (11.83 mmol) of benzyl chloroformate were slowly added dropwise and the reaction mixture was stirred at RT overnight. The solvent was decanted off and the aqueous phase was twice extracted by shaking with THF and then decanting off the THF. The combined organic phases were dried with sodium sulphate, filtered and concentrated. The residue was separated by means of silica gel chromatography (eluent: cyclohexane/ethyl acetate gradient) and the product fractions were concentrated by evaporation on a rotary evaporator. 1.89 g of the target compound were obtained (66% of theory, 97% purity).

LC-MS (Method 2): Rₜ=0.89 min

MS (ESpos): m/z=237 (M+H)⁺

¹H-NMR (400 MHz, DMSO-d₆): 6=1.58 (d, 3H), 4.47-4.78 (m, 2H), 5.10 (s, 2H), 7.30-7.43 (m, 5H), 8.34 (br. s, 1H).

### Example 28A

### ent-Benzyl (2-cyano-1-fluoropropan-2-yl)carbamate (enantiomer A)

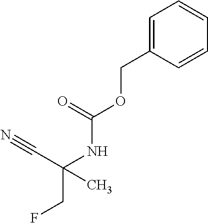 [SMILES: CC(C#N)(CF)NC(=O)OCc1ccccc1]

3.0 g (12.69 mmol) of rac-benzyl (2-cyano-1-fluoropropan-2-yl)carbamate from Example 27A were separated into the enantiomers by preparative separation on a chiral phase [column: Daicel Chiralpak AY-H, 5 μm, 250×20 mm, eluent: 80% isohexane, 20% isopropanol, flow rate: 15 ml/min; 40° C., detection: 220 nm].

Enantiomer A: Yield: 1.18 g (>99% ee)

Rₜ=5.37 min [Daicel Chiralcel AY-H, 5 μm, 250×4.6 mm; eluent: 70% isohexane, 30% 2-propanol; flow rate 1.0 ml/min; 40° C.; detection: 220 nm].

### Example 29A

### ent-Benzyl (2-cyano-1-fluoropropan-2-yl)carbamate (enantiomer B)

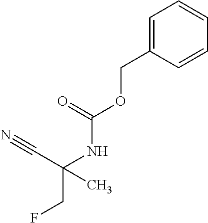 [SMILES: CC(C#N)(CF)NC(=O)OCc1ccccc1]

3.0 g (12.69 mmol) of rac-benzyl (2-cyano-1-fluoropropan-2-yl)carbamate from Example 27A were separated into the enantiomers by preparative separation on a chiral phase [column: Daicel Chiralpak AY-H, 5 μm, 250×20 mm, eluent: 80% isohexane, 20% isopropanol, flow rate: 15 ml/min; 40° C., detection: 220 nm].

Enantiomer B: Yield: 1.18 g (>99% ee)

Rₜ=6.25 min [Daicel Chiralcel AY-H, 5 μm, 250×4.6 mm; eluent: 70% isohexane, 30% 2-propanol; flow rate 1.0 ml/min; 40° C.; detection: 220 nm].

### Example 30A

### rac-Benzyl (1-amino-3-fluoro-2-methylpropan-2-yl)carbamate

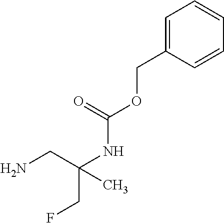 [SMILES: CC(CN)(CF)NC(=O)OCc1ccccc1]

Under argon, 1.2 g (5.08 mmol) of rac-benzyl (2-cyano-1-fluoropropan-2-yl)carbamate from Example 27A in 14.9 ml of 7 N ammonia in methanol were admixed with 1.55 g of Raney nickel (aqueous slurry) and hydrogenated at hydrogen pressure about 25 bar and RT for 24 hours. The reaction mixture was filtered through kieselguhr, washed with methanol and concentrated. 1.2 g of the target compound were obtained (98% of theory).

LC-MS (Method 2): Rₜ=0.49 min

MS (ESpos): m/z=241 (M+H)⁺

### Example 31A

### ent-Benzyl (1-amino-3-fluoro-2-methylpropan-2-yl)carbamate (enantiomer A)

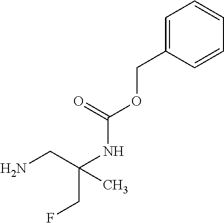 [SMILES: CC(CN)(CF)NC(=O)OCc1ccccc1]

Under argon, 1.2 g (5.08 mmol) of ent-benzyl (2-cyano-1-fluoropropan-2-yl)carbamate (enantiomer A) from Example 28A in 14.9 ml of 7 N ammonia in methanol were admixed with 1.55 g of Raney nickel (aqueous slurry) and hydrogenated at hydrogen pressure about 25 bar and RT for 24 hours. The reaction mixture was filtered through kieselguhr, washed with methanol and concentrated. 700 mg of the target compound were obtained (57% of theory, about 85% purity).

LC-MS (Method 2): Rₜ=0.52 min

MS (ESpos): m/z=241 (M+H)⁺

### Example 32A

### ent-Benzyl (1-amino-3-fluoro-2-methylpropan-2-yl)carbamate (enantiomer B)

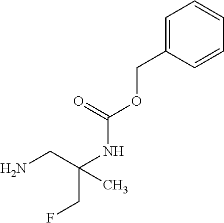 [SMILES: CC(CN)(CF)NC(=O)OCc1ccccc1]

Under argon, 1.2 g (5.08 mmol) of ent-benzyl (2-cyano-1-fluoropropan-2-yl)carbamate (enantiomer B) from Example 29A in 14.9 ml of 7 N ammonia in methanol were admixed with 1.55 g of Raney nickel (aqueous slurry) and hydrogenated at hydrogen pressure about 25 bar and RT for 24 hours. The reaction mixture was filtered through kieselguhr, washed with methanol and concentrated. 1.2 g of the target compound (98% of theory, about 85% purity) were obtained.

LC-MS (Method 2): Rₜ=0.50 min

MS (ESpos): m/z=241 (M+H)⁺

### Example 33A

### rac-2-Amino-5,5,5-trifluoro-2-methylpentanonitrile

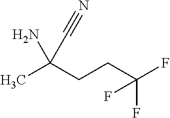

8.0 g (57.1 mmol) of 5,5,5-trifluoropentan-2-one [CAS Registry Number: 1341078-97-4; commercially available, or the methyl ketone can be prepared by literature methods which are known to those skilled in the art, for example via a) two stages from 4,4,4-trifluorobutanal according to Y. Bai et al. Angewandte Chemie 2012, 51, 4112-4116; K. Hiroi et al. Synlett 2001, 263-265; K. Mikami et al. 1982 Chemistry Letters, 1349-1352; b) or from 4,4,4-trifluorobutanoic acid according to A. A. Wube et al. Bioorganic and Medicinal Chemistry 2011, 19, 567-579; G. M. Rubottom et al. Journal of Organic Chemistry 1983, 48, 1550-1552; T. Chen et al. Journal of Organic Chemistry 1996, 61, 4716-4719. The product can be isolated by distillation or chromatography.] were initially charged in 47.8 ml of 2 N ammonia in methanol, 3.69 g (75.4 mmol) of sodium cyanide and 4.03 g (75.4 mmol) of ammonium chloride were added at room temperature and the mixture was stirred under reflux for 4 hours. The reaction mixture was cooled, diethyl ether was added and the solids present were filtered off. The solvent was distilled out of the filtrate under standard pressure. 8.7 g of the title compound (92% of theory) were obtained as residue, which was used in the subsequent stage without further purification.

GC-MS (Method 12): Rₜ=1.90 min

MS (ESpos): m/z=151 (M-CH₃)⁺

### Example 34A

### rac-Benzyl (2-cyano-5,5,5-trifluoropentan-2-yl)carbamate

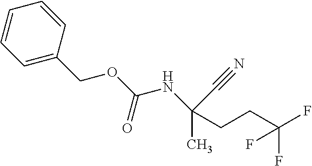 [SMILES: CC(C#N)(CCC(F)(F)F)NC(=O)OCc1ccccc1]

To an initial charge of 8.7 g (52.36 mmol) of rac-2-amino-5,5,5-trifluoro-2-methylpentanonitrile from Example 33A in 128 ml of tetrahydrofuran/water=9/1 were added 22.43 g (162.3 mmol) of potassium carbonate. At 0° C., 8.93 g (52.36 mmol) of benzyl chloroformate were slowly added dropwise. Then the mixture was allowed to warm up gradually to room temperature and stirred at room temperature overnight. The supernatant solvent was decanted off, the residue was twice stirred with 100 ml each time of tetrahydrofuran, and then the supernatant solvent was decanted off each time. The combined organic phases were concentrated and the crude product was purified by means of silica gel chromatography (eluent: cyclohexane/ethyl acetate gradient 9/1 to 4/1). 11.14 g of the title compound (68% of theory) were obtained.

LC-MS (Method 2): Rₜ=1.01 min

MS (ESpos): m/z=301 (M+H)⁺

¹H-NMR (400 MHz, DMSO-d₆): δ [ppm]=1.58 (s, 3H), 2.08-2.21 (m, 2H), 2.24-2.52 (m, 2H), 5.09 (s, 2H), 7.29-7.41 (m, 5H), 8.17 (br. s, 1H).

### Example 35A

### ent-Benzyl (2-cyano-5,5,5-trifluoropentan-2-yl)carbamate (enantiomer A)

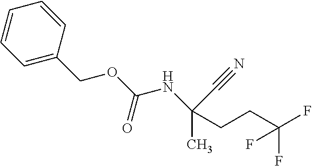 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 34A were separated into the enantiomers by preparative separation on a chiral phase [column: Daicel Chiralpak AZ-H, 5 μm, SFC, 250×50 mm, eluent: 94% carbon dioxide, 6% methanol, flow rate: 200 ml/min, temperature: 38° C., pressure: 135 bar; detection: 210 nm].

Enantiomer A: 4.12 g (about 79% ee)

Rₜ=1.60 min [SFC, Daicel Chiralpak AZ-H, 250×4.6 mm, 5 μm, eluent: 90% carbon dioxide, 10% methanol, flow rate: 3 ml/min, temperature: 30° C., detection: 220 nm].

LC-MS (Method 2): Rₜ=1.01 min

MS (ESpos): m/z=301 (M+H)⁺

### Example 36A

### ent-Benzyl (2-cyano-5,5,5-trifluoropentan-2-yl)carbamate (enantiomer B)

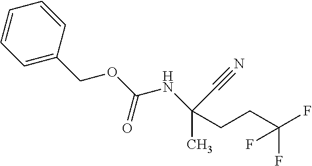 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 34A were separated into the enantiomers by preparative separation on a chiral phase [column: Daicel Chiralpak AZ-H, 5 μm, SFC, 250×50 mm, eluent: 94% carbon dioxide, 6% methanol, flow rate: 200 ml/min, temperature: 38° C., pressure: 135 bar; detection: 210 nm].

Enantiomer B: 4.54 g (about 70% ee, about 89% purity)

Rₜ=1.91 min [SFC, Daicel Chiralpak AZ-H, 250×4.6 mm, 5 μm, eluent: 90% carbon dioxide, 10% methanol, flow rate: 3 ml/min, temperature: 30° C., detection: 220 nm].

LC-MS (Method 2): Rₜ=1.01 min

MS (ESpos): m/z=301 (M+H)⁺

### Example 37A

### ent-Benzyl (1-amino-5,5,5-trifluoro-2-methylpentan-2-yl)carbamate (enantiomer A)

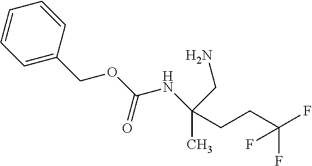 [SMILES: CC(CN)(CCC(F)(F)F)NC(=O)OCc1ccccc1]

4.12 g (13.17 mmol) of ent-benzyl (2-cyano-5,5,5-trifluoropentan-2-yl)carbamate (enantiomer A) from Example 35A were dissolved in 39 ml of 7 N ammonia solution in methanol, and 4 g of Raney nickel (50% aqueous slurry) were added under argon. The reaction mixture was hydrogenated in an autoclave at 20-30 bar overnight. Another 1 g of Raney nickel (50% aqueous slurry) was added and the reaction mixture was hydrogenated in an autoclave at 20-30 bar for 5 h. The reaction mixture was filtered through kieselguhr, rinsed with methanol and concentrated. 3.35 g (56% of theory; purity about 67%) of the target compound were obtained, which were used in the subsequent stage further without purification.

LC-MS (Method 8): Rₜ=1.68 min

MS (ESpos): m/z=305 (M+H)⁺

¹H-NMR (400 MHz, DMSO-d₆): δ [ppm]=1.13 (s, 3H), 1.40 (br. s, 2H), 1.70-1.80 (m, 1H), 1.83-1.95 (m, 1H), 2.08-2.2 (m, 2H), 4.98 (s, 2H), 6.85 (br. s, 1H), 7.28-7.41 (m, 5H).

### Example 38A

### ent-Benzyl (1-amino-5,5,5-trifluoro-2-methylpentan-2-yl)carbamate (enantiomer B)

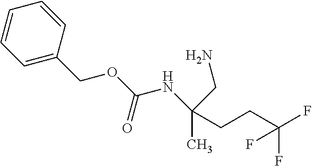 [SMILES: CC(CN)(CCC(F)(F)F)NC(=O)OCc1ccccc1]

4.54 g (13.45 mmol; purity about 89%) of ent-benzyl (2-cyano-5,5,5-trifluoropentan-2-yl)carbamate (enantiomer B) from Example 36A were dissolved in 39 ml of 7 N ammonia solution in methanol, and 5 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. 4.20 g (97% of theory; purity about 95%) of the target compound were obtained, which were used in the subsequent stage further without purification.

LC-MS (Method 15): Rₜ=2.19 min

MS (ESpos): m/z=305 (M+H)⁺

¹H-NMR (400 MHz, DMSO-d₆): δ [ppm]=1.13 (s, 3H), 1.40 (br. s, 2H), 1.69-1.80 (m, 1H), 1.83-1.96 (m, 1H), 2.07-2.22 (m, 2H), 4.98 (s, 2H), 6.85 (br. s, 1H), 7.27-7.40 (m, 5H).

### Example 39A

### rac-Benzyl (2-cyanopentan-2-yl)carbamate

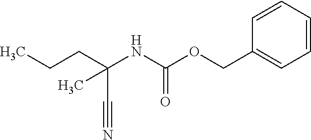 [SMILES: CCCC(C)(C#N)NC(=O)OCc1ccccc1]

20 g (178.3 mmol) of rac-2-amino-2-methylpentanonitrile (described in: Deng, S L. et al., Synthesis 2001, 2445-2449; Freifelder, M. et al., J. Am. Chem. Soc. 1960, 696-698) were initially charged in 2.63 l of THF/water (8/1), and 76.4 g (552.7 mmol) of potassium carbonate were added. Then 27.6 ml (196.1 mmol) of benzyl chloroformate were slowly added dropwise at 0° C. and the mixture was stirred at RT overnight. The reaction mixture was concentrated, and the residue was admixed with water and extracted twice with ethyl acetate. The combined organic phases were dried over sodium sulphate, filtered and concentrated. The residue was purified by means of silica gel chromatography (eluent: cyclohexane/ethyl acetate=4/1). 43.84 g of the target compound were obtained (76% of theory, 76% purity).

LC-MS (Method 2): Rₜ=1.02 min

MS (ESpos): m/z=247 (M+H)⁺

¹H-NMR (400 MHz, DMSO-d₆): δ=0.90 (t, 3H), 1.31-1.48 (m, 2H), 1.52 (s, 3H), 1.70-1.88 (m, 2H), 5.07 (s, 2H), 7.30-7.42 (m, 5H), 8.00 (br. s, 1H).

### Example 40A

### ent-Benzyl (2-cyanopentan-2-yl)carbamate (enantiomer A)

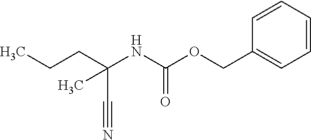 [SMILES: CCCC(C)(C#N)NC(=O)OCc1ccccc1]

43.8 g (135.3 mmol) of rac-benzyl (2-cyanopentan-2-yl)carbamate from Example 39A were separated into the enantiomers by preparative separation on a chiral phase [column: SFC Chiralpak AZ-H, 5 μm, 250×50 mm, eluent: 85% CO₂, 15% methanol, flow rate: 250 ml/min; temperature: 28° C., backpressure: 100 bar, detection: 220 nm].

Enantiomer A: Yield: 13.13 g (>99% ee)

Rₜ=2.76 min [SFC Chiralpak AZ-H, 5 μm, 250×4.6 mm; eluent: 90% CO₂, 10% methanol; flow rate: 3 ml/min; detection: 220 nm].

### Example 41A

### ent-Benzyl (2-cyanopentan-2-yl)carbamate (enantiomer B)

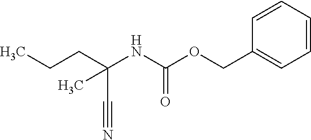 [SMILES: CCCC(C)(C#N)NC(=O)OCc1ccccc1]

43.8 g (135.3 mmol) of rac-benzyl (2-cyanopentan-2-yl)carbamate from Example 39A were separated into the enantiomers by preparative separation on a chiral phase [column: SFC Chiralpak AZ-H, 5 μm, 250×50 mm, eluent: 85% CO₂, 15% methanol, flow rate: 250 ml/min; temperature: 28° C., backpressure: 100 bar, detection: 220 nm].

Enantiomer B: Yield: 13.48 g (about 90.4% ee)

Rₜ=3.93 min [SFC Chiralpak AZ-H, 5 μm, 250×4.6 mm; eluent: 90% CO₂, 10% methanol; flow rate: 3 ml/min; detection: 220 nm].

### Example 42A

### ent-Benzyl (1-amino-2-methylpentan-2-yl)carbamate (enantiomer A)

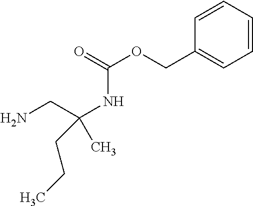 [SMILES: CCCC(C)(CN)NC(=O)OCc1ccccc1]

13.1 g (53.31 mmol) of ent-benzyl (2-cyanopentan-2-yl)carbamate (enantiomer A) from Example 40A were dissolved in 155 ml of 7 N ammonia solution in methanol, and 16.5 g of Raney nickel (50% aqueous slurry) were added under argon. The reaction mixture was hydrogenated in an autoclave at 20-30 bar overnight. The reaction mixture was filtered through Celite, rinsed with methanol, dichloromethane/2 N ammonia in methanol (20/1) and concentrated. The residue was purified by means of silica gel chromatography (eluent: dichloromethane/methanol 40/1 to 20/1). 9.85 g of the target compound were obtained (63% of theory, 86% purity).

LC-MS (Method 2): Rₜ=0.58 min

MS (ESpos): m/z=251 (M+H)⁺

¹H-NMR (400 MHz, DMSO-d₆): δ=0.83 (t, 3H), 1.11 (s, 3H), 1.15-1.24 (m, 2H), 1.37 (br. s, 2H), 1.42-1.51 (m, 1H), 1.53-1.63 (m, 1H), 2.46 (d, 1H), 2.66 (d, 1H), 4.97 (s, 2H), 6.69 (br. s, 1H), 7.26-7.40 (m, 5H).

### Example 43A

### ent-Benzyl (1-amino-2-methylpentan-2-yl)carbamate (enantiomer B)

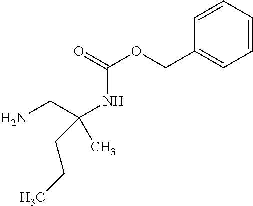 [SMILES: CCCC(C)(CN)NC(=O)OCc1ccccc1]

13.5 g (54.73 mmol) of ent-benzyl (2-cyanopentan-2-yl)carbamate (enantiomer B) from Example 41A were dissolved in 159 ml of 7 N ammonia solution in methanol, and 16.95 g of Raney nickel (50% aqueous slurry) were added under argon. The reaction mixture was hydrogenated in an autoclave at 20-30 bar overnight. The reaction mixture was filtered through Celite, rinsed with methanol, dichloromethane/2 N ammonia in methanol (10/1) and concentrated. The residue was purified by means of silica gel chromatography (eluent: dichloromethane/methanol 40/1 to 20/1). 9.46 g of the target compound were obtained (61% of theory, 88% purity).

LC-MS (Method 2): Rₜ=0.58 min

MS (ESpos): m/z=251 (M+H)⁺

¹H-NMR (400 MHz, DMSO-d₆): δ=0.83 (t, 3H), 1.11 (s, 3H), 1.15-1.24 (m, 2H), 1.37 (br. s, 2H), 1.42-1.51 (m, 1H), 1.53-1.63 (m, 1H), 2.46 (d, 1H), 2.66 (d, 1H), 4.97 (s, 2H), 6.69 (br. s., 1H), 7.26-7.40 (m, 5H).

### Example 44A

### rac-2-[(Diphenylmethylene)amino]-4-fluorobutanonitrile

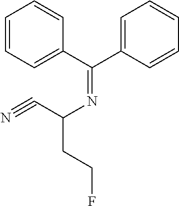 [SMILES: N#CC(CCF)N=C(c1ccccc1)c1ccccc1]

To an initial charge of 16.5 g (74.91 mmol) of [(diphenylmethylene)amino]acetonitrile in 495 ml of abs. THF were added 35.96 ml (89.89 mmol) of n-butyllithium (2.5 N in hexane) at −78° C. under argon, and the mixture was stirred at −78° C. for 15 min. Subsequently, the reaction solution was warmed up to 0° C. 13.03 g (74.91 mmol) of 1-iodo-2-fluoroethane were added dropwise to the reaction solution, which was stirred at 0° C. for a further 15 min. The reaction solution was quenched with water at 0° C., ethyl acetate was added and the mixture was washed with saturated aqueous sodium chloride solution. The aqueous phase was reextracted twice with ethyl acetate. The combined organic phases were dried over sodium sulphate, filtered and concentrated by rotary evaporation. The residue was purified by means of silica gel chromatography (eluent: dichloromethane/cyclohexane=1/1 to 2/1). 18.7 g of the target compound were obtained (80% of theory, 85% purity).

LC-MS (Method 3): Rₜ=2.42 min

MS (ESpos): m/z=267 (M+H)⁺

¹H-NMR (400 MHz, DMSO-d₆): δ=2.13-2.41 (m, 2H), 4.40 (t, 1H), 4.43-4.71 (m, 2H), 7.25-7.30 (m, 2H), 7.33-7.63 (m, 8H).

### Example 45A

### rac-2-[(Diphenylmethylene)amino]-4,4-difluorobutanonitrile

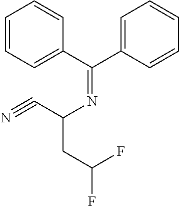 [SMILES: N#CC(CC(F)F)N=C(c1ccccc1)c1ccccc1]

To an initial charge of 18 g (81.72 mmol) of [(diphenylmethylene)amino]acetonitrile in 500 ml of abs. THF were added 39.22 ml (98.06 mmol) of n-butyllithium (2.5 N in hexane) at −78° C. under argon, and the mixture was stirred at −78° C. for a further 15 min. Subsequently, the reaction solution was warmed up to 0° C. 17.25 g (89.89 mmol) of 1,1-difluoro-2-iodoethane were added dropwise to the reaction solution, which was stirred at 0° C. for a further 15 min. The reaction solution was quenched with water at 0° C., ethyl acetate was added and the mixture was washed three times with semisaturated aqueous sodium chloride solution. The combined aqueous phases were reextracted twice with ethyl acetate. The combined organic phases were dried over sodium sulphate, filtered and concentrated by rotary evaporation. The residue was purified by means of silica gel chromatography (eluent: dichloromethane/cyclohexane 1/1). 13.57 g of the target compound were obtained (49% of theory, 84% purity).

LC-MS (Method 3): Rₜ=2.48 min

MS (ESpos): m/z=285 (M+H)⁺

¹H-NMR (400 MHz, DMSO-d₆): δ=2.53-2.61 (m, 2H; partly masked by solvent peak), 4.50 (t, 1H), 6.08-6.41 (m, 1H), 7.23-7.33 (m, 2H), 7.38-7.47 (m, 2H), 7.49-7.67 (m, 6H).

### Example 46A

### rac-2-[(Diphenylmethylene)amino]-5-fluoropentanonitrile

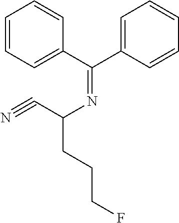 [SMILES: N#CC(CCCF)N=C(c1ccccc1)c1ccccc1]

To an initial charge of 18 g (81.72 mmol) of [(diphenylmethylene)amino]acetonitrile in 500 ml of abs. THF were added 39.22 ml (98.06 mmol) of n-butyllithium (2.5 N in hexane) at −78° C. under argon, and the mixture was stirred at −78° C. for a further 15 min. Subsequently, the reaction solution was warmed up to 0° C. and 16.9 g (89.89 mmol) of 1-fluoro-3-iodopropane were added dropwise to the reaction solution, which was stirred at 0° C. for a further 15 min. The reaction solution was quenched with water at 0° C., ethyl acetate was added and the mixture was washed with saturated aqueous sodium chloride solution. The aqueous phase was reextracted twice with ethyl acetate. The combined organic phases were dried over sodium sulphate, filtered and concentrated by rotary evaporation. The residue was purified by means of silica gel chromatography (eluent: 100% toluene, post-purification with dichloromethane/cyclohexane=1/1 to 2/1). A total of 16.73 g of the target compound (73% of theory) were obtained.

LC-MS (Method 3): Rₜ=2.50 min

MS (ESpos): m/z=281 (M+H)⁺

¹H-NMR (400 MHz, DMSO-d₆): δ=1.66-1.85 (m, 2H), 1.87-2.00 (m, 2H), 4.26-4.41 (m, 2H), 4.43-4.55 (m, 1H), 7.20-7.33 (m, 2H), 7.38-7.48 (m, 2H), 7.48-7.63 (m, 6H).

### Example 47A

### rac-2-[(Diphenylmethylene)amino]-4-fluoro-2-methylbutanonitrile

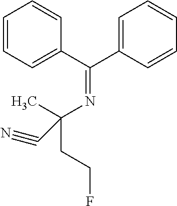 [SMILES: CC(C#N)(CCF)N=C(c1ccccc1)c1ccccc1]

To an initial charge of 19.94 g (63.64 mmol, 85% purity) of rac-2-[(diphenylmethylene)amino]-4-fluorobutanonitrile from Example 44A in 421 ml of abs. THF were added 25.71 ml (64.28 mmol) of n-butyllithium (2.5 N in hexane) at −78° C. under argon, and the mixture was stirred at −78° C. for a further 10 min. Subsequently, 36.1 g (254.57 mmol) of iodomethane were added to the reaction solution at −78° C. The reaction mixture was gradually brought to 0° C. over 4.5 h. After complete depletion of the starting material, the reaction solution was quenched with water at 0° C., ethyl acetate was added and the mixture was washed twice with saturated aqueous sodium chloride solution. The organic phase was dried over sodium sulphate, filtered and concentrated by rotary evaporation. The residue was purified by means of silica gel chromatography (eluent: cyclohexane/ethyl acetate=15/1). 17.2 g of the target compound were obtained (78% of theory, 81% purity).

LC-MS (Method 3): Rₜ=2.46 min

MS (ESpos): m/z=281 (M+H)⁺

¹H-NMR (400 MHz, DMSO-d₆): δ=1.65-1.67 (s, 3H), 2.30-2.47 (m, 2H), 4.55-4.84 (m, 2H), 7.27-7.32 (m, 2H), 7.37-7.42 (m, 2H), 7.43-7.52 (m, 6H).

### Example 48A

### rac-2-[(Diphenylmethylene)amino]-4,4-difluoro-2-methylbutanonitrile

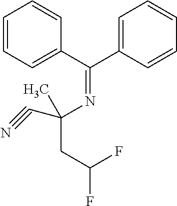 [SMILES: CC(C#N)(CC(F)F)N=C(c1ccccc1)c1ccccc1]

To an initial charge of 13.07 g (38.62 mmol) of rac-2-[(diphenylmethylene)amino]-4,4-difluorobutanonitrile from Example 45A in 255 ml of abs. THF were added 15.6 ml (39.0 mmol) of n-butyllithium (2.5 N in hexane) at −78° C. under argon, and the mixture was stirred at −78° C. for a further 10 min. Subsequently, 22.6 g (154.46 mmol) of iodomethane were added to the reaction solution at −78° C. The reaction mixture was gradually brought to 0° C. over 3.5 h. After complete depletion of the starting material, the reaction solution was quenched with water at 0° C., ethyl acetate was added and the mixture was washed twice with saturated aqueous sodium chloride solution. The organic phase was dried over sodium sulphate, filtered and concentrated by rotary evaporation. The residue was purified by means of silica gel chromatography (eluent: cyclohexane/ethyl acetate=15/1). 11.4 g of the target compound were obtained (91% of theory, 92% purity).

LC-MS (Method 3): Rₜ=2.52 min

MS (ESpos): m/z=299 (M+H)⁺

¹H-NMR (400 MHz, DMSO-d₆): δ=1.67 (s, 3H), 2.55-2.77 (m, 2H), 6.14-6.48 (m, 1H), 7.28-7.34 (m, 2H), 7.36-7.44 (m, 2H), 7.44-7.54 (m, 6H).

### Example 49A

### rac-2-[(Diphenylmethylene)amino]-5-fluoro-2-methylpentanonitrile

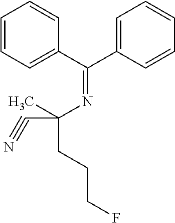 [SMILES: CC(C#N)(CCCF)N=C(c1ccccc1)c1ccccc1]

To an initial charge of 16.73 g (59.68 mmol) of rac-2-[(diphenylmethylene)amino]-5-fluoropentanonitrile from Example 46A in 394 ml of abs. THF were added 24.11 ml (60.27 mmol) of n-butyllithium (2.5 N in hexane) at −78° C. under argon, and the mixture was stirred at −78° C. for a further 10 min. Subsequently, 34.93 g (238.70 mmol) of iodomethane were added to the reaction solution at −78° C. The reaction mixture was gradually brought to 0° C. over 4.5 h. The reaction solution was quenched with water at 0° C., ethyl acetate was added and the mixture was washed twice with saturated aqueous sodium chloride solution. The organic phase was dried over sodium sulphate, filtered and concentrated by rotary evaporation. The residue was purified by means of silica gel chromatography (eluent: cyclohexane/ethyl acetate=15/1). 18.94 g of the target compound were obtained (95% of theory, 88% purity).

LC-MS (Method 3): Rₜ=2.55 min

MS (ESpos): m/z=295 (M+H)⁺

¹H-NMR (400 MHz, DMSO-d₆): δ=1.62 (s, 3H), 1.73-1.90 (m, 2H), 1.94-2.03 (m, 1H), 2.04-2.18 (m, 1H), 4.47 (t, 1H), 4.58 (t, 1H), 7.23-7.33 (m, 2H), 7.35-7.43 (m, 2H), 7.44-7.56 (m, 6H).

### Example 50A

### rac-2-Amino-4-fluoro-2-methylbutanonitrile hydrochloride

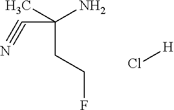

17.45 g (50.45 mmol; 81% purity) of rac-2-[(diphenylmethylene)amino]-4-fluoro-2-methylbutanonitrile from Example 47A were dissolved in 235.6 ml of tetrahydrofuran and 9.1 ml of water, 111 ml (55.46 mmol) of hydrogen chloride solution (0.5 N in diethyl ether) were added and the mixture was stirred at room temperature overnight. The slightly turbid reaction solution was admixed with 25.21 ml (50.42 mmol) of hydrogen chloride solution (2 N in diethyl ether) and concentrated by rotary evaporation. The isolated crude product was reacted further directly without further purification.

LC-MS (Method 3): Rₜ=0.22 min

MS (ESpos): m/z=117 (M−HCl+H)⁺

### Example 51A

### rac-Benzyl (2-cyano-4-fluorobutan-2-yl)carbamate

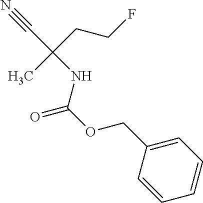 [SMILES: CC(C#N)(CCF)NC(=O)OCc1ccccc1]

To an initial charge of the crude rac-2-amino-4-fluoro-2-methylbutanonitrile hydrochloride product from Example 50A in 165 ml of tetrahydrofuran/water (1:1) were added 28.57 g (206.71 mmol) of potassium carbonate and 9.46 g (55.46 mmol) of benzyl chloroformate. The reaction mixture (biphasic mixture) was stirred at room temperature overnight. Another 1.72 g (10.1 mmol) of benzyl chloroformate were added to the reaction and the mixture was stirred at room temperature for a further 2 h. Subsequently, the biphasic system was separated and the aqueous phase was extracted twice with ethyl acetate. The combined organic phases were washed once with saturated aqueous sodium chloride solution, and then dried over sodium sulphate, filtered and concentrated by rotary evaporation. The residue was purified by means of silica gel chromatography (eluent: cyclohexane/ethyl acetate gradient=20/1 to 5/1). 5.04 g of the target compound were obtained (38% of theory over two stages, 96% purity).

LC-MS (Method 3): Rₜ=1.95 min

MS (ESpos): m/z=251 (M+H)⁺

¹H-NMR (400 MHz, DMSO-d₆): δ [ppm]=1.59 (s, 3H), 2.20-2.43 (m, 2H), 4.55 (t, 1H), 4.67 (t, 1H), 5.08 (s, 2H), 7.28-7.45 (m, 5H), 8.12 (br. s, 1H).

### Example 52A

### rac-2-Amino-4,4-difluoro-2-methylbutanonitrile hydrochloride

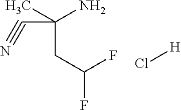

10.84 g (33.43 mmol; 92% purity) of rac-2-[(diphenylmethylene)amino]-4,4-difluoro-2-methylbutanonitrile from Example 48A were dissolved in 156 ml of tetrahydrofuran and 6 ml of water, 73.5 ml (36.77 mmol) of hydrogen chloride solution (0.5 N in diethyl ether) were added and the mixture was stirred at room temperature overnight. The reaction solution was admixed with 16.71 ml (33.43 mmol) of hydrogen chloride solution (2 N in diethyl ether) and concentrated by rotary evaporation. The isolated crude product was reacted further directly without further purification.

LC-MS (Method 3): Rₜ=0.32 min

MS (ESpos): m/z=135 (M−HCl+H)⁺

### Example 53A

### rac-Benzyl (2-cyano-4,4-difluorobutan-2-yl)carbamate

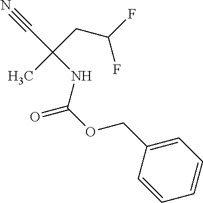 [SMILES: CC(C#N)(CC(F)F)NC(=O)OCc1ccccc1]

To an initial charge of the crude rac-2-amino-4,4-difluoro-2-methylbutanonitrile hydrochloride product from Example 52A in 109 ml of tetrahydrofuran/water (1:1) were added 18.94 g (137.06 mmol) of potassium carbonate and 6.27 g (36.77 mmol) of benzyl chloroformate. The reaction mixture (biphasic mixture) was stirred at room temperature overnight. Another 1.14 g (6.69 mmol) of benzyl chloroformate were added to the reaction and the mixture was stirred at room temperature for a further 2 h. Subsequently, the biphasic system was separated and the aqueous phase was extracted twice with ethyl acetate. The combined organic phases were washed once with saturated aqueous sodium chloride solution, and then dried over sodium sulphate, filtered and concentrated by rotary evaporation. The residue was purified by means of silica gel chromatography (eluent: cyclohexane/ethyl acetate gradient=20/1 to 5/1). 7.68 g of the target compound were obtained (61% of theory over two stages, 71% purity).

LC-MS (Method 3): Rₜ=2.04 min

MS (ESpos): m/z=269 (M+H)⁺

¹H-NMR (400 MHz, DMSO-d₆): δ [ppm]=1.65 (s, 3H), 2.51-2.65 (m, 2H), 5.10 (s, 2H), 6.08-6.41 (m, 1H), 7.27-7.44 (m, 5H), 8.24 (br. s, 1H).

### Example 54A

### rac-2-Amino-5-fluoro-2-methylpentanonitrile hydrochloride

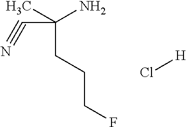

18.94 g (56.62 mmol; 88% purity) of rac-2-[(diphenylmethylene)amino]-5-fluoro-2-methylpentanonitrile from Example 49A were dissolved in 264.6 ml of tetrahydrofuran and 10.2 ml of water, 124.6 ml (62.28 mmol) of hydrogen chloride solution (0.5 N in diethyl ether) were added and the mixture was stirred at room temperature overnight. The reaction solution was admixed with 28.3 ml (56.62 mmol) of hydrogen chloride solution (2 N in diethyl ether) and concentrated by rotary evaporation. The isolated crude product was reacted further directly without further purification.

LC-MS (Method 3): Rₜ=0.25 min

MS (ESpos): m/z=131 (M−HCl+H)⁺

### Example 55A

### rac-Benzyl (2-cyano-5-fluoropentan-2-yl)carbamate

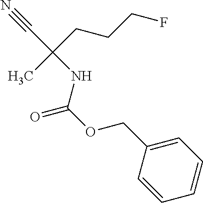 [SMILES: CC(C#N)(CCCF)NC(=O)OCc1ccccc1]

To an initial charge of the crude rac-2-amino-5-fluoro-2-methylpentanonitrile hydrochloride product from Example 54A in 185 ml of tetrahydrofuran/water (1/1) were added 32.09 g (232.18 mmol) of potassium carbonate and 10.63 g (62.29 mmol) of benzyl chloroformate. The reaction mixture (biphasic mixture) was stirred at room temperature overnight. Another 1.93 g (11.33 mmol) of benzyl chloroformate were added to the reaction and the mixture was stirred at room temperature for a further 2 h. Subsequently, the biphasic system was separated and the aqueous phase was extracted twice with ethyl acetate. The combined organic phases were washed once with saturated aqueous sodium chloride solution, and then dried over sodium sulphate, filtered and concentrated by rotary evaporation. The residue was purified by means of silica gel chromatography (eluent: cyclohexane/ethyl acetate gradient=20/1 to 5/1). 11.77 g of the target compound were obtained (72% of theory over two stages, 92% purity).

LC-MS (Method 3): Rₜ=2.03 min

MS (ESpos): m/z=265 (M+H)⁺

¹H-NMR (400 MHz, DMSO-d₆): δ [ppm]=1.55 (s, 3H), 1.66-1.85 (m, 2H), 1.86-2.04 (m, 2H), 4.40 (t, 1H), 4.52 (t, 1H), 5.08 (s, 2H), 7.28-7.44 (m, 5H), 8.05 (br. s, 1H).

### Example 56A

### ent-Benzyl (2-cyano-4,4-difluorobutan-2-yl)carbamate (enantiomer A)

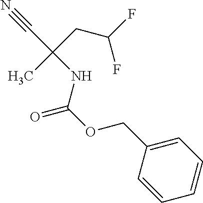 [SMILES: CC(C#N)(CC(F)F)NC(=O)OCc1ccccc1]

7.68 g (20.33 mmol, 71% purity) of rac-benzyl (2-cyano-4,4-difluorobutan-2-yl)carbamate from Example 53A were separated into the enantiomers by preparative separation on a chiral phase [column: Daicel Chiralpak AY-H, 5 μm, 250×20 mm, eluent: 80% isohexane, 20% isopropanol, flow rate: 25 ml/min; temperature: 22° C., detection: 210 nm].

Enantiomer A: Yield: 2.64 g (>99% ee)

Rₜ=6.67 min [Chiralpak AY-H, 5 μm, 250×4.6 mm; eluent: 80% isohexane, 20% isopropanol; flow rate: 3 ml/min; detection: 220 nm].

### Example 57A

### ent-Benzyl (2-cyano-4,4-difluorobutan-2-yl)carbamate (enantiomer B)

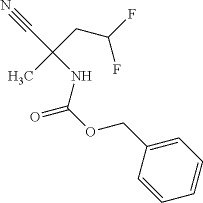 [SMILES: CC(C#N)(CC(F)F)NC(=O)OCc1ccccc1]

7.68 g (20.33 mmol, 71% purity) of rac-benzyl (2-cyano-4,4-difluorobutan-2-yl)carbamate from Example 53A were separated into the enantiomers by preparative separation on a chiral phase [column: Daicel Chiralpak AY-H, 5 μm, 250×20 mm, eluent: 80% isohexane, 20% isopropanol, flow rate: 25 ml/min; temperature: 22° C., detection: 210 nm].

Enantiomer B: Yield: 2.76 g (93% ee)

Rₜ=7.66 min [Chiralpak AY-H, 5 μm, 250×4.6 mm; eluent: 80% isohexane, 20% isopropanol; flow rate: 3 ml/min; detection: 220 nm].

### Example 58A

### ent-Benzyl (2-cyano-5-fluoropentan-2-yl)carbamate (enantiomer A)

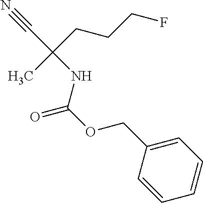 [SMILES: CC(C#N)(CCCF)NC(=O)OCc1ccccc1]

11.77 g (40.97 mmol, 92% purity) of rac-benzyl (2-cyano-5-fluoropentan-2-yl)carbamate from Example 55A were separated into the enantiomers by preparative separation on a chiral phase [column: SFC Daicel Chiralpak AZ-H, 5 μm, 250×30 mm, eluent: 90% CO₂, 10% methanol, flow rate: 100 ml/min; temperature: 40° C., detection: 210 nm].

Enantiomer A: Yield: 5.7 g (>99% ee)

Rₜ=1.76 min [SFC Chiralpak AZ-3, 3 μm, 50×4.6 mm; eluent: CO₂/methanol gradient (5% to 60% methanol); flow rate: 3 ml/min; detection: 220 nm].

### Example 59A

### ent-Benzyl (2-cyano-5-fluoropentan-2-yl)carbamate (enantiomer B)

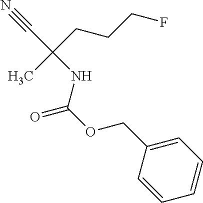 [SMILES: CC(C#N)(CCCF)NC(=O)OCc1ccccc1]

11.77 g (40.97 mmol, 92% purity) of rac-benzyl (2-cyano-5-fluoropentan-2-yl)carbamate from Example 55A were separated into the enantiomers by preparative separation on a chiral phase [column: SFC Daicel Chiralpak AZ-H, 5 μm, 250×30 mm, eluent: 90% CO₂, 10% methanol, flow rate: 100 ml/min; temperature: 40° C., detection: 210 nm].

Enantiomer B: Yield: 5.0 g (>99% ee)

Rₜ=1.97 min [SFC Chiralpak AZ-3, 3 μm, 50×4.6 mm; eluent: CO₂/methanol gradient (5% to 60% methanol); flow rate: 3 ml/min; detection: 220 nm].

### Example 60A

### ent-Benzyl (1-amino-4,4-difluoro-2-methylbutan-2-yl)carbamate (enantiomer A)

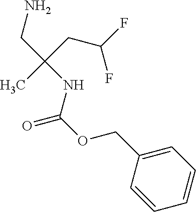 [SMILES: CC(CN)(CC(F)F)NC(=O)OCc1ccccc1]

2.3 g (8.57 mmol) of ent-benzyl (2-cyano-4,4-difluorobutan-2-yl)carbamate (enantiomer A) from Example 56A were dissolved in 75 ml of 7 N ammonia solution in methanol, and 2.66 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. 2.23 g of the target compound were obtained (94% of theory, 98% purity).

LC-MS (Method 3): Rₜ=1.48 min

MS (ESpos): m/z=273 (M+H)+

¹H-NMR (400 MHz, DMSO-d₆): δ [ppm]=1.19 (s, 3H), 1.48 (br. s, 2H), 2.08-2.40 (m, 2H), 2.53-2.72 (m, 2H; partly masked by solvent peak), 5.00 (s, 2H), 5.90-6.23 (m, 1H), 6.95 (br. s, 1H), 7.25-7.41 (m, 5H).

### Example 61A

### ent-Benzyl (1-amino-4,4-difluoro-2-methylbutan-2-yl)carbamate (enantiomer B)

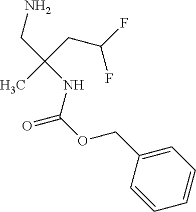 [SMILES: CC(CN)(CC(F)F)NC(=O)OCc1ccccc1]

2.76 g (10.29 mmol) of ent-benzyl (2-cyano-4,4-difluorobutan-2-yl)carbamate (enantiomer B) from Example 57A were dissolved in 90 ml of 7 N ammonia solution in methanol, and 3.19 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. 2.64 g of the target compound were obtained (88% of theory, 93% purity).

LC-MS (Method 3): Rₜ=1.49 min

MS (ESpos): m/z=273 (M+H)⁺

¹H-NMR (400 MHz, DMSO-d₆): δ [ppm]=1.19 (s, 3H), 1.48 (br. s, 2H), 2.08-2.40 (m, 2H), 2.53-2.73 (m, 2H; partly masked by solvent peak), 5.00 (s, 2H), 5.90-6.24 (m, 1H), 6.95 (br. s, 1H), 7.25-7.41 (m, 5H).

### Example 62A

### ent-Benzyl (1-amino-5-fluoro-2-methylpentan-2-yl)carbamate (enantiomer A)

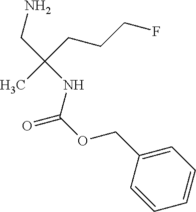 [SMILES: CC(CN)(CCCF)NC(=O)OCc1ccccc1]

5.7 g (21.57 mmol) of ent-benzyl (2-cyano-5-fluoropentan-2-yl)carbamate (enantiomer A) from Example 58A were dissolved in 125 ml of 7 N ammonia solution in methanol, and 6.68 g of Raney nickel (50% aqueous slurry) were added under argon. The reaction mixture was hydrogenated in an autoclave at 20-30 bar for 4.5 h. The reaction mixture was filtered through Celite, rinsed with methanol and 2 N ammonia in methanol, and concentrated. 5.22 g of the target compound were obtained (77% of theory, 85% purity).

LC-MS (Method 3): Rₜ=1.51 min

MS (ESpos): m/z=269 (M+H)⁺

### Example 63A

### ent-Benzyl (1-amino-5-fluoro-2-methylpentan-2-yl)carbamate (enantiomer B)

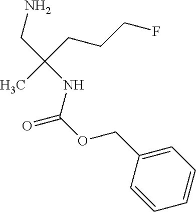 [SMILES: CC(CN)(CCCF)NC(=O)OCc1ccccc1]

5.0 g (18.92 mmol) of ent-benzyl (2-cyano-5-fluoropentan-2-yl)carbamate (enantiomer B) from Example 59A were dissolved in 110 ml of 7 N ammonia solution in methanol, and 5.86 g of Raney nickel (50% aqueous slurry) were added under argon. The reaction mixture was hydrogenated in an autoclave at 20-30 bar for 4.5 h. The reaction mixture was filtered through Celite, rinsed with methanol and 2 N ammonia in methanol, and concentrated. 4.6 g of the target compound were obtained (84% of theory, 93% purity).

LC-MS (Method 3): Rₜ=1.47 min

MS (ESpos): m/z=269 (M+H)⁺

### Example 64A

### rac-4-Fluoro-2-methylbutane-1,2-diamine dihydrochloride

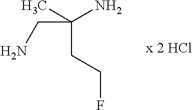

1.00 g (4.00 mmol) of rac-benzyl (2-cyano-4-fluorobutan-2-yl)carbamate from Example 51A were dissolved in 114 ml ethanol/glacial acetic acid (1/1), and 0.85 g of palladium on activated carbon (10%) were added. The reaction mixture was hydrogenated in an autoclave at 30-50 bar for 3 h. The reaction mixture was filtered through a fluted filter, rinsed with ethanol and then filtered once again through a Millipore filter. The filtrate was admixed with 10 ml of hydrogen chloride solution (2 N in diethyl ether) and then concentrated by evaporation. 1.04 g of the target compound were obtained, which was used in the subsequent stage without further purification.

LC-MS (Method 3): Rₜ=0.19 min

MS (ESpos): m/z=121 (M-2HCl+H)⁺

### Example 65A

### ent-Benzyl {1-[({8-[(2,6-difluorobenzyl)oxy]-2,6-dimethylimidazo[1,2-a]pyrazin-3-yl}carbonyl)amino]-2-methylpentan-2-yl}carbamate trifluoroacetate (enantiomer B)

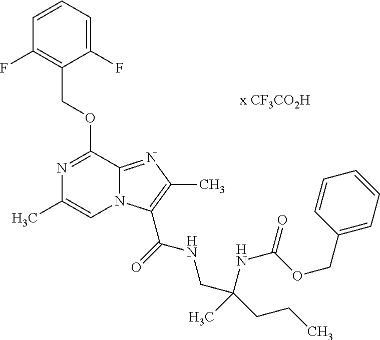 [SMILES: CCCC(C)(CNC(=O)c1c(C)nc2c(OCc3c(F)cccc3F)nc(C)cn12)NC(=O)OCc1ccccc1]

An initial charge of 50 mg (0.15 mmol) of 8-[(2,6-difluorobenzyl)oxy]-2,6-dimethylimidazo[1,2-a]pyrazine-3-carboxylic acid from Example 3A together with 74 mg (0.20 mmol) of HATU and 0.13 ml (0.75 mmol) of N,N-diisopropylethylamine in 0.5 ml of DMF was stirred at room temperature for 20 min. Subsequently, 49 mg (0.17 mmol; 88% purity) of ent-benzyl (1-amino-2-methylpentan-2-yl)carbamate from Example 43A (enantiomer B) were added to the reaction solution and the mixture was stirred at RT overnight. Then the mixture was diluted with acetonitrile and water, admixed with TFA and purified by means of preparative HPLC (RP18 column, eluent: acetonitrile/water gradient with addition of 0.1% TFA). The product fractions were combined, concentrated and lyophilized. 66 mg of the target compound (65% of theory) were obtained.

LC-MS (Method 2): Rₜ=1.31 min

MS (ESpos): m/z=566 (M-TFA+H)⁺

### Example 66A

### ent-Benzyl {1-[({8-[(2,6-difluorobenzyl)oxy]-2,6-dimethylimidazo[1,2-a]pyrazine-3-yl}carbonyl)amino]-3-fluoro-2-methylpropan-2-yl}carbamate (enantiomer B)

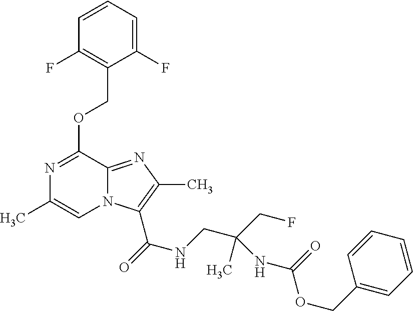 [SMILES: Cc1cn2c(C(=O)NCC(C)(CF)NC(=O)OCc3ccccc3)c(C)nc2c(OCc2c(F)cccc2F)n1]

An initial charge of 60 mg (0.18 mmol) of 8-[(2,6-difluorobenzyl)oxy]-2,6-dimethylimidazo[1,2-a]pyrazine-3-carboxylic acid from Example 3A together with 75 mg (0.20 mmol) of HATU and 0.094 ml (0.54 mmol) of N,N-diisopropylethylamine in 0.53 ml of DMF was first stirred for 10 min, then 55 mg (0.19 mmol, 85%) of ent-benzyl (1-amino-3-fluoro-2-methylpropan-2-yl)carbamate (enantiomer B) from Example 32A were added and the mixture was stirred at RT for 2.5 h. The reaction solution was admixed with acetonitrile, water and TFA and purified by means of preparative HPLC (RP18 column, eluent: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The aqueous phase was extracted twice with dichloromethane. The combined organic phases were dried over sodium sulphate, filtered, concentrated and lyophilized. 62 mg of the target compound (62% of theory) were obtained.

LC-MS (Method 13): Rₜ=1.55 min

MS (ESpos): m/z=556 (M+H)⁺

### Example 67A

### ent-Benzyl {1-[({8-[(2,6-difluorobenzyl)oxy]-2,6-dimethylimidazo[1,2-a]pyrazin-3-yl}carbonyl)amino]-5,5,5-trifluoro-2-methylpentan-2-yl}carbamate (enantiomer B)

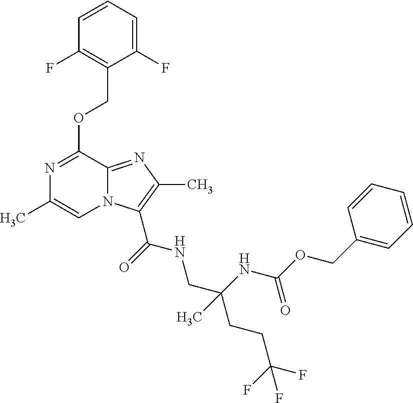 [SMILES: Cc1cn2c(C(=O)NCC(C)(CCC(F)(F)F)NC(=O)OCc3ccccc3)c(C)nc2c(OCc2c(F)cccc2F)n1]

An initial charge of 60 mg (0.18 mmol) of 8-[(2,6-difluorobenzyl)oxy]-2,6-dimethylimidazo[1,2-a]pyrazine-3-carboxylic acid from Example 3A together with 75 mg (0.20 mmol) of HATU and 0.094 ml (0.54 mmol) of N,N-diisopropylethylamine in 0.53 ml of DMF was stirred for 15 min. Subsequently, 69 mg (0.22 mmol; about 95% purity) of ent-benzyl (1-amino-5,5,5-trifluoro-2-methylpentan-2-yl)carbamate (enantiomer B) from Example 38A were added and the mixture was stirred at RT for 2.5 h. The reaction solution was admixed with acetonitrile, water and TFA and purified by means of preparative HPLC (RP18 column, eluent: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The aqueous phase was extracted twice with dichloromethane. The combined organic phases were dried over sodium sulphate, filtered, concentrated and lyophilized. 86 mg of the target compound (77% of theory) were obtained.

LC-MS (Method 2): Rₜ=1.34 min

MS (ESpos): m/z=620 (M+H)⁺

### Example 68A

### ent-Benzyl {1-[({8-[(2,6-difluorobenzyl)oxy]-2,6-dimethylimidazo[1,2-a]pyrazine-3-yl}carbonyl)amino]-5-fluoro-2-methylpentan-2-yl}carbamate trifluoroacetate (enantiomer B)

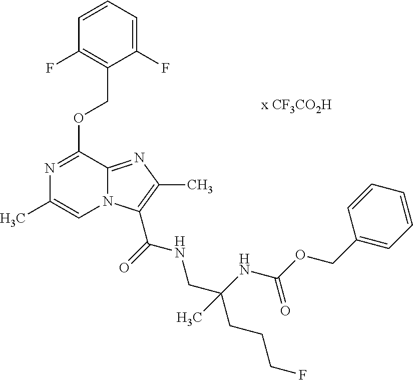 [SMILES: Cc1cn2c(C(=O)NCC(C)(CCCF)NC(=O)OCc3ccccc3)c(C)nc2c(OCc2c(F)cccc2F)n1]

An initial charge of 80 mg (0.24 mmol) of 8-[(2,6-difluorobenzyl)oxy]-2,6-dimethylimidazo[1,2-a]pyrazine-3-carboxylic acid from Example 3A together with 119 mg (0.31 mmol) of HATU and 0.21 ml (1.20 mmol) of N,N-diisopropylethylamine in 0.80 ml of DMF was stirred for 20 min. Subsequently, 91 mg (0.31 mmol; 93% purity) of ent-benzyl (1-amino-5-fluoro-2-methylpentan-2-yl)carbamate (enantiomer B) from Example 63A were added and the mixture was stirred at RT for 0.5 h. The reaction solution was admixed with acetonitrile, water and TFA and purified by means of preparative HPLC (RP18 column, eluent: acetonitrile/water gradient with addition of 0.1% TFA). 109 mg of the target compound were obtained (56% of theory, about 86% purity).

LC-MS (Method 2): Rₜ=1.28 min

MS (ESpos): m/z=584 (M-TFA+H)⁺

### Example 69A

### ent-Benzyl {1-[({8-[(2,6-difluorobenzyl)oxy]-2,6-dimethylimidazo[1,2-a]pyrazine-3-yl}carbonyl)amino]-4,4-difluoro-2-methylbutan-2-yl}carbamate (enantiomer A)

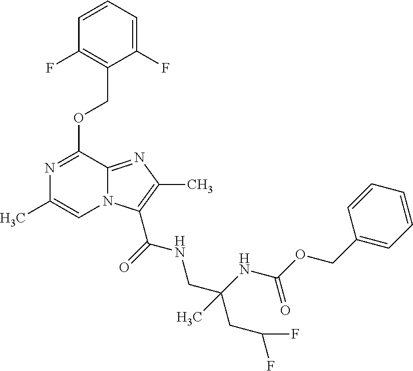 [SMILES: Cc1cn2c(C(=O)NCC(C)(CC(F)F)NC(=O)OCc3ccccc3)c(C)nc2c(OCc2c(F)cccc2F)n1]

An initial charge of 80 mg (0.24 mmol) of 8-[(2,6-difluorobenzyl)oxy]-2,6-dimethylimidazo[1,2-a]pyrazine-3-carboxylic acid from Example 3A together with 119 mg (0.31 mmol) of HATU and 0.21 ml (1.20 mmol) of N,N-diisopropylethylamine in 0.80 ml of DMF was stirred for 20 min. Subsequently, 85 mg (0.30 mmol; 98% purity) of ent-benzyl (1-amino-4,4-difluoro-2-methylbutan-2-yl)carbamate (enantiomer A) from Example 60A were added and the mixture was stirred at RT for 0.5 h. The reaction solution was admixed with water and stirred at RT for 30 min. The solid obtained was filtered off, washed well with water and dried. 127 mg of the target compound (90% of theory) were obtained.

LC-MS (Method 14): Rₜ=4.07 min

MS (ESpos): m/z=588 (M+H)⁺

### Example 70A

### ent-Benzyl {1-[({8-[(2,6-difluorobenzyl)oxy]-2,6-dimethylimidazo[1,2-a]pyrazine-3-yl}carbonyl)amino]-4,4-difluoro-2-methylbutan-2-yl}carbamate (enantiomer B)

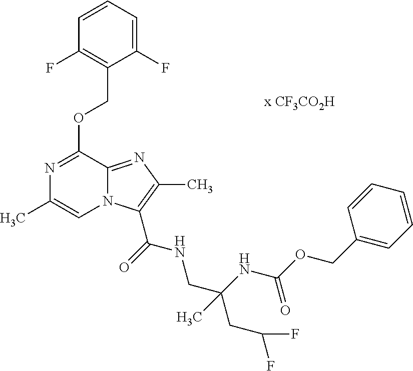 [SMILES: Cc1cn2c(C(=O)NCC(C)(CC(F)F)NC(=O)OCc3ccccc3)c(C)nc2c(OCc2c(F)cccc2F)n1]

An initial charge of 80 mg (0.24 mmol) of 8-[(2,6-difluorobenzyl)oxy]-2,6-dimethylimidazo[1,2-a]pyrazine-3-carboxylic acid from Example 3A together with 119 mg (0.31 mmol) of HATU and 0.21 ml (1.20 mmol) of N,N-diisopropylethylamine in 0.80 ml of DMF was stirred for 20 min. Subsequently, 85 mg (0.29 mmol; 93% purity) of ent-benzyl (1-amino-4,4-difluoro-2-methylbutan-2-yl)carbamate (enantiomer B) from Example 61A were added and the mixture was stirred at RT for 0.5 h. The reaction solution was admixed with water and stirred at RT for 30 min. The solid obtained was filtered off, washed well with water and purified by means of preparative HPLC (RP18 column, eluent: acetonitrile/water gradient with addition of 0.1% TFA). 93 mg of the target compound (55% of theory) were obtained.

LC-MS (Method 2): Rₜ=1.22 min

MS (ESpos): m/z=588 (M-TFA+H)⁺

### Example 71A

### Ethyl 8-chloro-2-methylimidazo[1,2-a]pyrazine-3-carboxylate

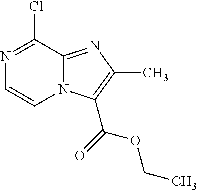 [SMILES: CCOC(=O)c1c(C)nc2c(Cl)nccn12]

Under argon, 1 g (7.72 mmol) of 3-chloropyrazin-2-amine was dissolved in 35 ml of ethanol, and 6.35 g (38.6 mmol) of ethyl 2-chloro-3-oxobutanoate were added. The reaction mixture was stirred under reflux for about 40 h. The mixture was cooled and concentrated by evaporation (dry ice cooling; oil pump at about 0.4 mbar; water bath temperature 60° C.). The residue was admixed with acetonitrile and extracted by stirring. The filtrate was concentrated by evaporation and purified by means of silica gel chromatography (eluent: cyclohexane/ethyl acetate gradient). 140 mg of the target compound (6% of theory) were obtained.

LC-MS (Method 16): Rₜ=1.70 min

MS (ESpos): m/z=240 (M+H)⁺

### Example 72A

### 8-Chloro-2-methylimidazo[1,2-a]pyrazine-3-carboxylic acid

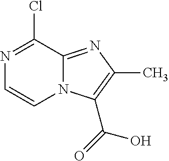

140 mg (0.47 mmol) of ethyl 8-chloro-2-methylimidazo[1,2-a]pyrazine-3-carboxylate from Example 71A were dissolved in 1.9 ml of 1,4-dioxane, 1.9 ml of 2 N sodium hydroxide solution were added and the mixture was stirred at RT overnight. The reaction mixture was acidified with 6 N hydrochloric acid and the mixture was concentrated. A little water was added to the residue, which was stirred briefly, and then the precipitated solid was filtered off. 68 mg of the target compound (68% of theory) were obtained.

LC-MS (Method 2): Rₜ=0.50 min

MS (ESpos): m/z=212 (M+H)⁺

### Example 73A

### 8-Chloro-N-(3,4-difluorobenzyl)-2-methylimidazo[1,2-a]pyrazine-3-carboxamide

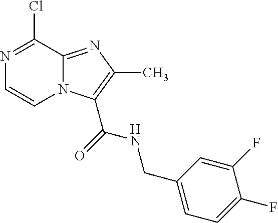 [SMILES: Cc1nc2c(Cl)nccn2c1C(=O)NCc1ccc(F)c(F)c1]

To a solution of 65 mg (0.31 mmol) of 8-chloro-2-methylimidazo[1,2-a]pyrazine-3-carboxylic acid from Example 72A in 3.5 ml of dichloromethane and 0.1 ml of DMF were added 99 mg (0.31 mmol) of (benzotriazol-1-yloxy)bisdimethylaminomethylium fluoroborate (TBTU), 44 mg (0.31 mmol) of 1-(3,4-difluorophenyl)methanamine and 0.17 ml (1.54 mmol) of 4-methylmorpholine, and the mixture was stirred at RT overnight. 2 ml of water were added to the mixture, which was stirred briefly and then filtered through an Extrelut cartridge. The cartridge was washed through well with dichloromethane/ethyl acetate and the filtrate was concentrated by evaporation and the residue was purified by means of preparative HPLC (Macherey-Nagel, VP50/21 Nucleodur C18 Gravity, 5 μm, 21×50 mm, eluent: acetonitrile/water gradient with addition of 0.1% conc. aqueous ammonia solution). 50 mg of the target compound (49% of theory) were obtained.

LC-MS (Method 2): Rₜ=0.91 min

MS (ESpos): m/z=337 (M+H)⁺

¹H-NMR (400 MHz, DMSO-d₆): δ [ppm]=2.67 (s, 3H), 4.54 (d, 2H), 7.21-7.28 (m, 1H), 7.36-7.49 (m, 2H), 7.84 (d, 1H), 8.74 (t, 1H), 8.92 (d, 1H).

### Example 74A

### 3-(Cyclobutylmethoxy)pyrazin-2-amine

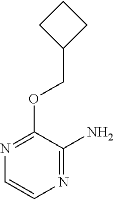 [SMILES: Nc1nccnc1OCC1CCC1]

To a mixture of 1.05 g (26.25 mmol; 60% purity) of sodium hydride in 15 ml of DMF were added 1.33 g (15.44 mmol) of cyclobutylmethanol and the mixture was stirred at RT for 1 h. Subsequently, a mixture of 1.0 g (7.72 mmol) of 3-chloropyrazin-2-amine in 10 ml DMF was added thereto and the reaction mixture was heated to 100° C. After 20 h, water was added to the mixture, which was extracted repeatedly with ethyl acetate. The combined organic phases were washed with saturated sodium chloride solution, dried over sodium sulphate, filtered and concentrated by evaporation. The residue obtained was purified by means of silica gel chromatography (eluent: cyclohexane/ethyl acetate gradient). 1.25 g of the title compound were obtained (89% of theory; 98% purity).

LC-MS (Method 2): Rₜ=0.80 min

MS (ESpos): m/z=180 (M+H)⁺

### Example 75A

### Ethyl 8-(cyclobutylmethoxy)-2-methylimidazo[1,2-a]pyrazine-3-carboxylate

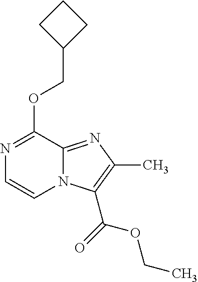 [SMILES: CCOC(=O)c1c(C)nc2c(OCC3CCC3)nccn12]

Under argon, 150 mg (0.84 mmol) of 3-(cyclobutylmethoxy)pyrazin-2-amine from Example 74A were dissolved in 6 ml of ethanol, and 689 mg (38.6 mmol) of ethyl 2-chloro-3-oxobutanoate were added. The reaction mixture was stirred under reflux for about 48 h. The mixture was cooled and concentrated by evaporation (dry ice cooling; oil pump at about 0.4 mbar; water bath temperature 60° C.). The residue was taken up in a little ethyl acetate and purified by means of silica gel chromatography (eluent: cyclohexane/ethyl acetate gradient). 75 mg of the target compound (30% of theory) were obtained.

LC-MS (Method 2): Rₜ=1.19 min

MS (ESpos): m/z=290 (M+H)⁺

¹H-NMR (400 MHz, DMSO-d₆): δ [ppm]=1.37 (t, 3H), 1.80-1.98 (m, 4H), 2.05-2.16 (m, 2H), 2.62 (s, 3H), 2.75-2.88 (m, 1H), 4.39 (q, 2H), 4.45 (d, 2H), 7.63 (d, 1H), 8.68 (d, 1H).

### Example 76A

### 8-(Cyclobutylmethoxy)-2-methylimidazo[1,2-a]pyrazine-3-carboxylic acid

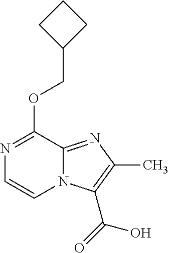 [SMILES: Cc1nc2c(OCC3CCC3)nccn2c1C(=O)O]

72 mg (0.25 mmol) of ethyl 8-(cyclobutylmethoxy)-2-methylimidazo[1,2-a]pyrazine-3-carboxylate from Example 75A were dissolved in 1.5 ml of 1,4-dioxane, 1 ml of 2 N sodium hydroxide solution were added and the mixture was stirred at RT for 3 h. The reaction mixture was acidified with 6 N hydrochloric acid, dichloromethane was added and the mixture was filtered through an Extrelut cartridge. The cartridge was washed through well with dichloromethane/ethyl acetate. The filtrate was concentrated by evaporation and the product was dried under high vacuum. 55 mg of the target compound (85% of theory) were obtained.

LC-MS (Method 1): Rₜ=0.96 min

MS (ESpos): m/z=262 (M+H)⁺

### WORKING EXAMPLES

### Example 1

### ent-N-[(2S)-2-Amino-2-methylbutyl]-8-[(2,6-difluorobenzyl)oxy]-2,6-dimethylimidazo[1,2-a]pyrazine-3-carboxamide

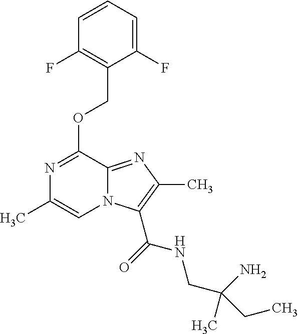 [SMILES: CCC(C)(N)CNC(=O)c1c(C)nc2c(OCc3c(F)cccc3F)nc(C)cn12]

A mixture of 74.0 mg of ent-benzyl {1-[({8-[(2,6-difluorobenzyl)oxy]-2,6-dimethylimidazo[1,2-a]pyrazin-3-yl}carbonyl)amino]-2-methylbutan-2-yl}carbamate (0.134 mmol, 1.0 eq.) from Example 9A and 9.4 mg of 20% palladium hydroxide on activated carbon (0.01 mmol, 0.1 eq) in ethanol was hydrogenated at standard pressure for 2 h. Subsequently, the mixture was filtered through kieselguhr and washed through, and the filtrate was concentrated. The crude product was purified by preparative HPLC (Method 6), giving 39 mg of the title compound (66% of theory).

LC-MS (Method 2): Rₜ=0.81 min

MS (ESpos): m/z=418 (M+H)⁺

¹H-NMR (400 MHz, DMSO-d₆): δ [ppm]=0.86 (t, 3H), 0.97 (s, 3H), 1.24-1.53 (m, 4H), 2.37 (s, 3H), 2.54 (s, 3H hidden beneath solvent signal), 3.14-3.27 (m, 2H), 5.54 (s, 2H), 7.16-7.28 (m, 2H), 7.50-7.63 (m, 1H), 7.69-8.01 (br s, 1H), 8.38 (d, 1H).

### Example 2

### 8-[(2,6-Difluorobenzyl)oxy]-N-[(2S)-hexan-2-yl]-2,6-dimethylimidazo[1,2-a]pyrazine-3-carboxamide

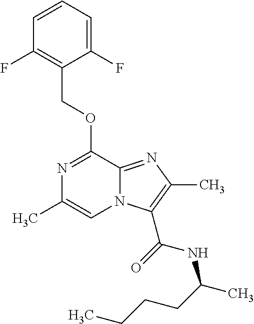 [SMILES: CCCC[C@H](C)NC(=O)c1c(C)nc2c(OCc3c(F)cccc3F)nc(C)cn12]

To a mixture of 60.0 mg of 8-[(2,6-difluorobenzyl)oxy]-2,6-dimethylimidazo[1,2-a]pyrazine-3-carboxylic acid (0.180 mmol, 1.0 eq.) from Example 3A, 27.0 mg of (2S)-hexan-2-amine [CAS No.: 70492-67-0] (0.270 mmol, 1.5 eq.) and 0.16 ml of N,N-diisopropylethylamine (0.90 mmol, 5 eq.) in 0.60 ml of DMF were added 89.0 mg of HATU (0.234 mmol, 1.3 eq) and the mixture was stirred at RT overnight. This was followed by extraction by stirring with water, removal of the solids by filtration, washing with water and drying under high vacuum. 46 mg of the title compound were obtained (58% of theory).

LC-MS (Method 2): Rₜ=1.31 min

MS (ESpos): m/z=417 (M+H)⁺

¹H-NMR (400 MHz, DMSO-d₆): δ [ppm]=0.83-0.92 (m., 3H), 1.18 (d, 3H), 1.22-1.63 (m, 6H), 2.37 (s, 3H), 2.49 (s, 3H masked by solvent signal), 3.76-4.25 (m, 1H), 5.54 (s, 2H), 7.17-7.26 (m, 2H), 7.48-7.67 (m, 1H), 7.90-7.96 (m, 1H), 8.26 (s, 1H).

### Example 3

### 8-[(2,6-Difluorobenzyl)oxy]-N-[(2R)-1-hydroxyhexan-2-yl]-2,6-dimethylimidazo[1,2-a]pyrazine-3-carboxamide

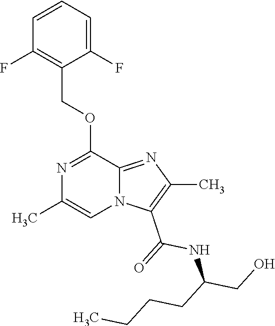 [SMILES: CCCC[C@H](CO)NC(=O)c1c(C)nc2c(OCc3c(F)cccc3F)nc(C)cn12]

To a mixture of 60.0 mg of 8-[(2,6-difluorobenzyl)oxy]-2,6-dimethylimidazo[1,2-a]pyrazine-3-carboxylic acid (0.180 mmol, 1.0 eq.) from Example 3A, 31.6 mg of (2R)-2-aminohexan-1-ol [CAS No.: 80696-28-2] (0.27 mmol, 1.5 eq.) and 0.16 ml of N,N-diisopropylethylamine (0.90 mmol, 5 eq.) in 0.60 ml of DMF were added 89.0 mg of HATU (0.234 mmol, 1.3 eq) and the mixture was stirred at RT overnight. This was followed by extraction by stirring with water, removal of the solids by filtration, washing with water and drying under high vacuum. 59.0 mg of the title compound were obtained (72% of theory).

LC-MS (Method 2): Rₜ=1.12 min

MS (ESpos): m/z=433 (M+H)⁺

¹H-NMR (400 MHz, DMSO-d₆): δ [ppm]=0.80-0.93 (m, 3H), 1.20-1.70 (m, 6H), 2.37 (s, 3H), 2.54 (s, 3H hidden beneath solvent signal), 3.39-3.55 (m, 2H), 3.88-4.03 (m, 1H), 4.65-4.83 (m, 1H), 5.54 (s, 2H), 7.17-7.26 (m, 2H), 7.46-7.63 (m, 1H), 7.71-7.83 (m, 1H), 8.27 (s, 1H).

### Example 4

### 8-[(2,6-Difluorobenzyl)oxy]-N-[1-(3,4-difluorophenyl)cyclopropyl]-2,6-dimethylimidazo[1,2-a]pyrazine-3-carboxamide

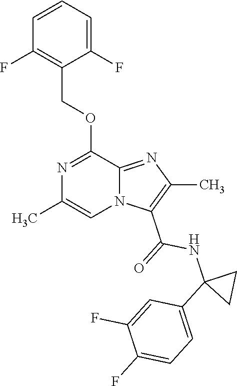 [SMILES: Cc1cn2c(C(=O)NC3(c4ccc(F)c(F)c4)CC3)c(C)nc2c(OCc2c(F)cccc2F)n1]

To a mixture of 60.0 mg of 8-[(2,6-difluorobenzyl)oxy]-2,6-dimethylimidazo[1,2-a]pyrazine-3-carboxylic acid (0.180 mmol, 1.0 eq.) from Example 3A, 45.7 mg of 1-(3,4-difluorophenyl)cyclopropanamine [CAS No.: 474709-85-8] (0.27 mmol, 1.5 eq.) and 0.16 ml of N,N-diisopropylethylamine (0.90 mmol, 5 eq.) in 0.60 ml of DMF were added 89.0 mg of HATU (0.234 mmol, 1.3 eq) and the mixture was stirred at RT overnight. This was followed by extraction by stirring with water, removal of the solids by filtration, washing with water and drying under high vacuum. 61.0 mg of the title compound were obtained (69% of theory).

LC-MS (Method 2): Rₜ=1.26 min

MS (ESpos): m/z=485 (M+H)⁺

¹H-NMR (400 MHz, DMSO-d₆): δ [ppm]=1.33 (s, 4H), 2.36 (s, 3H), 2.54 (s, 3H hidden beneath solvent signal), 5.54 (s, 2H), 7.07-7.42 (m, 5H), 7.51-7.66 (m, 1H), 8.30 (s, 1H), 8.81 (s, 1H).

### Example 5

### rac-N-(2-Amino-2,4-dimethylpentyl)-8-[(2,6-difluorobenzyl)oxy]-2,6-dimethylimidazo[1,2-a]pyrazine-3-carboxamide

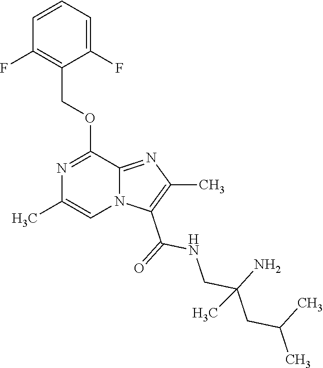 [SMILES: Cc1cn2c(C(=O)NCC(C)(N)CC(C)C)c(C)nc2c(OCc2c(F)cccc2F)n1]

An initial charge of 70 mg (0.21 mmol) of 8-[(2,6-difluorobenzyl)oxy]-2,6-dimethylimidazo[1,2-a]pyrazine-3-carboxylic acid from Example 3A together with 96 mg (0.25 mmol) of HATU and 0.18 ml (1.05 mmol) of N,N-diisopropylethylamine in 2.1 ml of DMF was stirred for 10 min, then 33 mg (0.25 mmol) of rac-2,4-dimethylpentane-1,2-diamine were added at RT and the mixture was stirred at RT overnight. The reaction mixture was extracted with dichloromethane, and the organic phase was dried over sodium sulphate, filtered and concentrated. The residue was purified by means of preparative HPLC (RP18 column, eluent: acetonitrile/water gradient with addition of 0.1% TFA). The product fraction was washed with saturated aqueous sodium hydrogencarbonate solution and dichloromethane, dried over sodium sulphate, filtered, concentrated and lyophilized. 69 mg of the target compound (72% of theory) were obtained.

LC-MS (Method 2): Rₜ=0.87 min

MS (ESpos): m/z=446 (M+H)⁺

¹H-NMR (400 MHz, DMSO-d₆): δ=0.91 (d, 3H), 0.94 (d, 3H), 1.02 (s, 3H), 1.20-1.33 (m, 2H), 1.51 (br. s, 2H), 1.75-1.88 (m, 1H), 2.36 (s, 3H), 2.55 (s, 3H masked by solvent peak), 3.21 (q, 2H), 5.54 (s, 2H), 7.17-7.27 (m, 2H), 7.51-7.62 (m, 1H), 7.76-7.96 (m, 1H), 8.38 (s, 1H).

### Example 6

### ent-8[(2,6-Difluorobenzyl)oxy]-2,6-dimethyl-N-(7,7,7-trifluoro-2-hydroxy-2-methylheptan-3-yl)imidazo[1,2-a]pyrazine-3-carboxamide

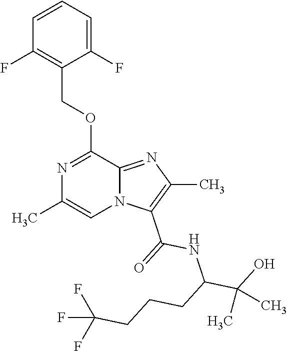 [SMILES: Cc1cn2c(C(=O)NC(CCCC(F)(F)F)C(C)(C)O)c(C)nc2c(OCc2c(F)cccc2F)n1]

An initial charge of 70 mg (0.21 mmol) of 8-[(2,6-difluorobenzyl)oxy]-2,6-dimethylimidazo[1,2-a]pyrazine-3-carboxylic acid from Example 3A together with 96 mg (0.25 mmol) of HATU and 0.18 ml (1.05 mmol) of N,N-diisopropylethylamine in 2.1 ml of DMF was stirred for 10 min, then 50 mg (0.25 mmol) of ent-3-amino-7,7,7-trifluoro-2-methylheptan-2-ol from Example 19A were added at RT and the mixture was stirred at RT overnight. The reaction mixture was extracted with dichloromethane, and the organic phase was dried over sodium sulphate, filtered and concentrated. The residue was purified by means of preparative HPLC (RP18 column, eluent: acetonitrile/water gradient with addition of 0.1% TFA). The product fraction was washed with saturated aqueous sodium hydrogencarbonate solution and dichloromethane, dried over sodium sulphate, filtered, concentrated and lyophilized. 98 mg of the target compound (86% of theory) were obtained.

LC-MS (Method 2): Rₜ=1.18 min

MS (ESpos): m/z=515 (M+H)⁺

¹H-NMR (400 MHz, DMSO-d₆): δ=1.12 (s, 3H), 1.17 (s, 3H), 1.42-1.63 (m, 3H), 1.77-1.87 (m, 1H), 2.17-2.30 (m, 1H), 2.30-2.45 (m, 4H), 2.52 (s, 3H, hidden by solvent peak), 3.91-4.01 (m, 1H), 4.58 (s, 1H), 5.55 (s, 2H), 7.18-7.26 (m, 2H), 7.52-7.61 (m, 1H), 7.68 (d, 1H), 8.23 (s, 1H).

### Example 7

### ent-8[(2,6-Difluorobenzyl)oxy]-N-(2-hydroxy-2-methylheptan-3-yl)-2,6-dimethylimidazo[1,2-a]pyrazine-3-carboxamide

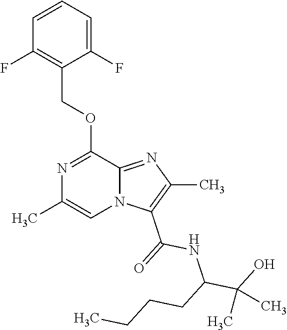 [SMILES: CCCCC(NC(=O)c1c(C)nc2c(OCc3c(F)cccc3F)nc(C)cn12)C(C)(C)O]

An initial charge of 70 mg (0.21 mmol) of 8-[(2,6-difluorobenzyl)oxy]-2,6-dimethylimidazo[1,2-a]pyrazine-3-carboxylic acid from Example 3A together with 96 mg (0.25 mmol) of HATU and 0.18 ml (1.05 mmol) of N,N-diisopropylethylamine in 2.1 ml of DMF was stirred for 10 min, then 36.6 mg (0.25 mmol) of ent-3-amino-2-methylheptan-2-ol from Example 13A were added at RT and the mixture was stirred at RT overnight. Another 24 mg (0.06 mmol) of HATU were added and the mixture was stirred at RT for 1.5 hours. Water and dichloromethane were added to the reaction solution. The phases were separated and the organic phase was dried over sodium sulphate, filtered and concentrated. The residue was purified by means of preparative HPLC (RP18 column, eluent: acetonitrile/water gradient with addition of 0.1% TFA). The product fraction was washed with saturated aqueous sodium hydrogencarbonate solution and dichloromethane, and the organic phase was dried over sodium sulphate, filtered, concentrated and lyophilized. 59 mg of the target compound (59% of theory) were obtained.

LC-MS (Method 2): Rₜ=1.20 min

MS (ESpos): m/z=461 (M+H)⁺

¹H-NMR (400 MHz, DMSO-d₆): δ=0.86 (t, 3H), 1.10 (s, 3H), 1.16 (s, 3H), 1.21-1.47 (m, 5H), 1.68-1.81 (m, 1H), 2.37 (s, 3H), 2.52 (s, 3H, hidden by solvent peak), 3.87-3.96 (m, 1H), 4.49 (s, 1H), 5.55 (s, 2H), 7.17-7.26 (m, 2H), 7.52-7.63 (m, 2H), 8.23 (s, 1H).

### Example 8

### Methyl trans-4-{[({8-[(2,6-difluorobenzyl)oxy]-2,6-dimethylimidazo yl}carbonyl)amino]methyl}cyclohexanecarboxylate

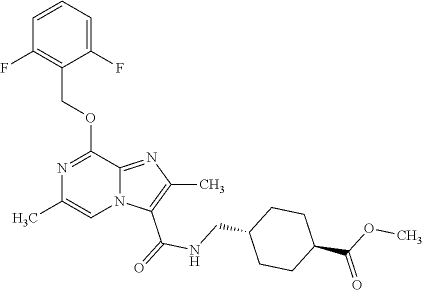 [SMILES: COC(=O)[C@H]1CC[C@H](CNC(=O)c2c(C)nc3c(OCc4c(F)cccc4F)nc(C)cn23)CC1]

An initial charge of 50 mg (0.15 mmol) of 8-[(2,6-difluorobenzyl)oxy]-2,6-dimethylimidazo[1,2-a]pyrazine-3-carboxylic acid from Example 3A together with 68.4 mg (0.18 mmol) of HATU and 0.13 ml (0.75 mmol) of N,N-diisopropylethylamine in 2 ml of DMF was stirred at RT for 10 min. Subsequently, 46.7 mg (0.23 mmol) of methyl trans-4-aminomethylcyclohexanecarboxylate hydrochloride were added to the reaction solution, which was stirred at RT overnight. The reaction mixture was purified by means of preparative HPLC (RP18 column; eluent: acetonitrile/water gradient with addition of 0.05% formic acid). 50 mg of the target compound (73% of theory) were obtained.

LC-MS (Method 2): Rₜ=1.18 min

MS (ESpos): m/z=487 (M+H)⁺

¹H-NMR (400 MHz, DMSO-d₆): δ=0.95-1.09 (m, 2H), 1.24-1.38 (m, 2H), 1.47-1.62 (m, 1H), 1.76-1.85 (m, 2H), 1.88-1.97 (m, 2H), 2.21-2.31 (m, 1H), 2.36 (s, 3H), 2.52 (s, 3H, masked by solvent peak), 3.17 (t, 2H), 3.58 (s, 3H), 5.54 (s, 2H), 7.17-7.26 (m, 2H), 7.52-7.61 (m, 1H), 8.08 (t, 1H), 8.32 (s, 1H).

### Example 9

### 8-[(2,6-Difluorobenzyl)oxy]-2,6-dimethyl-N-[(3S)-2-oxopyrrolidin-3-yl]imidazo[1,2-a]pyrazine-3-carboxamide

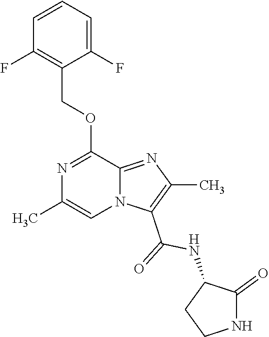 [SMILES: Cc1cn2c(C(=O)N[C@H]3CCNC3=O)c(C)nc2c(OCc2c(F)cccc2F)n1]

An initial charge of 50 mg (0.15 mmol) of 8-[(2,6-difluorobenzyl)oxy]-2,6-dimethylimidazo[1,2-a]pyrazine-3-carboxylic acid from Example 3A together with 68.4 mg (0.18 mmol) of HATU and 0.13 ml (0.75 mmol) of N,N-diisopropylethylamine in 2 ml of DMF was stirred at RT for 10 min. Subsequently, 22.5 mg (0.22 mmol) of (S)-3-aminopyrrolidin-2-one were added to the reaction solution, which was stirred at RT overnight. The reaction mixture was purified by means of preparative HPLC (RP18 column; eluent: acetonitrile/water gradient with addition of 0.05% formic acid). 50 mg of the target compound (80% of theory) were obtained.

LC-MS (Method 2): Rₜ=0.89 min

MS (ESpos): m/z=416 (M+H)⁺

¹H-NMR (400 MHz, DMSO-d₆): δ=1.98-2.11 (m, 1H), 2.37 (s, 3H), 2.39-2.44 (m, 1H), 2.52 (s, 3H), 3.25 (dd, 2H), 4.57 (q, 1H), 5.54 (s, 2H), 7.17-7.26 (m, 2H), 7.52-7.62 (m, 1H), 7.92 (s, 1H), 8.30 (d, 1H), 8.36 (s, 1H).

### Example 10

### 8-[(2,6-Difluorobenzyl)oxy]-N-(6-fluoroquinolin-4-yl)-2,6-dimethylimidazo[1,2-a]pyrazine-3-carboxamide

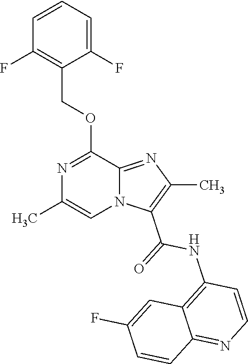 [SMILES: Cc1cn2c(C(=O)Nc3ccnc4ccc(F)cc34)c(C)nc2c(OCc2c(F)cccc2F)n1]

An initial charge of 50 mg (0.15 mmol) of 7-[(2,6-difluorobenzyl)oxy]-2,6-dimethylimidazo[1,2-a]pyrazine-3-carboxylic acid from Example 3A together with 74 mg (0.20 mmol) of HATU and 0.03 ml (0.30 mmol) of 4-methylmorpholine in 2 ml of DMF was stirred at RT for 30 min. Subsequently, 36.5 mg (0.23 mmol) of 6-fluoroquinolin-4-amine were added to the reaction solution, which was stirred at RT for 1 hour. The reaction mixture was purified by means of preparative HPLC (RP18 column; eluent: acetonitrile/water gradient with addition of 0.05% formic acid). 22 mg of the target compound (31% of theory) were obtained.

LC-MS (Method 2): Rₜ=1.09 min

MS (ESpos): m/z=478 (M+H)⁺

¹H-NMR (400 MHz, DMSO-d₆): δ=2.40 (s, 3H), 2.69 (s, 3H), 5.59 (s, 2H), 7.20-7.28 (m, 2H), 7.54-7.63 (m, 1H), 7.70-7.77 (m, 1H), 8.03-8.09 (m, 1H), 8.10-8.16 (m, 2H), 8.41 (s, 1H), 8.89 (d, 1H), 10.43 (s, 1H).

### Example 11

### trans-4-{[({8-[(2,6-Difluorobenzyl)oxy]-2,6-dimethylimidazo[1,2-a]pyrazin-3-yl}carbonyl)amino]methyl}cyclohexanecarboxylic acid hydrochloride

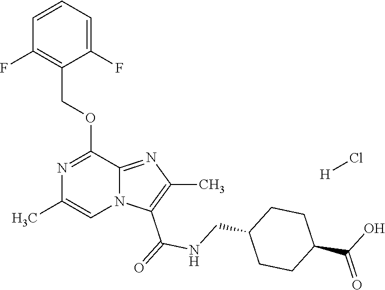

43 mg (0.088 mmol) of methyl trans-4-{[({8-[(2,6-difluorobenzyl)oxy]-2,6-dimethylimidazo[1,2-a]-pyrazin-3-yl}carbonyl)amino]methyl}cyclohexanecarboxylate from Example 8 were dissolved in 3 ml of THF/methanol (5/1), 0.44 ml (0.44 mmol) of a 1 N aqueous lithium hydroxide solution was added and the mixture was stirred at RT for 6 h. 0.44 ml (0.44 mmol) of a 1 N aqueous lithium hydroxide solution was added and the mixture was stirred at RT for 4 h. Then 0.44 ml (0.44 mmol) of a 1 N aqueous lithium hydroxide solution was added and the mixture was again stirred at RT for 4 h. Then 0.44 ml (0.44 mmol) of a 1 N aqueous lithium hydroxide solution was added once again and the mixture was stirred at RT for 4 h. The reaction solution was acidified with 1 N aqueous hydrochloric acid and the organic solvent was distilled off. The precipitate formed was filtered off, washed with water and dried under high vacuum. Subsequently, the residue was lyophilized. 31 mg of the target compound (65% of theory) were obtained.

LC-MS (Method 2): Rₜ=1.02 min

MS (ESpos): m/z=473 (M−HCl+H)⁺

¹H-NMR (500 MHz, DMSO-d₆): δ=0.95-1.06 (m, 2H), 1.24-1.36 (m, 2H), 1.48-1.61 (m, 1H), 1.77-1.84 (m, 2H), 1.88-1.96 (m, 2H), 2.10-2.19 (m, 1H), 2.37 (s, 3H), 2.52 (s, 3H, masked by solvent peak), 3.18 (t, 2H), 5.54 (s, 2H), 7.18-7.25 (m, 2H), 7.53-7.60 (m, 1H), 8.08 (t, 1H), 8.33 (s, 1H).

### Example 12

### 8-[(2,6-Difluorobenzyl)oxy]-N-[2-(1-hydroxycyclopentyl)ethyl]-2,6-dimethylimidazo[1,2-a]pyrazine-3-carboxamide

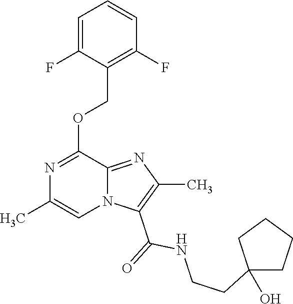 [SMILES: Cc1cn2c(C(=O)NCCC3(O)CCCC3)c(C)nc2c(OCc2c(F)cccc2F)n1]

12.9 mg (0.1 mmol) of 1-(2-aminoethyl)cyclopentanol were initially charged in a 96-well deep well multititre plate. A solution of 33.3 mg (0.1 mmol) of 8-[(2,6-difluorobenzyl)oxy]-2,6-dimethylimidazo[1,2-a]pyrazine-3-carboxylic acid from Example 3A in 0.3 ml of DMF and a solution of 45.6 mg (0.12 mol) of HATU in 0.3 ml of DMF were successively added thereto. After adding 20.2 mg (0.2 mmol) of 4-methylmorpholine, the mixture was shaken at RT overnight. Then the mixture was filtered and the target compound was isolated from the filtrate by preparative LC-MS (Method 9). The product-containing fractions were concentrated under reduced pressure using a centrifugal dryer. The residue of each product fraction was dissolved in 0.6 ml of DMSO. These were combined and finally freed of the solvent in a centrifugal dryer. 10.5 mg were obtained (21.5% of theory; 91% purity).

LC-MS (Method 10): Rₜ=1.10 min:

MS (Method 11): m/z=445 (M+H)⁺

In analogy to Example 12, the example compounds shown in Table 1 were prepared by reacting 8-[(2,6-difluorobenzyl)oxy]-2,6-dimethylimidazo[1,2-a]pyrazine-3-carboxylic acid from Example 3A with the appropriate amines, which are commercially available, known from the literature or described above, under the conditions described:

TABLE 1

| | IUPAC name/structure | |
|---|---|---|
| Example | (Yield) | Analytical data |
| 13 | 8-[(2,6-difluorobenzyl)oxy]-N-(2-hydroxy-2,3- | LC-MS (Method 10): Rₜ = |
| | dihydro-1H-inden-1-yl)-2,6-dimethylimidazo[1,2- | 1.12/1.13 min |
| | a]pyrazine-3-carboxamide | MS (ESneg): m/z = 463 (M − H)⁻ |
| | 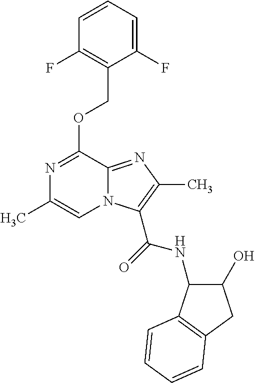 [SMILES: Cc1cn2c(C(=O)NC3c4ccccc4CC3O)c(C)nc2c(OCc2c(F)cccc2F)n1] | |
| | (32% of theory) | |
| 14 | rac-N-(1-cyanoethyl)-8-[(2,6-difluorobenzyl)oxy]- | LC-MS (Method 10): Rₜ = 1.08 min |
| | 2,6-dimethylimidazo[1,2-a]pyrazine-3- | MS (ESpos): m/z = 386 (M + H)⁺ |
| | carboxamide | |
| | 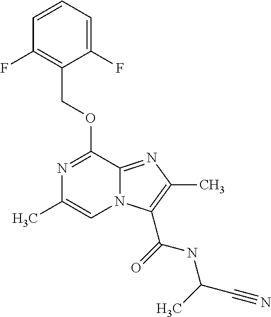 [SMILES: Cc1cn2c(C(=O)NC(C)C#N)c(C)nc2c(OCc2c(F)cccc2F)n1] | |
| | (35% of theory) | |
| 15 | N-(2-amino-2-ethylbutyl)-8-[(2,6- | LC-MS (Method 10): Rₜ = 0.78 min |
| | difluorobenzyl)oxy]-2,6-dimethylimidazo[1,2- | MS (ESpos): m/z = 432 (M + H)⁺ |
| | a]pyrazine-3-carboxamide | |
| | 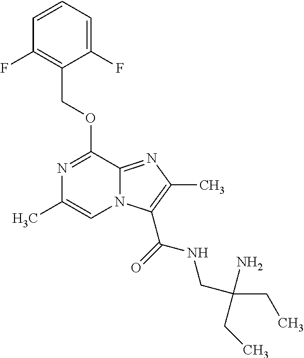 [SMILES: CCC(N)(CC)CNC(=O)c1c(C)nc2c(OCc3c(F)cccc3F)nc(C)cn12] | |
| | (8% of theory) | |
| 16 | 8-[(2,6-difluorobenzyl)oxy]-2,6-dimethyl-N- | LC-MS (Method 10): Rₜ = 0.78 min |
| | (2,2,6,6-tetramethylpiperidin-4-yl)imidazo[1,2- | MS (ESpos): m/z = 472 (M + H)⁺ |
| | a]pyrazine-3-carboxamide | |
| | 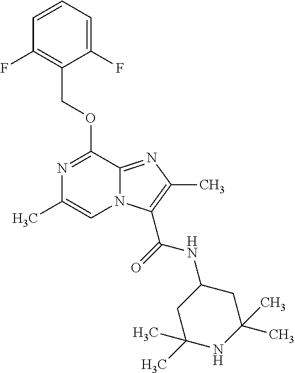 [SMILES: Cc1cn2c(C(=O)NC3CC(C)(C)NC(C)(C)C3)c(C)nc2c(OCc2c(F)cccc2F)n1] | |
| | (6% of theory) | |
| 17 | rac-8-[(2,6-difluorobenzyl)oxy]-2,6-dimethyl-N- | LC-MS (Method 10): Rₜ = 1.21 min |
| | (2-phenylcyclopropyl)imidazo[1,2-a]pyrazine-3- | MS (ESpos): m/z = 449 (M + H)⁺ |
| | carboxamide | |
| | 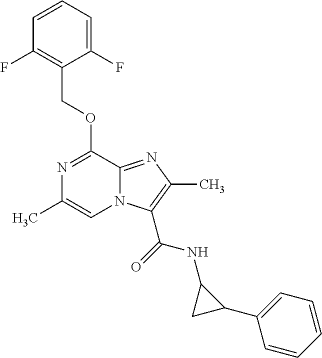 [SMILES: Cc1cn2c(C(=O)NC3CC3c3ccccc3)c(C)nc2c(OCc2c(F)cccc2F)n1] | |
| | (38% of theory) | |
| 18 | 8-[(2,6-difluorobenzyl)oxy]-N-[1-(2- | LC-MS (Method 10): Rₜ = 0.96 min |
| | hydroxyethyl)-3,5-dimethyl-1H-pyrazol-4-yl]-2,6- | MS (ESpos): m/z = 471 (M + H)⁺ |
| | dimethylimidazo[1,2-a]pyrazine-3-carboxamide | |
| | 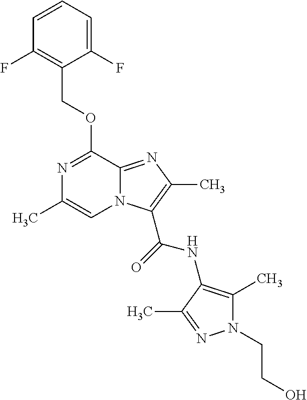 [SMILES: Cc1cn2c(C(=O)Nc3c(C)nn(CCO)c3C)c(C)nc2c(OCc2c(F)cccc2F)n1] | |
| | (22% of theory) | |
| 19 | 8-[(2,6-difluorobenzyl)oxy]-N-[2- | LC-MS (Method 10): Rₜ = 0.73 min |
| | (dimethylamino)ethyl]-2,6-dimethylimidazo[1,2- | MS (ESpos): m/z = 404 (M + H)⁺ |
| | a]pyrazine-3-carboxamide | |
| | 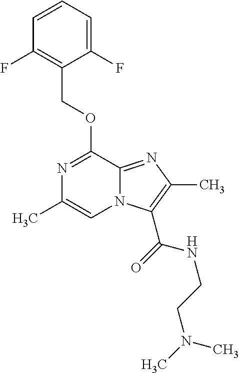 [SMILES: Cc1cn2c(C(=O)NCCN(C)C)c(C)nc2c(OCc2c(F)cccc2F)n1] | |
| | (6% of theory) | |
| 20 | rac-N-(2-amino-2-cyclopropylpropyl)-8-[(2,6- | LC-MS (Method 10): Rₜ = 0.76 min |
| | difluorobenzyl)oxy]-2,6-dimethylimidazo[1,2- | MS (ESpos): m/z = 430 (M + H)⁺ |
| | a]pyrazine-3-carboxamide | |
| | 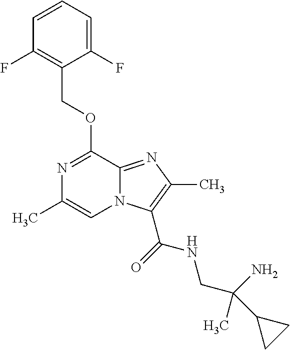 [SMILES: Cc1cn2c(C(=O)NCC(C)(N)C3CC3)c(C)nc2c(OCc2c(F)cccc2F)n1] | |
| | (8% of theory) | |
| 21 | 8-[(2,6-difluorobenzyl)oxy]-2,6-dimethyl-N-[2- | LC-MS (Method 10): Rₜ = 1.12 min |
| | (pyridin-3-yl)benzyl]imidazo[1,2-a]pyrazine-3- | MS (ESpos): m/z = 500 (M + H)⁺ |
| | carboxamide | |
| | 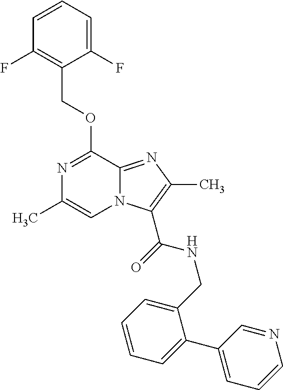 [SMILES: Cc1cn2c(C(=O)NCc3ccccc3-c3cccnc3)c(C)nc2c(OCc2c(F)cccc2F)n1] | |
| | (42% of theory) | |
| 22 | 8-[(2,6-difluorobenzyl)oxy]-2,6-dimethyl-N-[2- | LC-MS (Method 10): Rₜ = 0.74 min |
| | (morpholin-4-yl)ethyl]imidazo[1,2-a]pyrazine-3- | MS (ESpos): m/z = 446 (M + H)⁺ |
| | carboxamide | |
| | 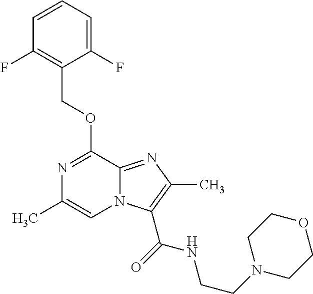 [SMILES: Cc1cn2c(C(=O)NCCN3CCOCC3)c(C)nc2c(OCc2c(F)cccc2F)n1] | |
| | (6% of theory) | |
| 23 | 8-[(2,6-difluorobenzyl)oxy]-N-[1-(4- | LC-MS (Method 10): Rₜ = 1.17 min |
| | fluorobenzyl)-3,5-dimethyl-1H-pyrazol-4-yl]-2,6- | MS (ESpos): m/z = 535 (M + H)⁺ |
| | dimethylimidazo[1,2-a]pyrazine-3-carboxamide | |
| | 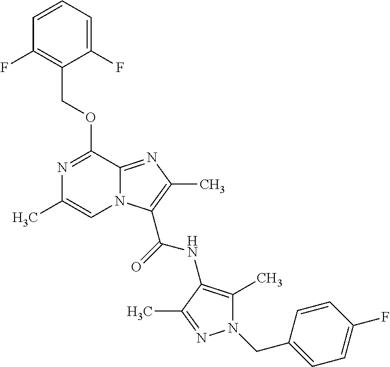 [SMILES: Cc1cn2c(C(=O)Nc3c(C)nn(Cc4ccc(F)cc4)c3C)c(C)nc2c(OCc2c(F)cccc2F)n1] | |
| | (27% of theory) | |
| 24 | 8-[(2,6-difluorobenzyl)oxy]-2,6-dimethyl-N-[1- | LC-MS (Method 10): Rₜ = 1.13 min |
| | (2,2,2-trifluoroethyl)-1H-pyrazol-4- | MS (ESpos): m/z = 481 (M + H)⁺ |
| | yl]imidazo[1,2-a]pyrazine-3-carboxamide | |
| | 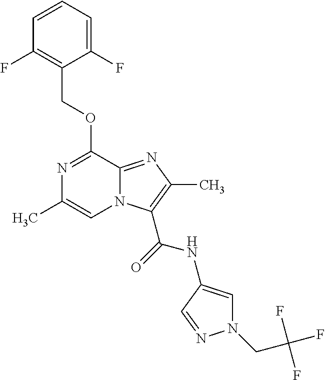 [SMILES: Cc1cn2c(C(=O)Nc3cnn(CC(F)(F)F)c3)c(C)nc2c(OCc2c(F)cccc2F)n1] | |
| | (32% of theory) | |
| 25 | 8-[(2,6-difluorobenzyl)oxy]-N-(1H-imidazol-2- | LC-MS (Method 10): Rₜ = 0.73 min |
| | ylmethyl)-2,6-dimethylimidazo[1,2-a]pyrazine-3- | MS (ESpos): m/z = 413 (M + H)⁺ |
| | carboxamide | |
| | 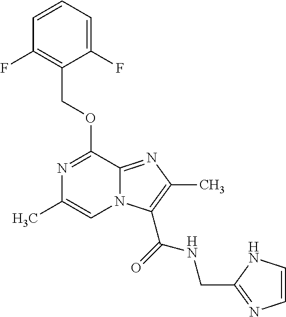 [SMILES: Cc1cn2c(C(=O)NCc3ncc[nH]3)c(C)nc2c(OCc2c(F)cccc2F)n1] | |
| | (11% of theory) | |
| 26 | rac-8-[(2,6-difluorobenzyl)oxy]-2,6-dimethyl-N- | LC-MS (Method 10): Rₜ = 1.21 min |
| | {1-[3-(trifluoromethyl)-1,2,4-oxadiazol-5- | MS (ESpos): m/z = 497 (M + H)⁺ |
| | yl]ethyl}imidazo[1,2-a]pyrazine-3-carboxamide | |
| | 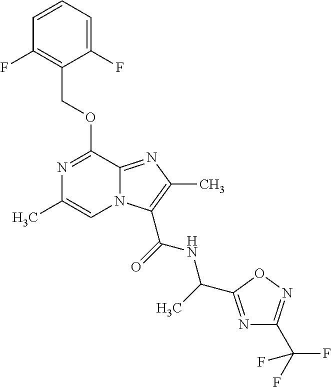 [SMILES: Cc1cn2c(C(=O)NC(C)c3nc(C(F)(F)F)no3)c(C)nc2c(OCc2c(F)cccc2F)n1] | |
| | (26% of theory) | |
| 27 | 8-[(2,6-difluorobenzyl)oxy]-2,6-dimethyl-N-[5- | LC-MS (Method 10): Rₜ = 1.07 min |
| | methyl-3-(trifluoromethyl)-1H-pyrazol-4- | MS (ESpos): m/z = 481 (M + H)⁺ |
| | yl]imidazo[1,2-a]pyrazine-3-carboxamide | |
| | 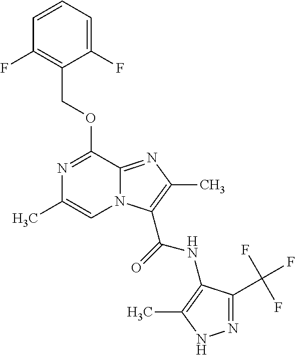 [SMILES: Cc1cn2c(C(=O)Nc3c(C(F)(F)F)n[nH]c3C)c(C)nc2c(OCc2c(F)cccc2F)n1] | |
| | (35% of theory) | |
| 28 | N-[(6-chloropyridin-3-yl)methyl]-8-[(2,6- | LC-MS (Method 10): Rₜ = 1.13 min |
| | difluorobenzyl)oxy]-2,6-dimethylimidazo[1,2- | MS (ESpos): m/z = 458 (M + H)⁺ |
| | a]pyrazine-3-carboxamide | |
| | 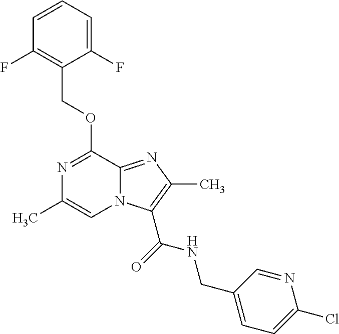 [SMILES: Cc1cn2c(C(=O)NCc3ccc(Cl)nc3)c(C)nc2c(OCc2c(F)cccc2F)n1] | |
| | (26% of theory) | |
| 29 | ent-8-[(2,6-difluorobenzyl)oxy]-2,6-dimethyl-N- | LC-MS (Method 10): Rₜ = 1.18 min |
| | (6,6,7,7,7-pentafluoro-2-hydroxy-2-methylheptan- | MS (ESpos): m/z = 551 (M + H)⁺ |
| | 3-yl)imidazo[1,2-a]pyrazine-3-carboxamide | |
| | 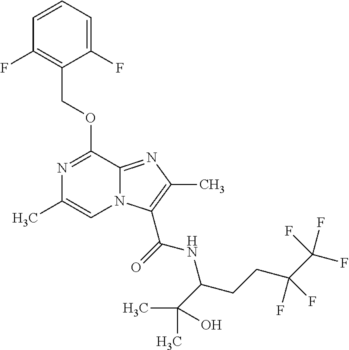 [SMILES: Cc1cn2c(C(=O)NC(CCC(F)(F)C(F)(F)F)C(C)(C)O)c(C)nc2c(OCc2c(F)cccc2F)n1] | |
| | (27% of theory) | |
| 30 | 8-[(2,6-difluorobenzyl)oxy]-2,6-dimethyl-N- | LC-MS (Method 10): Rₜ = 1.12 min |
| | (pyrazolo[1,5-a]pyridin-3-yl)imidazo[1,2- | MS (ESpos): m/z = 449 (M + H)⁺ |
| | a]pyrazine-3-carboxamide | |
| | 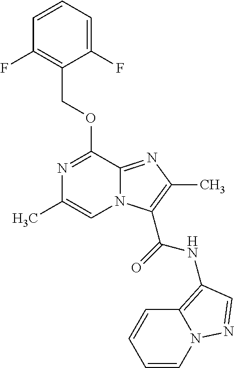 [SMILES: Cc1cn2c(C(=O)Nc3cnn4ccccc34)c(C)nc2c(OCc2c(F)cccc2F)n1] | |
| | (45% of theory) | |
| 31 | rac-8-[(2,6-difluorobenzyl)oxy]-2,6-dimethyl-N- | LC-MS (Method 10): Rₜ = 1.17 min |
| | [1-(2,2,2-trifluoroethoxy)propan-2-yl]imidazo[1,2- | MS (ESpos): m/z = 473 (M + H)⁺ |
| | a]pyrazine-3-carboxamide | |
| | 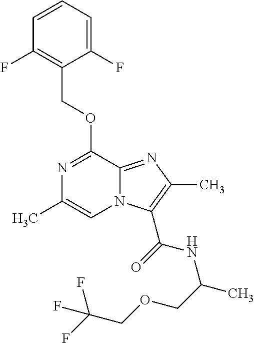 [SMILES: Cc1cn2c(C(=O)NC(C)COCC(F)(F)F)c(C)nc2c(OCc2c(F)cccc2F)n1] | |
| | (33% of theory) | |
| 32 | 8-[(2,6-difluorobenzyl)oxy]-N-[(1- | LC-MS (Method 10): Rₜ = 0.99 min |
| | hydroxycyclopropyl)methyl]-2,6- | MS (ESpos): m/z = 403 (M + H)⁺ |
| | dimethylimidazo[1,2-a]pyrazine-3-carboxamide | |
| | 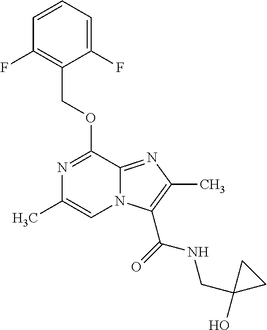 [SMILES: Cc1cn2c(C(=O)NCC3(O)CC3)c(C)nc2c(OCc2c(F)cccc2F)n1] | |
| | (31% of theory) | |
| 33 | N-{2-[cyclopropyl(2,2-difluoroethyl)amino]ethyl}- | LC-MS (Method 10): Rₜ = 1.19 min |
| | 8-[(2,6-difluorobenzyl)oxy]-2,6- | MS (ESpos): m/z = 480 (M + H)⁺ |
| | dimethylimidazo[1,2-a]pyrazine-3-carboxamide | |
| | 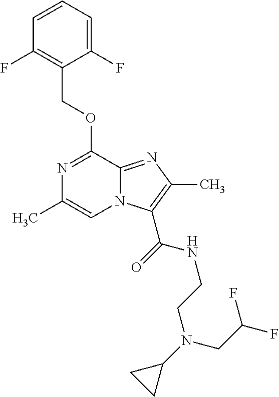 [SMILES: Cc1cn2c(C(=O)NCCN(CC(F)F)C3CC3)c(C)nc2c(OCc2c(F)cccc2F)n1] | |
| | (9% of theory) | |
| 34 | 8-[(2,6-difluorobenzyl)oxy]-2,6-dimethyl-N-[(3S)- | LC-MS (Method 10): Rₜ = 1.02 min |
| | 2-oxotetrahydrofuran-3-yl]imidazo[1,2-a]pyrazine- | MS (ESpos): m/z = 417 (M + H)⁺ |
| | 3-carboxamide | |
| | 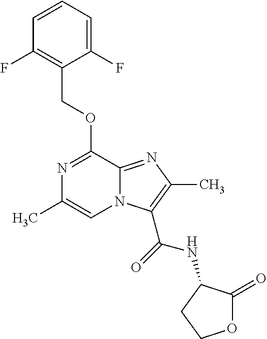 [SMILES: Cc1cn2c(C(=O)N[C@H]3CCOC3=O)c(C)nc2c(OCc2c(F)cccc2F)n1] | |
| | (34% of theory) | |
| 35 | 8-[(2,6-difluorobenzyl)oxy]-N-[1-hydroxy-2- | LC-MS (Method 10): Rₜ = 1.00 min |
| | (hydroxymethyl)butan-2-yl]-2,6- | MS (ESpos): m/z = 435 (M + H)⁺ |
| | dimethylimidazo[1,2-a]pyrazine-3-carboxamide | |
| | 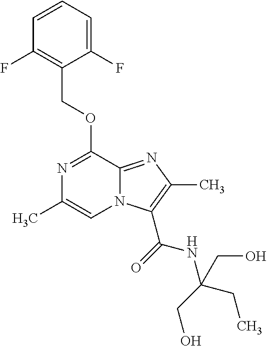 [SMILES: CCC(CO)(CO)NC(=O)c1c(C)nc2c(OCc3c(F)cccc3F)nc(C)cn12] | |
| | (32% of theory) | |
| 36 | 8-[(2,6-difluorobenzyl)oxy]-2,6-dimethyl-N-(9- | LC-MS (Method 10): Rₜ = 0.77 min |
| | methyl-9-azabicyclo[3.3.1]non-3-yl)imidazo[1,2- | MS (ESpos): m/z = 470 (M + H)⁺ |
| | a]pyrazine-3-carboxamide | |
| | 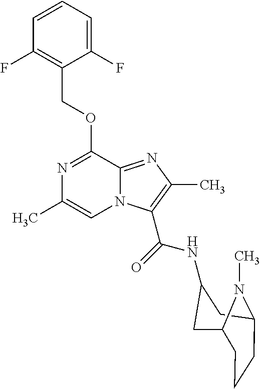 [SMILES: Cc1cn2c(C(=O)NC3CC4CCCC(C3)N4C)c(C)nc2c(OCc2c(F)cccc2F)n1] | |
| | (15% of theory) | |

### Example 37

### ent-N-(2-Amino-3-fluoro-2-methylpropyl)-8-[(2,6-difluorobenzyl)oxy]-2,6-dimethylimidazo[1,2-a]pyrazine-3-carboxamide (enantiomer B)

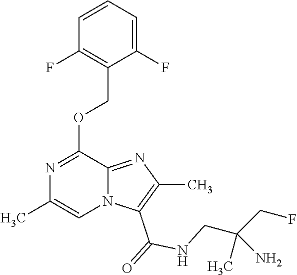 [SMILES: Cc1cn2c(C(=O)NCC(C)(N)CF)c(C)nc2c(OCc2c(F)cccc2F)n1]

Under argon, 62 mg (0.11 mmol) of ent-benzyl {-[({8-[(2,6-difluorobenzyl)oxy]-2,6-dimethylimidazo[1,2-a]pyrazin-3-yl}carbonyl)amino]-3-fluoro-2-methylpropan-2-yl}carbamate (enantiomer B) from Example 66A were dissolved in 2.9 ml of ethanol, 6 mg of palladium on activated carbon (10%) were added and the reaction mixture was hydrogenated at standard pressure at RT for 45 min. The reaction mixture was filtered through Celite and washed well with ethanol, and the filtrate was then concentrated. The residue was purified by thick-layer chromatography (eluent: dichloromethane/2 M ammonia solution in methanol=20/1). This gave 34 mg of the target compound (47% of theory, purity 98%).

LC-MS (Method 2): Rₜ=0.70 min

MS (ESpos): m/z=422 (M+H)⁺

¹H-NMR (500 MHz, DMSO-d₆): δ=1.03 (s, 3H), 1.68 (br. s, 2H), 2.37 (s, 3H), 2.51 (s, 3H; masked by solvent peak), 3.25-3.38 (m, 2H; masked by water peak), 4.08-4.15 (m, 1H), 4.18-4.25 (m, 1H), 5.53 (s, 2H), 7.18-7.25 (m, 2H), 7.54-7.62 (m, 1H), 7.87-7.94 (m, 1H), 8.38 (s, 1H).

### Example 38

### ent-N-(2-Amino-5,5,5-trifluoro-2-methylpentyl)-8-[(2,6-difluorobenzyl)oxy]-2,6-dimethylimidazo[1,2-a]pyrazine-3-carboxamide (enantiomer B)

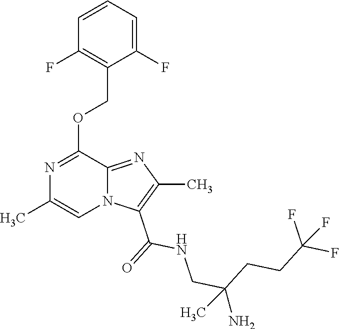 [SMILES: Cc1cn2c(C(=O)NCC(C)(N)CCC(F)(F)F)c(C)nc2c(OCc2c(F)cccc2F)n1]

A mixture of 86 mg (0.14 mmol) of ent-benzyl {1-[({8-[(2,6-difluorobenzyl)oxy]-2,6-dimethylimidazo[1,2-a]pyrazin-3-yl}carbonyl)amino]-5,5,5-trifluoro-2-methylpentan-2-yl}carbamate (enantiomer B) from Example 67A and 7 mg of palladium on activated carbon (10%) in 3.2 ml of ethanol was hydrogenated at room temperature and standard pressure for 1.5 h. Subsequently, the mixture was filtered through a Millipore filter and washed with ethanol, and the filtrate was concentrated. The crude product was admixed with acetonitrile, water and TFA and purified by means of preparative HPLC (RP-C18, eluent: acetonitrile/water gradient with addition of 0.1% TFA). The product fractions were taken up in dichloromethane and washed twice with saturated aqueous sodium hydrogencarbonate solution. The combined aqueous phases were extracted twice with dichloromethane. The combined organic phases were dried over sodium sulphate, filtered and concentrated by evaporation. 10 mg of the title compound were obtained (15% of theory; 98% purity).

LC-MS (Method 2): Rₜ=0.79 min

MS (ESpos): m/z=486 (M+H)⁺

¹H-NMR (500 MHz, DMSO-d₆): δ [ppm]=1.02 (s, 3H), 1.59-1.70 (m, 2H), 2.30-2.47 (m, 5H), 2.50 (s, 3H; masked by solvent peak), 3.20-3.40 (m, 2H; masked by water peak), 5.54 (s, 2H), 7.18-7.25 (m, 2H), 7.54-7.62 (m, 1H), 8.04-8.15 (m, 1H), 8.32 (s, 1H).

### Example 39

### ent-N-(2-Amino-2-methylpentyl)-8-[(2,6-difluorobenzyl)oxy]-2,6-dimethylimidazo[1,2-a]pyrazine-3-carboxamide (enantiomer B)

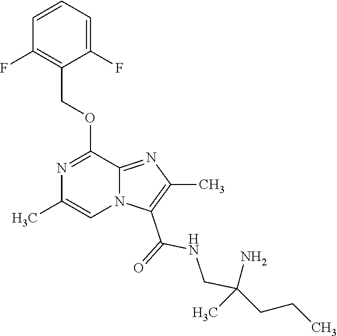 [SMILES: CCCC(C)(N)CNC(=O)c1c(C)nc2c(OCc3c(F)cccc3F)nc(C)cn12]

66 mg (0.10 mmol) of ent-benzyl {1-[({8-[(2,6-difluorobenzyl)oxy]-2,6-dimethylimidazo[1,2-a]-pyrazin-3-yl}carbonyl)amino]-2-methylpentan-2-yl}carbamate trifluoroacetate (enantiomer B) from Example 65A were dissolved in 2.5 ml of ethanol, 3.1 mg of 10% palladium on activated carbon were added and hydrogenation was effected at standard pressure for a total of 100 min. The reaction solution was filtered by means of a Millipore filter and the filtrate was concentrated by rotary evaporation. The residue was purified by means of preparative HPLC (RP18 column, eluent: acetonitrile/water gradient with addition of 0.1% TFA). The product fractions were combined and concentrated. All the product fractions were combined and concentrated. Subsequently, the residue was taken up in dichloromethane and a little methanol, and washed with a little saturated aqueous sodium hydrogencarbonate solution. The aqueous phase was re-extracted twice with dichloromethane. The combined organic phases were dried over sodium sulphate, filtered, concentrated and lyophilized. 31 mg of the target compound (73% of theory) were obtained.

LC-MS (Method 2): Rₜ=0.77 min

MS (ESpos): m/z=432 (M+H)⁺

¹H-NMR (400 MHz, DMSO-d₆): δ=0.86 (t, 3H), 0.99 (s, 3H), 1.20-1.42 (m, 4H), 1.70 (br. s, 2H), 2.35 (s, 3H), 2.55 (s, 3H; masked by solvent peak), 3.15-3.30 (m, 2H), 5.54 (s, 2H), 7.16-7.25 (m, 2H), 7.52-7.61 (m, 1H), 7.82 (br. s, 1H), 8.37 (s, 1H).

### Example 40

### ent-N-(2-Amino-5-fluoro-2-methylpentyl)-8-[(2,6-difluorobenzyl)oxy]-2,6-dimethylimidazo[1,2-a]pyrazine-3-carboxamide (enantiomer B)

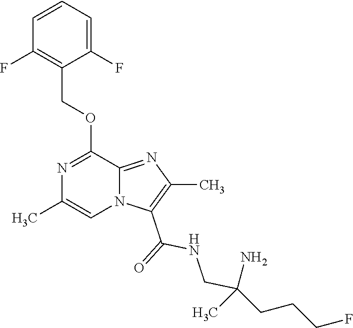 [SMILES: Cc1cn2c(C(=O)NCC(C)(N)CCCF)c(C)nc2c(OCc2c(F)cccc2F)n1]

109 mg (0.13 mmol, 86% purity) of ent-benzyl {1-[({(8-[(2,6-difluorobenzyl)oxy]-2,6-dimethylimidazo[1,2-a]pyrazin-3-yl}carbonyl)amino]-5-fluoro-2-methylpentan-2-yl}carbamate trifluoroacetate (enantiomer B) from Example 68A were dissolved in 3.4 ml of ethanol, 4.3 mg of 10% palladium on activated carbon were added and hydrogenation was effected at standard pressure for a total of 2.5 hours. The reaction solution was filtered by means of a Millipore filter and the filtrate was concentrated by rotary evaporation. The residue was purified by means of preparative HPLC (RP18 column, eluent: acetonitrile/water gradient with addition of 0.1% TFA). All the product fractions were combined and concentrated. Subsequently, the residue was taken up in dichloromethane and a little methanol, and washed twice with saturated aqueous sodium hydrogencarbonate solution. The aqueous phase was reextracted twice with dichloromethane. The combined organic phases were dried over sodium sulphate, filtered, concentrated and lyophilized. 42 mg of the target compound (68% of theory) were obtained.

LC-MS (Method 2): Rₜ=0.69 min

MS (ESpos): m/z=450 (M+H)⁺

¹H-NMR (400 MHz, DMSO-d₆): δ=1.00 (s, 3H), 1.32-1.88 (m, 8H), 2.35 (s, 3H), 2.54 (s, 3H; masked by solvent peak), 3.18-3.30 (m, 2H), 4.35 (t, 1H), 4.48 (t, 1H), 5.53 (s, 2H), 7.18-7.28 (m, 2H), 7.52-7.63 (m, 1H), 7.88 (br. s, 1H), 8.35 (s, 1H).

### Example 41

ent-N-(2-Amino-4,4-difluoro-2-methylbutyl)-8-[(2,6-difluorobenzyl)oxy]-2,6-dimethylimidazo[1,2-a]pyrazine-3-carboxamide (enantiomer A)

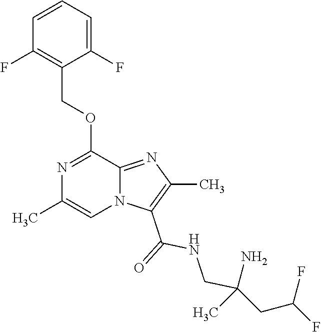 [SMILES: Cc1cn2c(C(=O)NCC(C)(N)CC(F)F)c(C)nc2c(OCc2c(F)cccc2F)n1]

127 mg (0.22 mmol) of ent-benzyl {1-[({8-[(2,6-difluorobenzyl)oxy]-2,6-dimethylimidazo[1,2-a]pyrazin-3-yl}carbonyl)amino]-4,4-difluoro-2-methylbutan-2-yl}carbamate (enantiomer A) from Example 69A were dissolved in 5.5 ml of ethanol, 33 μl of TFA and 7 mg of 10% palladium on activated carbon were added and hydrogenation was effected at standard pressure for 2.5 hours. The reaction solution was filtered by means of a Millipore filter and the filtrate was concentrated by rotary evaporation. The residue was purified by means of preparative HPLC (RP18 column, eluent: acetonitrile/water gradient with addition of 0.1% TFA). The product fractions were combined and concentrated. All the product fractions were combined and concentrated. Subsequently, the residue was taken up in dichloromethane and a little methanol, and washed with a little saturated aqueous sodium hydrogencarbonate solution. The aqueous phase was reextracted twice with dichloromethane. The combined organic phases were dried over sodium sulphate, filtered, concentrated and lyophilized. 75 mg of the target compound (75% of theory) were obtained.

LC-MS (Method 2): Rₜ=0.74 min

MS (ESpos): m/z=454 (M+H)⁺

¹H-NMR (500 MHz, DMSO-d₆): δ=1.08 (s, 3H), 1.72 (br. s, 2H), 1.87-1.99 (m, 2H), 2.36 (s, 3H), 2.56 (s, 3H), 3.22-3.32 (m, 2H; masked by solvent peak), 5.55 (s, 2H), 6.11-6.39 (m, 1H), 7.18-7.25 (m, 2H), 7.53-7.62 (m, 1H), 7.92-8.01 (m, 1H), 8.34 (s, 1H).

### Example 42

### ent-N-(2-Amino-4,4-difluoro-2-methylbutyl)-8-[(2,6-difluorobenzyl)oxy]-2,6-dimethylimidazo[1,2-a]pyrazine-3-carboxamide (enantiomer B)

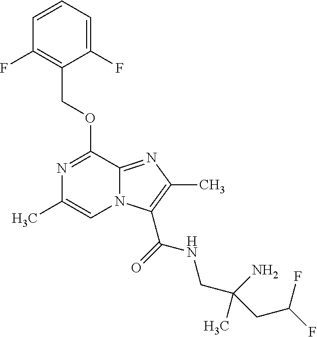 [SMILES: Cc1cn2c(C(=O)NCC(C)(N)CC(F)F)c(C)nc2c(OCc2c(F)cccc2F)n1]

93 mg (0.13 mmol) of ent-benzyl {1-[({8-[(2,6-difluorobenzyl)oxy]-2,6-dimethylimidazo[1,2-a]pyrazin-3-yl}carbonyl)amino]-4,4-difluoro-2-methylbutan-2-yl}carbamate trifluoroacetate (enantiomer B) from Example 70A were dissolved in 3.4 ml of ethanol, 4.2 mg of 10% palladium on activated carbon were added and hydrogenation was effected at standard pressure for 70 min. The reaction solution was filtered by means of a Millipore filter and the filtrate was concentrated by evaporation. The residue was purified by means of preparative HPLC (RP18 column, eluent: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was re-extracted twice with dichloromethane. The combined organic phases were dried over sodium sulphate, filtered, concentrated by evaporation and lyophilized. 47 mg of the target compound (78% of theory) were obtained.

LC-MS (Method 2): Rₜ=0.74 min

MS (ESpos): m/z=454 (M+H)⁺

¹H-NMR (400 MHz, DMSO-d₆): δ=1.08 (s, 3H), 1.71 (br. s, 2H), 1.86-2.00 (m, 2H), 2.36 (s, 3H), 2.56 (s, 3H), 3.22-3.32 (m, 2H; masked by solvent peak), 5.56 (s, 2H), 6.08-6.42 (m, 1H), 7.18-7.26 (m, 2H), 7.53-7.62 (m, 1H), 7.91-8.01 (m, 1H), 8.34 (s, 1H).

### Example 43

### rac-N-(2-Amino-fluoro-2-methylbutyl)-8-[(2,6-difluorobenzyl)oxy]-2,6-dimethylimidazo[1,2-a]pyrazine-3-carboxamide

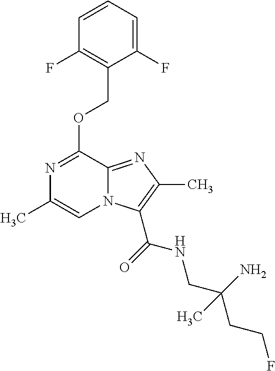 [SMILES: Cc1cn2c(C(=O)NCC(C)(N)CCF)c(C)nc2c(OCc2c(F)cccc2F)n1]

An initial charge of 100 mg (0.30 mmol) of 8-[(2,6-difluorobenzyl)oxy]-2,6-dimethylimidazo[1,2-a]pyrazine-3-carboxylic acid from Example 3A together with 137 mg (0.36 mmol) of HATU and 0.21 ml (1.20 mmol) of N,N-diisopropylethylamine in 1.4 ml of DMF was stirred for 20 min. A solution of 164 mg (0.63 mmol assuming a purity of about 75%) of rac-4-fluoro-2-methylbutane-1,2-diamine dihydrochloride from Example 64A in 0.7 ml of DMF and 0.31 ml (1.80 mmol) of N,N-diisopropylethylamine was added to the first reaction mixture and the mixture was stirred at RT for 0.5 h. The reaction solution was admixed with acetonitrile, water and TFA and purified by means of preparative HPLC (RP18 column, eluent: acetonitrile/water gradient with addition of 0.1% TFA). The product fractions were combined and concentrated. Subsequently, the residue was taken up in dichloromethane and a little methanol, and washed twice with saturated aqueous sodium hydrogencarbonate solution. The aqueous phase was extracted twice with dichloromethane. The combined organic phases were dried over sodium sulphate, filtered, concentrated and lyophilized. 65 mg of the target compound (49% of theory) were obtained.

LC-MS (Method 2): Rₜ=0.73 min

MS (ESpos): m/z=436 (M+H)⁺

¹H-NMR (500 MHz, DMSO-d₆): δ=1.05 (s, 3H), 1.66-2.15 (m, 4H), 2.35 (s, 3H), 2.56 (s, 3H), 3.19-3.32 (m, 2H; partly masked by solvent peak), 4.55-4.63 (m, 1H), 4.66-4.73 (m, 1H), 5.54 (s, 2H), 7.18-7.24 (m, 2H), 7.53-7.61 (m, 1H), 7.91 (br. s, 1H), 8.36 (s, 1H).

### Example 44

### 8-[(2,6-Difluorobenzyl)oxy]-N-(2-hydroxyethoxy)-2,6-dimethylimidazo[1,2-a]pyrazine-3-carboxamide

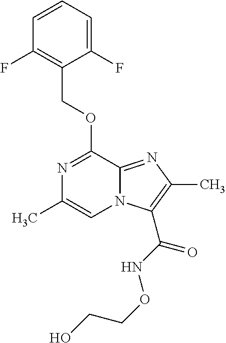 [SMILES: Cc1cn2c(C(=O)NOCCO)c(C)nc2c(OCc2c(F)cccc2F)n1]

33 mg (0.10 mmol) of 8-[(2,6-difluorobenzyl)oxy]-2,6-dimethylimidazo[1,2-a]pyrazine-3-carboxylic acid from Example 3A were initially charged in a 96-well deep well multititre plate. A solution of 8 mg (0.10 mmol) of 2-(aminooxy)ethanol in 0.4 ml of DMF and a solution of 45.6 mg (0.12 mol) of HATU in 0.4 ml of DMF were added successively. After adding 20.2 mg (0.20 mmol) of 4-methylmorpholine, the mixture was shaken at RT overnight. Then the mixture was filtered and the target compound was isolated from the filtrate by preparative LC-MS (Method 9). The product-containing fractions were concentrated under reduced pressure using a centrifugal dryer. The residue of each product fraction was dissolved in 0.6 ml of DMSO. These were combined and finally freed of the solvent in a centrifugal dryer. 0.4 mg (1% of theory) were obtained.

LC-MS (Method 10): Rₜ=0.94 min

MS (ESpos): m/z=393 (M+H)⁺

In analogy to Example 44, the example compounds shown in Table 2 were prepared by reacting the appropriate carboxylic acids with the appropriate amines [hydrazines], which are commercially available or have been described above, under the conditions described:

TABLE 2

| | IUPAC name/structure | |
|---|---|---|
| Example | (Yield) | Analytical data |
| 45 | 8-[(2,6-difluorobenzyl)oxy]-2,6-dimethyl-N- | LC-MS (Method 10): Rₜ = |
| | (morpholin-4-yl)imidazo[1,2-a]pyrazine-3- | 0.97 min |
| | carboxamide | MS (ESpos): m/z = 418 |
| | 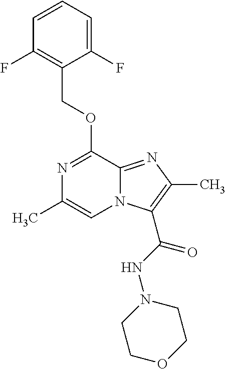 [SMILES: Cc1cn2c(C(=O)NN3CCOCC3)c(C)nc2c(OCc2c(F)cccc2F)n1] | (M + H)⁺ |
| | (13% of theory) | |
| 46 | rac-N-[(4-chlorophenyl)(cyano)methyl]-8-[(2,6- | LC-MS (Method 10): Rₜ = |
| | difluorobenzyl)oxy]-2,6-dimethylimidazo[1,2- | 1.24 min |
| | a]pyrazine-3-carboxamide | MS (ESpos): m/z = 482 |
| | 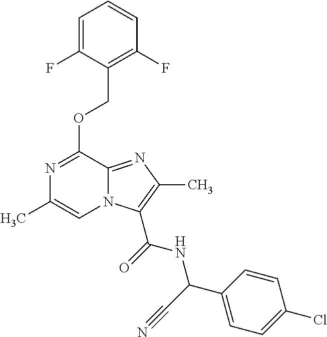 [SMILES: Cc1cn2c(C(=O)NC(C#N)c3ccc(Cl)cc3)c(C)nc2c(OCc2c(F)cccc2F)n1] | (M + H)⁺ |
| | (15% of theory) | |

### Example 47

### 8-(Cyclohexylmethoxy)-N-(3,4-difluorobenzyl)-2-methylimidazo[1,2-a]pyrazine-3-carboxamide

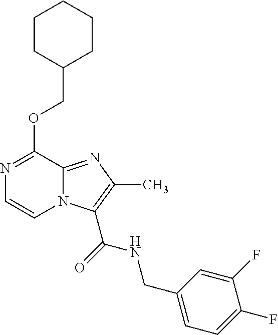 [SMILES: Cc1nc2c(OCC3CCCCC3)nccn2c1C(=O)NCc1ccc(F)c(F)c1]

To a mixture of 3.3 mg (0.08 mmol; 60% purity) of sodium hydride in 0.25 ml of DMF were added 8.5 mg (0.74 mmol) of cyclohexylmethanol and the mixture was stirred at RT for 1 h. Subsequently, a mixture of 25 mg (0.074 mmol) of 8-chloro-N-(3,4-difluorobenzyl)-2-methylimidazo[1,2-a]pyrazine-3-carboxamide from Example 73A in 0.25 ml DMF was added thereto and the reaction mixture was heated to 100° C. After 1.5 h, the mixture was admixed with water and concentrated by evaporation and purified by means of preparative HPLC (RP-C18, eluent: acetonitrile/water gradient with addition of 0.1% formic acid). 7 mg of the title compound were obtained (22% of theory; 95% purity).

LC-MS (Method 2): Rₜ=1.26 min

MS (ESpos): m/z=415 (M+H)⁺

¹H-NMR (400 MHz, DMSO-d₆): δ [ppm]=1.00-1.33 (m, 5H), 1.61-1.90 (m, 6H), 2.61 (s, 3H), 4.27 (d, 2H), 4.52 (d, 2H), 7.20-7.27 (m, 1H), 7.36-7.47 (m, 2H), 7.48 (d, 1H), 8.49 (d, 1H), 8.60 (t, 1H).

### Example 48

### 8-(Cyclobutylmethoxy)-N-(3,4-difluorobenzyl)-2-methylimidazo[1,2-a]pyrazine-3-carboxamide

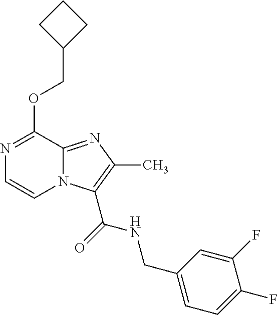 [SMILES: Cc1nc2c(OCC3CCC3)nccn2c1C(=O)NCc1ccc(F)c(F)c1]

To a solution of 27 mg (0.10 mmol) of 8-(cyclobutylmethoxy)-2-methylimidazo[1,2-a]pyrazine-3-carboxylic acid from Example 76A in 1.4 ml of dichloromethane and 0.1 ml of DMF were added 37 mg (0.12 mmol) of (benzotriazol-1-yloxy)bisdimethylaminomethylium fluoroborate (TBTU), 17 mg (0.12 mmol) of 1-(3,4-difluorophenyl)methanamine and 0.057 ml (0.52 mmol) of 4-methylmorpholine, and the mixture was stirred at RT overnight. 2 ml of 10% citric acid were added to the mixture, which was stirred briefly and then filtered through an Extrelut cartridge. The cartridge was washed through well with dichloromethane/ethyl acetate and the filtrate was concentrated by evaporation and the residue was purified by means of preparative HPLC (Macherey-Nagel, VP50/21 Nucleosil 100-5 C18 Nautilus, eluent: acetonitrile/water gradient with addition of 0.1% formic acid). 26 mg of the target compound (66% of theory) were obtained.

LC-MS (Method 2): Rₜ=1.16 min

MS (ESpos): m/z=387 (M+H)⁺

¹H-NMR (400 MHz, DMSO-d₆): δ [ppm]=1.79-1.98 (m, 4H), 2.04-2.16 (m, 2H), 2.60 (s, 3H), 2.75-2.87 (m, 1H), 4.44 (d, 2H), 4.51 (d, 2H), 7.20-7.27 (m, 1H), 7.37-7.47 (m, 2H), 7.49 (d, 1H), 8.49 (d, 1H), 8.60 (t, 1H).

### Example 49

### N-(5-Cyanopentyl)-8-[(2,6-difluorobenzyl)oxy]-2,6-dimethylimidazo[1,2-a]pyrazine-3-carboxamide

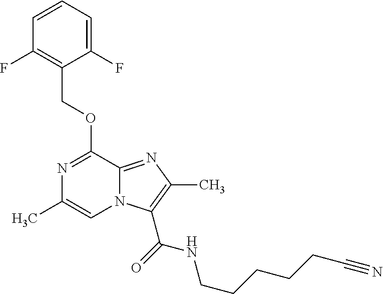 [SMILES: Cc1cn2c(C(=O)NCCCCCC#N)c(C)nc2c(OCc2c(F)cccc2F)n1]

An initial charge of 30 mg (0.09 mmol) of 8-[(2,6-difluorobenzyl)oxy]-2,6-dimethylimidazo[1,2-a]pyrazine-3-carboxylic acid from Example 3A together with 44 mg (0.12 mmol) of HATU and 0.05 ml (0.27 mmol) of N,N-diisopropylethylamine in 0.3 ml of DMF was stirred at room temperature for 20 min. Subsequently, 13 mg (0.12 mmol) of 6-aminohexanenitrile were added to the reaction solution and the mixture was stirred at RT for 30 min. The reaction mixture was admixed with water and stirred at room temperature for 30 min. The solid obtained was filtered off and washed well with water and then dried. 34 mg of the target compound (87% of theory) were obtained.

LC-MS (Method 2): Rₜ=1.03 min

MS (ESpos): m/z=428 (M+H)⁺

¹H-NMR (400 MHz, DMSO-d₆): δ=1.39-1.49 (m, 2H), 1.54-1.68 (m, 4H), 2.36 (s, 3H), 3.27-3.36 (m, 2H; masked by solvent peak), 5.54 (s, 2H), 7.17-7.25 (m, 2H), 7.53-7.63 (m, 1H), 8.09 (t, 1H), 8.36 (s, 1H), [further signals under solvent peak].

In analogy to Example 49, the example compounds shown in Table 3 were prepared by reacting 8-[(2,6-difluorobenzyl)oxy]-2,6-dimethylimidazo[1,2-a]pyrazine-3-carboxylic acid from Example 3A with the appropriate amines [hydrazines, hydrazides, hydroxylamines] which are commercially available or have been described above (1.1-5 equivalents), HATU (1.1-4.5 equivalents) and N,N-diisopropylethylamine (3-12 equivalents) under the reaction conditions described (reaction time: 0.5-48 h; temperature: 0° C.-RT, −20° C., RT or 60° C.).

Illustrative workup of the reaction mixture:

The reaction solution was admixed with water and the precipitated solids were stirred at room temperature for about 30 min. Subsequently, the solids were filtered off, washed well with water and dried under high vacuum.

Alternatively, the reaction mixture was diluted with water/TFA and purified by means of preparative HPLC (RP18 column, eluent: acetonitrile/water gradient with addition of 0.1% TFA or 0.05% formic acid). The crude product was optionally additionally or alternatively purified by means of silica gel chromatography (eluent: dichloromethane/methanol or cyclohexane/ethyl acetate) and/or thick-film chromatography (eluent: dichloromethane/methanol).

The product-containing fractions from the preparative HPLC, if necessary, were concentrated by evaporation and the residue was taken up in dichloromethane and washed with saturated aqueous sodium bicarbonate solution. The aqueous phase was extracted twice with dichloromethane, and the combined organic phases were dried over sodium sulphate, filtered, concentrated and lyophilized.

TABLE 3

| | IUPAC name/structure | |
|---|---|---|
| Example | (Yield) | Analytical data |
| 50 | N-[(4-cyanocyclohexyl)methyl]-8-[(2,6- | LC-MS (Method 2): Rₜ = |
| | difluorobenzyl)oxy]-2,6-dimethylimidazo[1,2- | 1.07 min |
| | a]pyrazine-3-carboxamide trifluoroacetate (cis/trans | MS (ESpos): m/z = 454 (M − |
| | mixture) | TFA + H)⁺ |
| | 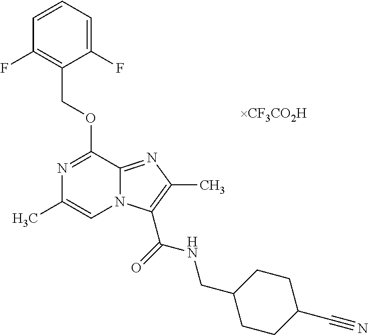 [SMILES: Cc1cn2c(C(=O)NCC3CCC(C#N)CC3)c(C)nc2c(OCc2c(F)cccc2F)n1] | |
| | (67% of theory) | |
| 51 | N-(allyloxy)-8-[(2,6-difluorobenzyl)oxy]-2,6- | LC-MS (Method 2): Rₜ = |
| | dimethylimidazo[1,2-a]pyrazine-3-carboxamide | 0.97 min |
| | trifluoroacetate | MS (ESpos): m/z = 389 |
| | 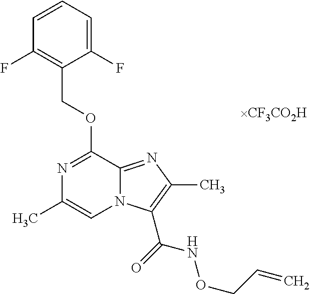 [SMILES: C=CCONC(=O)c1c(C)nc2c(OCc3c(F)cccc3F)nc(C)cn12] | (M + H)⁺ ¹H NMR (500 MHz, DMSO- d₆) δ = 2.38 (s, 3H), 2.48 (s, 3H), 4.47 (d, 2H), 5.31 (d, 1H), 5.40 (d, 1H), 5.55 (s, 2H), 5.97-6.08 (m, 1H), 7.18- 7.25 (m, 2H), 7.53-7.62 (m, 1H), 8.28 (s, 1H). |
| | (50% of theory) | |
| 52 | 8-[(2,6-difluorobenzyl)oxy]-N′-(4-fluorophenyl)-2,6- | LC-MS (Method 2): Rₜ = |
| | dimethylimidazo[1,2-a]pyrazine-3-carbohydrazide | 1.06 min |
| | 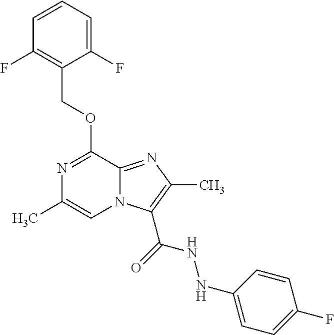 [SMILES: Cc1cn2c(C(=O)NNc3ccc(F)cc3)c(C)nc2c(OCc2c(F)cccc2F)n1] | MS (ESpos): m/z = 442 (M + H)⁺ ¹H NMR (500 MHz, DMSO- d₆) δ = 2.38 (s, 3H), 2.65 (s, 3H), 5.57 (s, 2H), 6.82-6.88 (m, 2H), 6.99-7.08 (m, 2H), 7.18-7.25 (m, 2H), 7.53- 7.62 (m, 1H), 7.00 (br. s, 1H), 8.30 (s, 1H), 9.98 (s, 1H). |
| | (46% of theory) | |
| 53 | 8-[(2,6-difluorobenzyl)oxy]-N′- | LC-MS (Method 2): Rₜ = |
| | [(dimethylamino)acetyl]-2,6-dimethylimidazo[1,2- | 0.61 min |
| | a]pyrazine-3-carbohydrazide | MS (ESpos): m/z = 433 |
| | 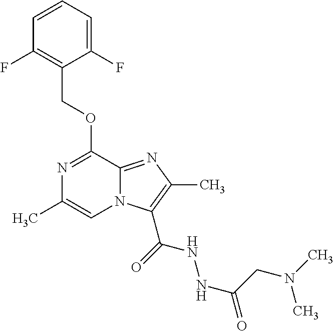 [SMILES: Cc1cn2c(C(=O)NNC(=O)CN(C)C)c(C)nc2c(OCc2c(F)cccc2F)n1] | (M + H)⁺ ¹H NMR (500 MHz, DMSO- d₆) δ = 2.30 (s, 6H), 2.38 (s, 3H), 2.55 (s, 3H), 3.08 (s, 2H), 5.56 (s, 2H), 7.18-7.25 (m, 2H), 7.53-7.62 (m, 1H), 8.28 (s, 1H), 9.87 (br. s, 1H), 10.01 (br. s, 1H). |
| | (66% of theory) | |
| 54 | 8-[(2,6-difluorobenzyl)oxy]-N′-(2-hydroxycyclopentyl)- | LC-MS (Method 2): Rₜ = 0.87 |
| | 2,6-dimethylimidazo[1,2-a]pyrazine-3-carbohydrazide | and 0.89 min |
| | trifluoroacetate (mixture of stereoisomers) | MS (ESpos): m/z = 432 (M − |
| | 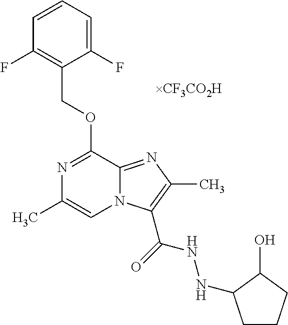 [SMILES: Cc1cn2c(C(=O)NNC3CCCC3O)c(C)nc2c(OCc2c(F)cccc2F)n1] | TFA + H)⁺ |
| | (49% of theory) | |

### B. Assessment of Pharmacological Efficacy

The following abbreviations are used:

ATP adenosine triphosphate
Brij35 polyoxyethylene(23) lauryl ether
BSA bovine serum albumin
DTT dithiothreitol
TEA triethanolamine

The pharmacological action of the compounds of the invention can be demonstrated in the following assays:

### B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant.

Test Procedure

To conduct the test, 29 μl of enzyme solution (0-10 nM soluble guanylyl cyclase (prepared according to Hönicka et al., Journal of Molecular Medicine 77(1999)14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min. Subsequently, 20 μl of detection mix (1.2 nM firefly luciferase (Photinus pyralis Luziferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fraction V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij, pH 7.5) and analysed continuously in a luminometer.

### B-2. Effect on a Recombinant Guanylate Cyclase Reporter Cell Line

The cellular action of the compounds of the invention is determined using a recombinant guanylate cyclase reporter cell line, as described in F. Wunder et al., Anal. Biochem. 339, 104-112 (2005).

Representative MEC values (MEC=minimum effective concentration) for the compounds of the invention are shown in the table below (in some cases as mean values for individual determinations):

TABLE A

| Example | MEC [μM] |
|---|---|
| 1 | 0.77 |
| 2 | 1 |
| 3 | 3 |
| 4 | 1 |
| 5 | 1 |
| 6 | 3 |
| 7 | 0.3 |
| 8 | 1 |
| 9 | 3 |
| 10 | 0.3 |
| 11 | 3 |
| 12 | 1 |
| 13 | 3 |
| 14 | 3 |
| 15 | 1 |
| 16 | 3 |
| 17 | 10 |
| 18 | 3 |
| 19 | 3 |
| 20 | 3 |
| 21 | 3 |
| 22 | 3 |
| 23 | 1 |
| 24 | 3 |
| 25 | 3 |
| 26 | 3 |
| 27 | 1 |
| 28 | 1 |
| 29 | 1 |
| 30 | 3 |
| 31 | 3 |
| 32 | 1 |
| 33 | 3 |
| 34 | 3 |
| 35 | 3 |
| 36 | 1 |
| 37 | 0.3 |
| 38 | 0.3 |
| 39 | 0.2 |
| 40 | 0.3 |
| 41 | 0.65 |
| 42 | 0.3 |
| 43 | 1 |
| 44 | 10 |
| 45 | 10 |
| 46 | 10 |
| 47 | 1.0 |
| 48 | 3.0 |
| 49 | 1.0 |
| 50 | 1.0 |
| 51 | 3.0 |
| 52 | 0.3 |
| 53 | 3.0 |
| 54 | 1.0 |

### B-3. Vasorelaxant Effect In Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each mM): sodium chloride 119; potassium chloride: 4.8; calcium chloride dihydrate: 1; magnesium sulphate heptahydrate: 1.4; potassium dihydrogenphosphate: 1.2; sodium hydrogencarbonate: 25; glucose: 10. The contractile force is determined with Statham UC2 cells, amplified and digitalized using A/D transducers (DAS-1802 HC, Keithley Instruments Munich), and recorded in parallel on linear recorders. To obtain a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

### B-4. Blood Pressure Measurement on Anaesthetized Rats

Male Wistar rats having a body weight of 300-350 g are anaesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

### B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 μm.

### Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

### Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

### Experimental Outline

The telemetry measuring unit present is configured for 24 animals Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number.

In the standard procedure, the following are measured for 10-second periods in each case:

systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

### Evaluation

After the end of the experiment, the acquired individual data are sorted using the analysis software (DATAQUEST™ A.R.T.™ ANALYSIS). The blank value is assumed to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

### LITERATURE

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

### B-6. Determination of Pharmacokinetic Parameters Following Intravenous and Oral Administration

The pharmacokinetic parameters of the compounds of the invention are determined in male CD-1 mice, male Wistar rats and female beagles. Intravenous administration in the case of mice and rats is effected by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is effected at least one day prior to the experiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it can be stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LC-MS/MS using C18 reversed-phase columns and variable eluent mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (by means of LC-MS/MS; see above) and determined by calculating the ratio of the C_{blood}/Cₚₗₐₛₘₐ value.

### B-7. Metabolic Study

To determine the metabolic profile of the compounds of the invention, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with 1:100 dilution into the incubation mixture. Liver microsomes and recombinant enzymes were incubated at 37° C. in 50 mM potassium phosphate buffer pH 7.4 with and without NADPH-generating system consisting of 1 mM NAMP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable eluent mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

### B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammlung von Mikroorganismen und Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analysed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) is also determined in each test run as quality control.

B-9. hERG Potassium Current Assay

The hERG (human ether-a-go-go related gene) potassium current makes a significant contribution to the repolarization of the human cardiac action potential (Scheel et al., 2011). Inhibition of this current by pharmaceuticals can in rare cases cause potentially lethal cardiac arrythmia, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany) NPC-16 chips with moderate resistance (˜2 MΩ; Nanion) serve as the planar substrate for the voltage clamp experiments.

NPC-16 chips are filled with intra- and extracellular solution (cf. Himmel, 2007) and with cell suspension. After forming a gigaohm seal and establishing whole-cell mode (including several automated quality control steps), the cell membrane is clamped at the −80 mV holding potential.

The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 μmol/1) (exposure about 5-6 minutes per concentration), followed by several washing steps.

The amplitude of the upward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A, Neher E, Sakmann B, Sigworth F J Improved patch-clamp techniques for high-resolution current recording from cells and cell-free membrane patches. Pfluegers Arch 1981; 391:85-100.
Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.

### C. Working Examples for Pharmaceutical Compositions

The compounds of the invention can be converted to pharmaceutical formulations as follows:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm

### Production:

The mixture of compound of the invention, lactose and starch is granulated with a 5% solution (w/w) of the PVP in water. The granules are dried and then mixed with the magnesium stearate for 5 minutes. This mixture is compressed in a conventional tabletting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h before swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The resulting solution is subjected to sterile filtration and dispensed into sterile and pyrogen-free injection vessels.

1. Compound of the formula (I) 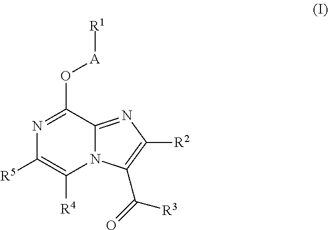
in which
A is CH₂, CD₂ or CH(CH₃),
R¹ is (C₄-C₆)-alkyl, (C₃-C₇)-cycloalkyl, pyridyl or phenyl,
where (C₄-C₆)-alkyl may be up to hexasubstituted by fluorine,
where (C₃-C₇)-cycloalkyl may be substituted by 1 to 4 substituents selected independently from the group of fluorine, trifluoromethyl and (C₁-C₄)-alkyl,
where pyridyl is substituted by 1 or 2 substituents selected independently from the group of halogen, cyano and (C₁-C₄)-alkyl,
and
where phenyl may be substituted by 1 to 4 substituents selected independently from the group of halogen, cyano, monofluoromethyl, difluoromethyl, trifluoromethyl, (C₁-C₄)-alkyl, (C₂-C₃)-alkynyl, (C₁-C₄)-alkoxy, (C₃-C₅)-cycloalkyl, difluoromethoxy and trifluoromethoxy, or may be substituted on two adjacent carbon atoms in the phenyl by a difluoromethylenedioxy bridge,
R² is hydrogen, (C₁-C₄)-alkyl, (C₁-C₄)-alkoxymethyl, cyclopropyl, monofluoromethyl, difluoromethyl or trifluoromethyl,
R³ is a group of the formula 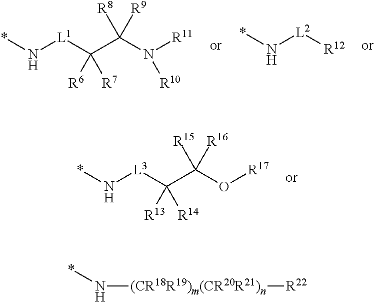
where
* is the attachment site to the carbonyl group,
L¹ is a bond, methanediyl or 1,2-ethanediyl,
in which methanediyl and 1,2-ethanediyl may be substituted by 1 or 2 substituents selected independently from the group of fluorine, trifluoromethyl, (C₁-C₄)-alkyl, (C₃-C₅)-cycloalkyl, hydroxyl and (C₁-C₄)-alkoxy,
L² is a bond or (C₁-C₄)-alkanediyl,
in which (C₁-C₄)-alkanediyl may be substituted by 1 to 3 substituents selected independently from the group of fluorine, trifluoromethyl, (C₁-C₄)-alkyl, (C₃-C₅)-cycloalkyl, hydroxyl and (C₁-C₄)-alkoxy,
L³ is a bond, methanediyl or 1,2-ethanediyl,
in which methanediyl or 1,2-ethanediyl may be substituted by 1 or 2 substituents selected independently from the group of fluorine, trifluoromethyl, (C₁-C₄)-alkyl, (C₃-C₇)-cycloalkyl, hydroxyl and (C₁-C₄)-alkoxy,
R⁶ is hydrogen, (C₁-C₆)-alkyl, (C₂-C₆)-alkenyl, (C₂-C₆)-alkynyl, (C₃-C₇)-cycloalkyl, 5- or 6-membered heteroaryl or phenyl,
in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
in which phenyl, phenoxy and benzyloxy may be substituted by 1 to 3 halogen substituents,
in which (C₃-C₇)-cycloalkyl may be substituted by 1 or 2 substituents selected independently from the group of fluorine, trifluoromethyl, (C₁-C₄)-alkyl and (C₁-C₄)-alkoxy,
and
in which phenyl and 5- or 6-membered heteroaryl may be substituted by 1 to 3 substituents selected independently from the group of halogen, cyano, nitro, trifluoromethyl, (C₁-C₄)-alkyl, (C₁-C₄)-alkoxy, difluoromethoxy, trifluoromethoxy and (C₁-C₄)-alkylsulphonyl,
R⁷ is hydrogen or (C₁-C₆)-alkyl,
or
R⁶ and R⁷ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
R⁸ is hydrogen, (C₁-C₆)-alkyl, (C₃-C₇)-cycloalkyl, (C₂-C₆)-alkenyl, (C₂-C₆)-alkynyl, 5- or 6-membered heteroaryl or phenyl,
in which (C₁-C₆)-alkyl may be substituted by 1 or 2 substituents selected independently from the group of trifluoromethyl, difluoromethoxy, trifluoromethoxy, hydroxyl, (C₁-C₄)-alkoxy, benzyloxy, phenoxy and phenyl, and up to hexasubstituted by fluorine, in which benzyloxy, phenoxy and phenyl may be substituted by 1 to 3 halogen substituents,
in which (C₃-C₇)-cycloalkyl may be substituted by 1 or 2 fluorine or (C₁-C₄)-alkyl substituents,
and
in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R⁹ is hydrogen or (C₁-C₆)-alkyl,
or
R⁸ and R⁹ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
or
R⁶ and R⁸ together with the carbon atoms to which they are bonded form a 3- to 7-membered carbocycle or a 4- to 7-membered heterocycle,
with the proviso that not more than one of the R⁶ and R⁷, R⁸ and R⁹, and R⁶ and R⁸ radical pairs at the same time forms a carbo- or heterocycle,
with the proviso that the R⁶ and R⁸ radicals are not both simultaneously phenyl or 5- or 6-membered heteroaryl,
R¹⁰ is hydrogen or (C₁-C₄)-alkyl,
in which (C₁-C₄)-alkyl may be substituted by 1 to 3 substituents selected independently from the group of fluorine, trifluoromethyl, hydroxyl and (C₁-C₄)-alkoxy,
R¹¹ is hydrogen, (C₁-C₆)-alkyl, (C₃-C₇)-cycloalkyl, phenyl or benzyl,
in which (C₁-C₆)-alkyl may be substituted by 1 to 3 substituents selected independently from the group of fluorine, trifluoromethyl, hydroxyl, (C₁-C₄)-alkoxy and phenoxy,
and
in which phenyl and benzyl may be substituted by 1 to 3 substituents selected independently from the group of halogen and trifluoromethyl,
or
R¹⁰ and R¹¹ together with the nitrogen atom to which they are bonded form a 4- to 7-membered azaheterocycle,
R¹² is 5- to 10-membered azaheterocyclyl bonded via a ring carbon atom,
in which 5- to 10-membered azaheterocyclyl bonded via a ring carbon atom may be substituted by 1 or 2 substituents selected independently from the group of trifluoromethyl, (C₃-C₇)-cycloalkyl, oxo and benzyl, and up to tetrasubstituted by (C₁-C₄)-alkyl and up to disubstituted by fluorine,
and
in which 5- to 10-membered azaheterocyclyl bonded via a ring carbon atom may be fused to a phenyl ring which may in turn be substituted by 1 or 2 substituents selected from halogen, (C₁-C₄)-alkyl and trifluoromethyl,
or
may be amino when L² is a bond,
in which amino may be substituted by (C₁-C₁₀)-alkyl, (C₁-C₄)-alkylcarbonyl, (C₃-C₆)-carbocyclyl, 4- to 7-membered heterocyclyl, phenyl or 5- or 6-membered heteroaryl,
in which (C₁-C₄)-alkylcarbonyl may be substituted by monoalkylamino or dialkylamino,
in which (C₃-C₆)-carbocyclyl and 4- to 7-membered heterocyclyl may be substituted by hydroxyl,
and
in which phenyl and 5- or 6-membered heteroaryl may be substituted by 1 to 3 substituents selected independently from the group of halogen, (C₁-C₄)-alkyl and trifluoromethyl,
R¹³ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, —(C═O)NR²³R²⁴, 5- or 6-membered heteroaryl or phenyl,
in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
in which phenyl, phenoxy and benzyloxy may in turn be substituted by 1 to 3 halogen substituents,
in which (C₃-C₇)-cycloalkyl may be substituted by 1 or 2 substituents selected independently from the group of fluorine, trifluoromethyl, (C₁-C₄)-alkyl and (C₁-C₄)-alkoxy,
in which R²³ is hydrogen, (C₁-C₄)-alkyl, (C₃-C₇)-cycloalkyl, aryl or naphthyl,
in which R²⁴ is hydrogen or methyl,
and
in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁴ is hydrogen or (C₁-C₆)-alkyl,
in which (C₁-C₄)-alkyl may be substituted by hydroxyl,
or
R¹³ and R¹⁴ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
R¹⁵ is hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, 5- or 6-membered heteroaryl or phenyl,
in which (C₁-C₆)-alkyl may be substituted by 1 or 2 substituents selected independently from the group of difluoromethoxy, trifluoromethoxy, hydroxyl, (C₁-C₄)-alkoxy, (C₁-C₄)-alkoxycarbonyl, (C₁-C₄)-alkylthio, (C₁-C₄)-alkylsulphonyl, phenyl, phenoxy and benzyloxy, and up to hexasubstituted by fluorine,
in which phenyl, phenoxy and benzyloxy may in turn be substituted by 1 to 3 halogen substituents,
in which (C₃-C₇)-cycloalkyl may be substituted by 1 or 2 substituents selected independently from the group of fluorine, trifluoromethyl, (C₁-C₄)-alkyl and (C₁-C₄)-alkoxy,
and
in which phenyl and 5- or 6-membered heteroaryl may be substituted by 1 to 3 substituents selected independently from the group of halogen, cyano, trifluoromethyl, (C₁-C₄)-alkyl, (C₁-C₄)-alkoxy and (C₁-C₄)-alkylsulphonyl,
R¹⁶ is hydrogen or (C₁-C₆)-alkyl,
in which (C₁-C₄)-alkyl may be substituted by hydroxyl,
or
R¹⁵ and R¹⁶ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
with the proviso that the R¹³ and R¹⁵ radicals are not both simultaneously phenyl or 5- or 6-membered heteroaryl,
or
R¹³ and R¹⁵ together with the carbon atoms to which they are bonded form a 3- to 7-membered carbocycle or a 4- to 7-membered heterocycle,
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
in which (C₁-C₄)-alkyl may be substituted by 1 to 5 fluorine substituents,
m is 0, 1 or 2,
n is 0 or 1,
R¹⁸ is hydrogen, cyano or (C₁-C₄)-alkyl,
in which (C₁-C₄)-alkyl may be substituted by 1 to 5 fluorine substituents,
R¹⁹ is hydrogen or (C₁-C₄)-alkyl,
in which (C₁-C₄)-alkyl may be substituted by 1 to 5 fluorine substituents,
R²⁰ is hydrogen or (C₁-C₄)-alkyl,
in which (C₁-C₄)-alkyl may be substituted by 1 to 5 fluorine substituents,
R²¹ is hydrogen or (C₁-C₄)-alkyl,
in which (C₁-C₄)-alkyl may be substituted by 1 to 3 fluorine substituents,
or
R¹⁸ and R¹⁹ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
or
R²⁰ and R²¹ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
or
R¹⁸ and R²⁰ together with the carbon atom to which they are bonded form a 3- to 7-membered carbocycle or a 4- to 7-membered heterocycle,
in which the 3- to 7-membered carbocycle and the 4- to 7-membered heterocycle may in turn be substituted by 1 or 2 substituents selected independently from the group of fluorine and (C₁-C₄)-alkyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, cyano, (C₁-C₆)-alkoxy, 5- to 9-membered heterocyclyl bonded via a ring carbon atom, 5- to 9-membered carbocyclyl, phenyl, indanyl or 5- to 10-membered heteroaryl,
in which (C₁-C₆)-alkyl may be substituted by cyano and up to hexasubstituted by fluorine,
in which (C₁-C₆)-alkoxy may be substituted by hydroxyl, amino,
monoalkylamino, dialkylamino, cyclopropyl, phenyl or (C₂-C₄)-alkenyl, in which phenyl may be substituted by 1 to 3 substituents independently of one another selected from the group consisting of halogen, cyano, trifluoromethyl, difluoromethyl, (C₁-C₆)-alkyl, (C₁-C₄)-alkylcarbonyl, (C₁-C₄)-alkoxycarbonyl, hydroxycarbonyl, —(C═O)NR²⁵R²⁶, (C₁-C₄)-alkylsulphonyl, (C₃-C₆)-cycloalkylsulphonyl, (C₁-C₄)-alkylthio, (C₁-C₄)-alkoxy, trifluoromethoxy, difluoromethoxy, phenoxy, hydroxyl, 5- to 10-membered heteroaryl and (C₃-C₇)-cycloalkyl,
in which (C₁-C₆)-alkyl may be substituted by 1 or 2 substituents selected independently from the group consisting of fluorine, trifluoromethoxy, (C₁-C₄)-alkylcarbonyl, —(C═O)NR²⁵R²⁶, C₄)-alkoxy, (C₃-C₆)-cycloalkyl, morpholinyl, piperidinyl, pyrrolidinyl, piperazinyl, phenyl, hydroxyl and amino,
in which phenyl may be substituted by 1 to 3 halogen substituents,
in which amino may be substituted by 1 or 2 substituents selected independently from (C₁-C₆)-alkyl, (C₁-C₄)-alkylcarbonyl, (C₃-C₆)-cycloalkylsulphonyl, (C₁-C₄)-alkylsulphonyl and methoxy-(C₁-C₄)-alkyl,
in which (C₃-C₆)-cycloalkyl may be substituted by amino or hydroxyl, and in which
R²⁵ and R²⁶ are each independently hydrogen, (C₁-C₄)-alkyl or (C₃-C₇)-cycloalkyl,
in which indanyl may be substituted by 1 or 2 substituents selected independently from the group of fluorine, trifluoromethyl and hydroxyl,
in which 5- to 10-membered heteroaryl may be substituted by 1 to 3 substituents independently of one another selected from the group of fluorine, chlorine, cyano, (C₁-C₆)-alkyl, trifluoromethyl, (C₁-C₄)-alkoxy, amino, (C₁-C₄)-alkoxycarbonyl, hydroxycarbonyl, —(C═O)NR²⁵R²⁶, phenyl, pyridyl, pyrimidyl, 1,3-thiazol-5-yl and (C₃-C₇)-cycloalkyl,
in which (C₁-C₆)-alkyl may be substituted by 1 to 3 substituents selected independently from the group of halogen, cyano, hydroxyl, amino, trifluoromethyl, difluoromethyl, (C₁-C₄)-alkylsulphonyl, (C₁-C₄)-alkylcarbonyl, (C₁-C₄)-alkoxycarbonyl, hydroxycarbonyl, (C₁-C₄)-alkylthio, (C₁-C₄)-alkoxy, trifluoromethoxy, difluoromethoxy, phenoxy, phenyl, pyridyl, pyrimidyl, 5-membered heteroaryl, tetrahydrothiophenyl 1,1-dioxide, (C₃-C₇)-cycloalkyl, morpholinyl, piperidinyl, pyrrolidinyl, 2-oxopyrrolidin-1-yl, piperazinyl, tetrahydrothiophenyl 1,1-dioxide, thiomorpholinyl 1,1-dioxide and azetidine,
in which 5-membered heteroaryl may be substituted by 1 to 3 substituents selected independently from the group of halogen, (C₁-C₄)-alkyl and (C₁-C₄)-alkoxy,
in which piperidinyl may be substituted by 1 to 4 fluorine substituents,
in which phenyl may be substituted by 1 to 3 substituents selected independently from the group of halogen, (C₁-C₄)-alkyl and (C₁-C₄)-alkoxy,
in which azetidine may be substituted by hydroxyl,
in which piperazinyl may be substituted by 1 to 3 substituents selected independently from the group consisting of (C₁-C₄)-alkyl, (C₃-C₇)-cycloalkyl and trifluoromethyl,
and in which
R²⁵ and R²⁶ are each independently hydrogen, (C₁-C₄)-alkyl or (C₃-C₇)-cycloalkyl,
in which 5- to 9-membered heterocyclyl bonded via a ring carbon atom may be substituted by 1 or 2 substituents selected independently from the group of oxo, fluorine, hydroxyl and (C₁-C₄)-alkyl,
and
in which 5- to 9-membered carbocyclyl may be substituted by 1 or 2 substituents selected independently from the group of trifluoromethyl, fluorine, cyano, hydroxyl, hydroxycarbonyl, (C₁-C₄)-alkoxycarbonyl and (C₁-C₄)-alkyl,
R⁴ is hydrogen,
R⁵ is hydrogen, halogen, cyano, difluoromethyl, trifluoromethyl, (C₁-C₄)-alkyl, (C₃-C₇)-cycloalkyl, (C₂-C₄)-alkynyl, (C₁-C₄)-alkylamino, difluoromethoxy, trifluoromethoxy, (C₁-C₄)-alkoxy, amino, 4- to 7-membered heterocyclyl or 5- or 6-membered heteroaryl,
and the N-oxides, salts, solvates, salts of the N-oxides and solvates of the N-oxides or salts thereof.

2. The compound of claim 1, in which
A is CH₂ or CD₂,
R¹ is (C₃-C₆)-cycloalkyl, pyridyl or phenyl,
where (C₃-C₆)-cycloalkyl may be substituted by 1 to 2 substituents selected independently from the group of fluorine, trifluoromethyl, methyl and ethyl,
where pyridyl is substituted by 1 or 2 fluorine substituents,
and
where phenyl may be substituted by 1 to 4 substituents selected independently from the group of halogen, cyano, difluoromethyl, trifluoromethyl, (C₁-C₄)-alkyl, (C₁-C₄)-alkoxy and (C₃-C₅)-cyclopropyl,
R² is hydrogen, (C₁-C₄)-alkyl, cyclopropyl, difluoromethyl or trifluoromethyl,
R³ is a group of the formula 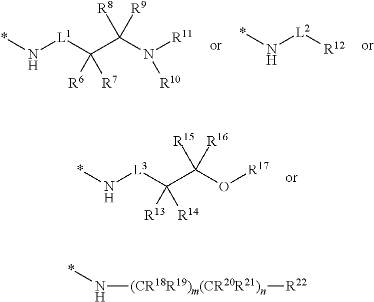
where
* is the attachment site to the carbonyl group,
L¹ is a bond, methanediyl or 1,2-ethanediyl,
L² is a bond, methanediyl or 1,2-ethanediyl,
L³ is a bond, methanediyl or 1,2-ethanediyl,
R⁶ is hydrogen, (C₁-C₆)-alkyl, (C₃-C₇)-cycloalkyl, phenyl or 5- or 6-membered heteroaryl,
in which (C₁-C₆)-alkyl may be substituted up to five times by fluorine,
and
in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁷ is hydrogen or (C₁-C₄)-alkyl,
or
R⁶ and R⁷ together with the carbon atom to which they are bonded form a 3- to 5-membered carbocycle,
R⁸ is hydrogen, (C₁-C₆)-alkyl, (C₃-C₅)-cycloalkyl, 5- or 6-membered heteroaryl or phenyl,
in which (C₁-C₆)-alkyl may be substituted by (C₁-C₄)-alkoxy, benzyloxy or phenoxy, and up to pentasubstituted by fluorine,
in which benzyloxy and phenoxy may be substituted by 1 to 3 substituents selected independently from the group of fluorine, chlorine and bromine,
in which (C₃-C₅)-cycloalkyl may be substituted by 1 or 2 fluorine or (C₁-C₄)-alkyl substituents,
and
in which phenyl and 5- or 6-membered heteroaryl may be substituted by 1 to 3 substituents selected independently from the group of fluorine, chlorine, bromine, cyano, trifluoromethyl, methyl, ethyl, methoxy or ethoxy,
R⁹ is hydrogen or (C₁-C₄)-alkyl,
or
R⁸ and R⁹ together with the carbon atom to which they are bonded form a 3- to 5-membered carbocycle,
or
R⁶ and R⁸ together with the carbon atom to which they are bonded form a 3- to 6-membered carbocycle or a 4- to 7-membered heterocycle,
in which the 3- to 6-membered carbocycle and the 4- to 7-membered heterocycle may be substituted by 1 or 2 fluorine or (C₁-C₄)-alkyl substituents,
with the proviso that not more than one of the R⁶ and R⁷, R⁸ and R⁹, and R⁶ and R⁸ radical pairs at the same time forms a carbo- or heterocycle,
and
with the proviso that the R⁶ and R⁸ radicals are not both simultaneously phenyl or 5- or 6-membered heteroaryl,
R¹⁰ is hydrogen or (C₁-C₄)-alkyl,
in which (C₁-C₄)-alkyl may be substituted up to five times by fluorine,
R¹¹ is hydrogen, (C₁-C₆)-alkyl or (C₃-C₇)-cycloalkyl,
in which (C₁-C₆)-alkyl may be substituted up to five times by fluorine,
or
R¹⁰ and R¹¹ together with the nitrogen atom to which they are bonded form a 4- to 7-membered azaheterocycle,
R¹² is 5- to 10-membered azaheterocyclyl bonded via a ring carbon atom,
in which 5- to 10-membered azaheterocyclyl may be substituted by 1 to 5 substituents selected independently from the group of fluorine, methyl and ethyl,
or
may be amino when L² is a bond,
in which amino may be substituted by (C₁-C₄)-alkyl, (C₁-C₄)-alkylcarbonyl, (C₃-C₆)-carbocyclyl, 4- to 7-membered heterocyclyl, phenyl or 5- or 6-membered heteroaryl,
in which (C₁-C₄)-alkylcarbonyl may be substituted by monoalkylamino or dialkylamino,
in which (C₃-C₆)-carbocyclyl and 4- to 7-membered heterocyclyl may be substituted by hydroxyl,
and
in which phenyl and 5- or 6-membered heteroaryl may be substituted by 1 to 3 substituents selected independently from the group of fluorine, chlorine, methyl and trifluoromethyl,
R¹³ is hydrogen, (C₁-C₆)-alkyl, (C₃-C₅)-cycloalkyl, —(C═O)NR²³R²⁴, 5- or 6-membered heteroaryl or phenyl,
in which (C₁-C₆)-alkyl may be substituted by 1 or 2 substituents selected independently from the group of difluoromethoxy, trifluoromethoxy, hydroxyl and (C₁-C₄)-alkoxy, and up to hexasubstituted by fluorine,
in which R²³ is hydrogen, (C₁-C₄)-alkyl, aryl or naphthyl,
in which R²⁴ is hydrogen,
and
in which phenyl and 5- or 6-membered heteroaryl may be substituted by 1 to 3 substituents selected independently from the group of fluorine, chlorine, bromine, trifluoromethyl, methyl and ethyl,
R¹⁴ is hydrogen or (C₁-C₄)-alkyl,
or
R¹³ and R¹⁴ together with the carbon atom to which they are bonded form a 3- to 5-membered carbocycle,
R¹⁵ is hydrogen, (C₁-C₆)-alkyl or (C₃-C₅)-cycloalkyl,
in which (C₁-C₆)-alkyl may be substituted up to five times by fluorine,
and
in which (C₃-C₅)-cycloalkyl may be substituted by 1 or 2 substituents selected independently from the group consisting of fluorine, trifluoromethyl, hydroxyl and (C₁-C₄)-alkyl,
R¹⁶ is hydrogen or (C₁-C₄)-alkyl,
or
R¹⁵ and R¹⁶ together with the carbon atom to which they are bonded form a 3- to 6-membered carbocycle,
in which the 3- to 6-membered carbocycle may be substituted by 1 or 2 fluorine or (C₁-C₄)-alkyl substituents,
or
R¹³ and R¹⁵ together with the carbon atom to which they are bonded form a 3- to 6-membered carbocycle or a 4- to 7-membered heterocycle,
in which the 3- to 6-membered carbocycle and the 4- to 7-membered heterocycle may be substituted by 1 or 2 fluorine or (C₁-C₄)-alkyl substituents,
with the proviso that the R¹³ and R¹⁵ radicals are not both simultaneously phenyl,
and
with the proviso that not more than one of the R¹³ and R¹⁴, R¹⁵ and R¹⁶, and R¹³ and R¹⁵ radical pairs at the same time forms a carbo- or heterocycle,
R¹⁷ is hydrogen or (C₁-C₄)-alkyl,
in which (C₁-C₄)-alkyl may be substituted by 1 to 5 fluorine substituents,
m is 0 or 1,
n is 0 or 1,
R¹⁸ is hydrogen, cyano or (C₁-C₄)-alkyl,
in which (C₁-C₄)-alkyl may be substituted by 1 to 5 fluorine substituents,
R¹⁹ is hydrogen or (C₁-C₄)-alkyl,
in which (C₁-C₄)-alkyl may be substituted by 1 to 5 fluorine substituents,
R²⁰ is hydrogen or (C₁-C₄)-alkyl,
in which (C₁-C₄)-alkyl may be substituted by 1 to 5 fluorine substituents,
R²¹ is hydrogen or (C₁-C₄)-alkyl,
in which (C₁-C₄)-alkyl may be substituted by 1 to 5 fluorine substituents,
or
R¹⁸ and R¹⁹ together with the carbon atom to which they are bonded form a 3- to 5-membered carbocycle,
in which the 3- to 5-membered carbocycle may be substituted by 1 or 2 substituents selected independently from the group of fluorine, methyl and ethyl,
or
R²⁰ and R²¹ together with the carbon atom to which they are bonded form a 3- to 5-membered carbocycle,
in which the 3- to 5-membered carbocycle may be substituted by 1 or 2 substituents selected independently from the group of fluorine, methyl and ethyl,
or
R¹⁸ and R²⁰ together with the carbon atom to which they are bonded form a 3- to 5-membered carbocycle,
in which the 3- to 5-membered carbocycle may be substituted by 1 or 2 substituents selected independently from the group of fluorine, methyl and ethyl,
with the proviso that not more than one of the R¹⁸ and R¹⁹, R²⁰ and R²¹, and R¹⁸ and R²⁰ radical pairs at the same time forms a carbo- or heterocycle,
R²² is (C₁-C₆)-alkyl, cyano, (C₁-C₆)-alkoxy, 5- to 9-membered heterocyclyl bonded via a ring carbon atom, 5- to 9-membered carbocyclyl, phenyl, indanyl or 5- to 10-membered heteroaryl,
in which (C₁-C₆)-alkyl may be substituted by cyano or up to pentasubstituted by fluorine,
in which (C₁-C₆)-alkoxy may be substituted by hydroxyl or (C₂-C₄)-alkenyl,
in which phenyl may be substituted by 1 to 3 substituents selected independently from the group of halogen, cyano, trifluoromethyl, difluoromethyl, (C₁-C₄)-alkyl, (C₁-C₄)-alkoxy and 5- to 10-membered heteroaryl,
in which (C₁-C₄)-alkyl may be substituted by 1 or 2 substituents selected independently from the group of fluorine, trifluoromethoxy, (C₁-C₄)-alkoxy, (C₃-C₆)-cycloalkyl, hydroxyl and amino,
in which indanyl may be substituted by 1 or 2 substituents selected independently from the group of fluorine, trifluoromethyl and hydroxyl,
in which 5- to 10-membered heteroaryl may be substituted by 1 to 3 substituents selected independently from the group of fluorine, chlorine, cyano, trifluoromethyl, (C₁-C₄)-alkyl, (C₁-C₄)-alkoxy, amino and hydroxyl,
in which (C₁-C₄)-alkyl may be substituted by 1 to 3 substituents selected independently from the group of halogen, cyano, hydroxyl, amino, trifluoromethyl, difluoromethyl, (C₁-C₄)-alkoxy, trifluoromethoxy, difluoromethoxy and phenyl,
in which phenyl may be substituted by 1 to 3 halogen substituents,
in which 5- to 9-membered heterocyclyl bonded via a ring carbon atom may be substituted by 1 or 2 substituents selected independently from the group of oxo, fluorine, hydroxyl and (C₁-C₄)-alkyl,
and
in which 5- to 9-membered carbocyclyl may be substituted by 1 or 2 substituents selected independently from the group of trifluoromethyl, fluorine, hydroxyl, hydroxycarbonyl, (C₁-C₄)-alkoxycarbonyl and (C₁-C₄)-alkyl,
R⁴ is hydrogen,
R⁵ is hydrogen, fluorine, chlorine, bromine, cyano, difluoromethyl, trifluoromethyl, (C₁-C₄)-alkyl, (C₂-C₄)-alkynyl or (C₃-C₅)-cycloalkyl,
and the N-oxides, salts, solvates, salts of the N-oxides and solvates of the N-oxides or salts thereof.

3. The compound of claim 1 in which
A is CH₂,
R¹ is cyclohexyl, pyridyl or phenyl,
where cyclohexyl may be substituted by 1 to 2 substituents selected independently from the group of fluorine and methyl,
where pyridyl is substituted by 1 or 2 F substituents,
and
where phenyl may be substituted by 1 to 4 substituents selected independently from the group of fluorine, chlorine, methyl, methoxy and cyclopropyl,
R² is methyl, cyclopropyl or trifluoromethyl,
R³ is a group of the formula 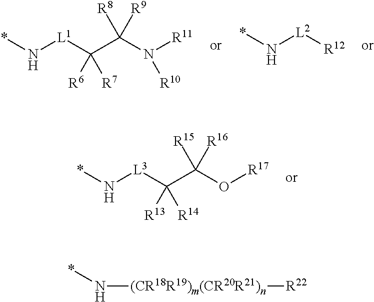
where
* is the attachment site to the carbonyl group,
L¹ is a bond,
L² is a bond,
L³ is a bond,
R⁶ is hydrogen, (C₁-C₆)-alkyl or phenyl,
in which (C₁-C₆)-alkyl may be substituted up to five times by fluorine,
and
in which phenyl may be substituted by 1 to 3 substituents selected independently from the group of fluorine, chlorine, trifluoromethyl, methyl and methoxy,
R⁷ is hydrogen, methyl or ethyl,
R⁸ is hydrogen, (C₁-C₆)-alkyl, cyclopropyl or cyclobutyl,
in which (C₁-C₆)-alkyl may be substituted up to five times by fluorine,
R⁹ is hydrogen or (C₁-C₄)-alkyl,
Or
R⁸ and R⁹ together with the carbon atom to which they are bonded form a 3- to 6-membered carbocycle,
R¹⁰ is hydrogen, methyl or ethyl,
in which ethyl may be substituted up to three times by fluorine,
R¹¹ is hydrogen, (C₁-C₄)-alkyl or (C₃-C₅)-cycloalkyl,
Or
R¹⁰ and R¹¹ together with the nitrogen atom to which they are bonded form a morpholinyl ring or piperidinyl ring,
R¹² is 9-azabicyclo[3.3.1]nonan-3-yl or piperidin-4-yl,
in which 9-azabicyclo[3.3.1]nonan-3-yl is substituted by methyl,
in which piperidin-4-yl is substituted by 1 to 5 methyl substituents,
R¹³ is hydrogen, (C₁-C₆)-alkyl, —(C═O)NR²³R²⁴ or phenyl,
in which (C₁-C₆)-alkyl may be substituted by a hydroxyl or methoxy radical or up to five times by fluorine,
in which R²³ is aryl or naphthyl,
in which R²⁴ is hydrogen,
and
in which phenyl may be substituted by 1 to 3 substituents selected independently from the group consisting of fluorine, chlorine, trifluoromethyl and methyl,
R¹⁴ is hydrogen or (C₁-C₄)-alkyl,
R¹⁵ is hydrogen, (C₁-C₆)-alkyl, cyclopropyl or cyclobutyl,
in which (C₁-C₆)-alkyl may be substituted up to five times by fluorine,
in which cyclopropyl and cyclobutyl may be substituted by 1 or 2 substituents selected independently from the group of fluorine and methyl,
R¹⁶ is hydrogen or (C₁-C₄)-alkyl,
Or
R¹⁵ and R¹⁶ together with the carbon atom to which they are bonded form a 3- to 6-membered carbocycle,
in which the 3- to 6-membered carbocycle may be substituted by 1 or 2 fluorine or methyl substituents,
R¹⁷ is hydrogen or (C₁-C₄)-alkyl,
in which (C₁-C₃)-alkyl may be substituted by 1 to 5 fluorine substituents,
m is 0 or 1,
n is 0 or 1,
R¹⁸ is hydrogen, cyano or methyl,
in which methyl may be substituted by 1 to 3 fluorine substituents,
R¹⁹ is hydrogen or methyl,
in which methyl may be substituted by 1 to 3 fluorine substituents,
R²⁰ is hydrogen or methyl,
in which methyl may be substituted by 1 to 3 fluorine substituents,
R²¹ is hydrogen or methyl,
in which methyl may be substituted by 1 to 3 fluorine substituents,
or
R¹⁸ and R¹⁹ together with the carbon atom to which they are bonded form a 3- to 5-membered carbocycle,
Or
R¹⁸ and R²⁰ together with the carbon atom to which they are bonded form a cyclopropyl ring,
with the proviso that not more than one of the R¹⁸ and R¹⁹, and R¹⁸ and R²⁰ radical pairs at the same time forms a carbocycle,
R²² is (C₁-C₆)-alkyl, cyano, 2-oxopyrrolidin-3-yl, 2-oxotetrahydrofuran-3-yl, cyclopentyl, cyclohexyl, phenyl, indanyl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
in which (C₁-C₆)-alkyl may be substituted by a cyano radical or up to trisubstituted by fluorine,
where phenyl may be substituted by 1 to 3 substituents selected independently from the group of fluorine, chlorine, cyano, trifluoromethyl, methyl, ethyl, methoxy and pyridyl,
where indanyl may be substituted by hydroxyl,
where 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl may be substituted by 1 to 3 substituents selected independently from the group of fluorine, chlorine, trifluoromethyl, (C₁-C₃)-alkyl, amino and hydroxyl,
in which (C₁-C₃)-alkyl may be substituted by fluorine, hydroxyl, amino or trifluoromethyl,
where cyclopentyl and cyclohexyl are substituted by cyano, methoxycarbonyl or ethoxycarbonyl,
R⁴ is hydrogen,
R⁵ is hydrogen, methyl or ethyl,
and the N-oxides, salts, solvates, salts of the N-oxides and solvates of the N-oxides or salts thereof.

4. The compound of claim 1, in which
A is CH₂,
R¹ is a phenyl group of the formula 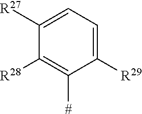
where
# is the attachment site to A,
And
R²⁷ is hydrogen or fluorine,
R²⁸ is fluorine,
R²⁹ is fluorine,
R² is methyl,
R³ is a group of the formula 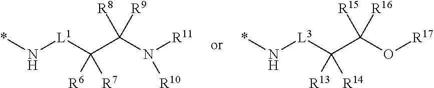
where
* is the attachment site to the carbonyl group,
L¹ is a bond,
L³ is a bond,
R⁶ is hydrogen, (C₁-C₆)-alkyl or phenyl,
in which (C₁-C₆)-alkyl may be substituted up to five times by fluorine,
and
in which phenyl may be substituted by 1 to 2 chlorine or fluorine substituents,
R⁷ is hydrogen, methyl or ethyl,
R⁸ is hydrogen, (C₁-C₆)-alkyl, trifluoromethyl or cyclopropyl,
in which (C₁-C₆)-alkyl may be up to trisubstituted by fluorine,
R⁹ is hydrogen, methyl or ethyl,
R¹⁰ is hydrogen,
R¹¹ is hydrogen,
R¹³ is hydrogen, (C₁-C₆)-alkyl or phenyl,
in which (C₁-C₆)-alkyl may be substituted by a hydroxyl radical or up to five times by fluorine,
and
in which phenyl may be substituted by 1 or 2 fluorine substituents,
R¹⁴ is hydrogen, methyl or ethyl,
R¹⁵ is hydrogen or (C₁-C₆)-alkyl,
R¹⁶ is hydrogen, methyl or ethyl,
or
R¹⁵ and R¹⁶ together with the carbon atom to which they are bonded form a 3- to 6-membered carbocycle,
R¹⁷ is hydrogen,
R⁴ is hydrogen,
R⁵ is hydrogen or methyl,
and the N-oxides, salts, solvates, salts of the N-oxides and solvates of the N-oxides or salts thereof.

5. A process for preparing the compound of claim 1, comprising
[A] converting a compound of the formula (II) 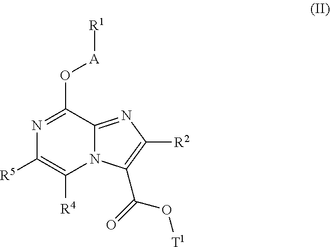
in which A, R¹, R², R⁴ and R⁵ are each as defined in claim 1 and
T¹ is (C₁-C₄)-alkyl or benzyl,
in an inert solvent in the presence of a suitable base or acid into a carboxylic acid of the formula (III) 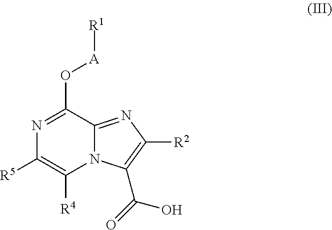
in which A, R¹, R², R⁴ and R⁵ are each as defined in claim 1,
and subsequently reacting the carboxylic acid of the formula (III), in an inert solvent under amide coupling conditions, with an amine of the formula (IV-A), (IV-B), (IV-C) or (IV-D) 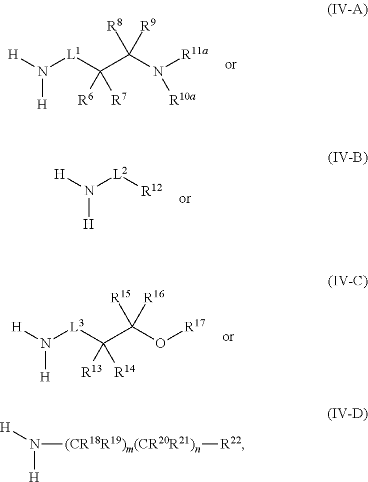
in which L¹, L², L³, R⁶, R⁷, R⁸, R⁹, R¹², R¹³, R¹⁴, R¹⁵, R¹⁶, R¹⁷, R¹⁸, R¹⁹, R²⁰, R²¹ and R²² are each as defined in claim 1
and
R^{10A} and R^{11A} are each as defined above for R¹⁰ and R¹¹ or are an amino protecting group,
then detaching any protecting groups present, and optionally converting the resulting compounds of the formula (I) with the appropriate (i) solvents and/or (ii) acids or bases to the solvates, salts and/or solvates of the salts thereof.

6. (canceled)

7. (canceled)

8. A medicament comprising the compound of claim 1 in combination with an inert, non-toxic, pharmaceutically suitable excipient.

9. A medicament comprising the compound of claim 1 in combination with a further active ingredient selected from the group consisting of organic nitrates, NO donors, cGMP-PDE inhibitors, antithrombotic agents, hypotensive agents and lipid metabolism modifiers.

10. A method for the treatment and/or prophylaxis of heart failure, angina pectoris, hypertension, pulmonary hypertension, ischaemias, vascular disorders, kidney failure, thromboembolic disorders and arteriosclerosis in humans and animals, the method comprising administering an effective amount of the medicament of claim 8 to a human or animal in need thereof.

11. A method for treatment and/or prophylaxis of heart failure, angina pectoris, hypertension, pulmonary hypertension, ischaemias, vascular disorders, renal insufficiency, thromboembolic disorders and arteriosclerosis in humans and animals, the method comprising administering acing an effective amount of at least one compound of the claim 1 to a human or animal in need thereof.
